(19) United States
(12) Patent Application Publication
(10) Pub. No.: US 2025/0333509 A1
(43) Pub. Date: Oct. 30, 2025

(54) METHODS FOR TREATING CANCER, OR VON-HIPPEL LINDAU DISEASE USING A COMBINATION OF A TIGIT ANTAGONIST, A PD-1 ANTAGONIST, AND A HIF-2-ALPHA INHIBITOR

(71) Applicant: MERCK SHARP & DOHME LLC, Rahway, NJ (US)

(72) Inventors: Joseph E. BURGENTS, Line Lexington, PA (US); Rodolfo Fleury PERINI, Wynnewood, PA (US); Tanya KEENAN, Brookline, MA (US)

(73) Assignee: MERCK SHARP & DOHME LLC, Rahway, NJ (US)

(21) Appl. No.: 18/869,398

(22) PCT Filed: May 26, 2023
(86) PCT No.: PCT/US2023/023635

§ 371 Date: Nov. 26, 2024

Related U.S. Application Data
(60) Provisional application No. 63/348,203, filed on Jun. 2, 2022.

(51) Int. Cl. C07K 16/28; A61K 31/277; A61K 39/00; A61P 35/00

(52) U.S. Cl. CPC C07K 16/2818; A61K 31/277; A61P 35/00; A61K 2039/507; A61K 2039/545

(57) ABSTRACT

Provided herein are methods of treating cancer, or von-Hippel Lindau disease, which comprise administering to a human patient in need thereof: (a) a TIGIT antagonist; (b) a PD-1 antagonist; and an HIF-2α inhibitor. Also provided are kits containing such agents and uses of therapeutic combinations of such agents for the treatment of cancer, or von-Hippel Lindau disease.

### FIELD

Provided herein are methods for treating cancer, or von-Hippel Lindau disease using a combination of (a) a T cell immunoreceptor with Ig and ITIM domains (TIGIT) antagonist, (b) a programmed death 1 protein (PD-1) antagonist, and (c) a Hypoxia-Inducible Factors-2alpha (HIF-2α) inhibitor.

### REFERENCE TO SEQUENCE LISTING SUBMITTED ELECTRONICALLY

The instant application contains a Sequence Listing which has been submitted electronically in XML format and is hereby incorporated by reference in its entirety. The XML file, created on Apr. 26, 2023, is named 25394-US-PCT_SL.XML and is 355,055 bytes in size.

### BACKGROUND OF THE INVENTION

TIGIT is an immunomodulatory receptor expressed primarily on activated T cells and NK cells. TIGIT is also known as VSIG9, VSTM3, and WUCAM. Its structure shows one extracellular immunoglobulin domain, a type 1 transmembrane region and two ITIM motifs. TIGIT forms part of a co-stimulatory network that consists of positive (CD226) and negative (TIGIT) immunomodulatory receptors on T cells, and ligands expressed on antigen presenting cells (APCs) (CD155 and CD112).

An important feature in the structure of TIGIT is the presence of an immunoreceptor tyrosine-based inhibition motif (ITIM) in its cytoplasmic tail domain. As with PD-1, the ITIM domain in the cytoplasmic region of TIGIT is predicted to recruit tyrosine phosphatases, such as SHP-1 and SHP-2, and sub sequent de-phosphorylation of tyrosine residues within the immunoreceptor tyrosine-base activation motifs (ITAM) on T cell receptor (TCR) subunits. Hence, ligation of TIGIT by receptor-ligands CD155 and CD112 expressed by tumor cells or TAMS may contribute to the suppression of TCR-signaling and T cell activation, which is essential for mounting effective anti-tumor immunity. Thus, an antagonist antibody specific for TIGIT could inhibit the CD155 and CD112 induced suppression of T cell responses and enhance anti-tumor immunity.

PD-1 is recognized as an important player in immune regulation and the maintenance of peripheral tolerance. Immune checkpoint therapies targeting PD-1 or its ligand (e.g., PD-L1) have resulted in groundbreaking improvements in clinical response in multiple human cancer types (Brahmer et al., N Engl J Med, 366: 2455-2465 (2012); Garon et al., N Engl J Med, 372:2018-2028 (2015); Hamid et al., N Engl J Med, 369:134-144(2013); Robert et al., Lancet, 384:1109-1117 (2014); Robert et al., N Engl J Med, 372: 2521-2532(2015); Robert et al., N Engl J Med, 372:320-330(2015); Topalian et al., N Engl J Med, 366:2443-2454 (2012); Topalian et al., J Clin Oncol, 32:1020-1030 (2014); Wolchok et al., N Engl J Med, 369:122-133 (2013)). Immune therapies targeting the PD-1 axis include monoclonal antibodies directed to the PD-1 receptor (e.g., KEYTRUDA® (pembrolizumab), Merck and Co., Inc., Kenilworth, NJ; OPDIVO® (nivolumab), Bristol-Myers Squibb Company, Princeton, NJ) and those that bind to the PD-L1 ligand (e.g., TECENTRIQ® (atezolizumab), Genentech, San Francisco, CA).

Intratumoral hypoxia is a driving force in cancer progression and is closely linked to poor patient prognosis and resistance to chemotherapy and radiation treatment. Hypoxia-Inducible Factors (e.g., HIF-1α and HIF-2α) are transcription factors that play central roles in the hypoxic response pathway. Under normoxic conditions, the tumor suppressor von Hippel-Lindau (VHL) protein binds to specific hydroxylated proline residues and recruits the E3 ubiquition-ligase complex that targets HIF-α proteins for proteasomal degradation. Under hypoxic conditions, HIF-α proteins accumulate and enter the nucleus to stimulate the expression of genes that regulate anaerobic metabolism, angiogenesis, cell proliferation, cell survival, extracellular matrix remodeling, pH homeostasis, amino acid and nucleotide metabolism, and genomic instability. VHL deficiency can also result in accumulated HIF expression under oxygenated conditions (pseudohypoxic conditions). Accordingly, directly targeting HIF-α proteins offers an exciting opportunity to attack tumors on multiple fronts (Keith, et al., Nature Rev. Cancer 12: 9-22, 2012).

Specifically, HIF-2α is a key oncogenic driver in clear cell renal cell carcinoma (ccRCC) (Kondo, K., et al., Cancer Cell, 1:237-246 (2002); Maranchie, J. et al, Cancer Cell, 1:247-255 (2002); Kondo, K., et al., PLoS Biol., 1:439-444 (2003)). In mouse ccRCC tumor models, knockdown of HIF-2α expression in pVHL (von Hippel-Lindau protein) defective cell lines blocked tumor growth comparable to reintroduction of pVHL. In addition, expression of a stabilized variant of HIF-2α was able to overcome the tumor suppressive role of pVHL. Belzutifan or a pharmaceutically acceptable salt thereof, a novel HIF-2α inhibitor with excellent in vitro potency, pharmacokinetic profile and in vivo efficacy in mouse models, has shown encouraging outcomes in patients with advanced renal cell carcinoma (Xu, Rui, et al., J. Med. Chem. 62:6876-6893 (2019).

HIF-2α has emerged as a key HIF isoform that is essential for von Hippel Lindau (VHL) deficient ccRCC. In VHL deficient ccRCC xenograft mouse tumor models, knockdown of HIF-2a expression inhibits tumor formation comparable with reintroduction of functional VHL and overexpression of HIF2α alone can rescue the tumor-suppressive effect of VHL (see, Kondo K., Klco J, Nakamura E, Lechpammer M, Kaelin W G Jr. Inhibition of HIF is necessary for tumor suppression by the von Hippel-Lindau protein. Cancer Cell 2002; 1:237-46; Maranchie J K, Vasselli J R, Riss J, Bonifacino J S, Linehan W M, Klausner R D. The contribution of VHL substrate binding and HIF1-alpha to the phenotype of VHL loss in renal cell carcinoma. Cancer Cell 2002; 1:247-55; Kondo K, Kim W Y, Lechpammer M, Kaelin W G Jr. Inhibition of HIF-2-alpha is sufficient to suppress pVHL-defective tumor growth. PLoS Biol 2003; 1:E83; Zimmer M, Doucette D, Siddiqui N, Iliopoulos O. Inhibition of hypoxia-inducible factor is sufficient for growth suppression of VHL−/− tumors. Mol Cancer Res 2004; 2:89-95). These data suggest that HIF-2α may be the tumorigenic driver in ccRCC. HIF proteins can also be activated in many other types of cancers due to the tumor hypoxic microenvironment and have been implicated in cancer initiation, progression and metastasis (see, Jarman E J, Ward C, Turnbull A K, Martinez-Perez C, Meehan J, Xintaropoulou C, Sims A H, Langdon S P. HER2 regulates HIF-2α and drives an increased hypoxic response in breast cancer. Breast Cancer Res. 2019 Jan. 22; 21(1):10; Wigerup C, Pahlman S., Bexell D. Therapeutic targeting of hypoxia and hypoxia-inducible factors in cancer. Pharmacology & Therapeutics 164 (2016) 152-169). HIF-2α has been shown to be stabilized by host tumor cells under tumor hypoxia, including endothelial cells, perivascular tumor cells, and immune suppressive cell types such as tumor associated macrophages (TAMs) where it plays a role in regulating innate immunity (see, Imtiyaz H Z, Williams E P, Hickey M M, Patel S A, Durham A C, Yuan L J, Hammond R, Gimotty P A, Keith B, Simon M C. Hypoxia-inducible factor 2alpha regulates macrophage function in mouse models of acute and tumor inflammation. J Clin Invest. 2010 August; 120(8):2699-714). However, little is known about which VHL proficient tumor types and combination strategies make theoretical sense to explore with an inhibitor of HIF-2α.

Von-Hippel Lindau disease (VHL disease) is an autosomal dominant syndrome that not only predisposes patients to kidney cancer (˜70% lifetime risk), but also to hemangioblastomas, pheochromocytoma and pancreatic neuroendocrine tumors. VHL disease results in tumors with constitutively active HIF-α proteins with the majority of these dependent on HIF-2α activity (Maher, et al. Eur. J. Hum. Genet. 19: 617-623, 2011). HIF-2α has been linked to cancers of the retina, adrenal gland and pancreas through both VHL disease and activating mutations. Gain-of-function HIF-2α mutations have been identified in erythrocytosis and paraganglioma with polycythemia (Zhuang, et al. NEJM 367: 922-930, 2012; Percy, et al. NEJM 358: 162-168, 2008; and Percy, et al. Am. J. Hematol. 87: 439-442, 2012). Notably, a number of known HIF-2α-target gene products (e.g., VEGF, PDGF, and cyclin D1) have been shown to play pivotal roles in cancers derived from kidney, liver, colon, lung, and brain. In fact, therapies targeted against one of the key HIF-2α regulated gene products, VEGF, have been approved for the treatment of these cancers.

3-[(1S,2S,3R)-2,3-Difluoro-1-hydroxy-7-methylsulfonyl-indan-4-yl]oxy-5-fluoro-benzonitrile (hereinafter, belzutifan), a novel HIF-2α inhibitor recently received U.S. Food and Drug Administration approval for the treatment of adult patients with von Hippel-Lindau (VHL) disease who require therapy for associated renal cell carcinoma (RCC), central nervous system (CNS) hemangioblastomas, or pancreatic neuroendocrine tumors (pNET), not requiring immediate surgery.

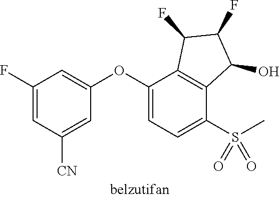 [SMILES: CS(=O)(=O)c1ccc(Oc2cc(F)cc(C#N)c2)c2c1[C@H](O)[C@H](F)[C@@H]2F]

In a recent clinical study, 49% of patients with RCC associated with VHL disease who received belzutifan had a confirmed objective response; most patients had a reduction in renal tumor size. In addition, 30% of patients with CNS hemangioblastomas had a response after treatment with belzutifan and 91% of patients with pancreatic neuroendocrine tumors responded after treatment. Belzutifan could serve as an alternative therapy or complement surgical treatment in patients afflicted with VHL disease. Investigators hypothesize that belzutifan might delay or obviate the need for serial surgeries that can burden such patients with substantial complications. (Jonasch, E. et al., N Engl J Med 2021; 385:2036-2046).

### SUMMARY

The present disclosure provides methods, pharmaceutical compositions, uses and kits of treating a cancer, or von-Hippel Lindau disease using a combination of therapeutic agents, e.g., a combination of antibodies or antigen binding fragments thereof.

The present disclosure provides methods of treating a cancer, or von-Hippel Lindau disease using a combination of: (i) a TIGIT antagonist (e.g., antibody (e.g., monoclonal antibody) or antigen binding fragment thereof), (ii) a PD-1 antagonist (e.g., antibody (e.g., monoclonal antibody) or antigen binding fragment thereof), and (iii) an HIF-2α inhibitor (e.g., antibody (e.g., monoclonal antibody) or antigen binding fragment thereof).

Among other things, the present disclosure encompasses insights that certain combinations of immune checkpoint inhibitors (e.g., a TIGIT antagonist and a PD-1 antagonist) in combination with an HIF-2α inhibitor as provided herein may enhance the efficacy without significant added toxicity as compared with existing treatments. While it has been proposed that the efficacy of anti-TIGIT antagonistic antibodies and anti-PD-1 antagonistic antibodies might be enhanced if administered in combination with other approved or experimental cancer therapies, there are no clear guidelines as to which agent combined with the anti-TIGIT antagonistic antibodies and anti-PD-1 antagonistic antibodies may be effective or in which patients the combination may enhance the efficacy of treatment.

The present disclosure provides methods of treating cancer (e.g., RCC, etc.) using a combination of a TIGIT antagonist, a PD-1 antagonist, and an HIF-2α inhibitor (e.g., belzutifan or a pharmaceutically acceptable salt thereof).

The present disclosure further provides kits including a TIGIT antagonist, a PD-1 antagonist, and an HIF-2α inhibitor (e.g., belzutifan or a pharmaceutically acceptable salt thereof).

Also provided herein are uses of a therapeutic combination for treating cancer (e.g., RCC), wherein the therapeutic combination includes a TIGIT antagonist, a PD-1 antagonist, and an HIF-2α inhibitor (e.g., belzutifan or a pharmaceutically acceptable salt thereof).

In one aspect, provided herein is a method of treating cancer, comprising administering to a human patient in need thereof.
(a) a TIGIT antagonist;
(b) a PD-1 antagonist; and
(c) belzutifan.

In one aspect, provided herein are therapeutic combinations for use in treating cancer, comprising administering to a human patient in need thereof.
(a) a TIGIT antagonist;
(b) a PD-1 antagonist; and
(c) belzutifan.

In some embodiments, the cancer is selected from the group consisting of osteosarcoma, rhabdomyosarcoma, neuroblastoma, kidney cancer, leukemia, renal transitional cell cancer, bladder cancer, Wilm's cancer, ovarian cancer, pancreatic cancer, breast cancer, prostate cancer, bone cancer, lung cancer (e.g., non-small cell lung cancer), gastric cancer, colorectal cancer, cervical cancer, synovial sarcoma, head and neck cancer, squamous cell carcinoma, lymphoma (e.g., diffuse large B-cell lymphoma (DLBCL) or non-Hodgkin lymphoma (NHL)), multiple myeloma, renal cell cancer, retinoblastoma, hepatoblastoma, hepatocellular carcinoma, melanoma, rhabdoid tumor of the kidney, Ewing's sarcoma, chondrosarcoma, brain cancer, glioblastoma, meningioma, pituitary adenoma, vestibular schwannoma, primitive neuroectodermal tumor, medulloblastoma, astrocytoma, anaplastic astrocytoma, oligodendroglioma, ependymoma, choroid plexus papilloma, polycythemia vera, thrombocythemia, idiopathic myelfibrosis, soft tissue sarcoma, thyroid cancer, endometrial cancer, and carcinoid cancer.

In some embodiments, the cancer is selected from the group consisting of bladder cancer, breast cancer, colorectal cancer (CRC), esophageal cancer, gastrointestinal cancer, hepatocellular carcinoma (HCC), melanoma, non-small cell lung cancer (NSCLC), ovarian cancer, prostate cancer, and renal cell carcinoma (RCC).

In certain embodiments, the cancer is metastatic. In some embodiments, the cancer is relapsed. In other embodiments, the cancer is refractory. In yet other embodiments, the cancer is relapsed and refractory.

In some embodiments, the cancer is bladder cancer. In some embodiments, the cancer is breast cancer, or colorectal cancer (CRC). In some embodiments, the cancer is esophageal cancer. In some embodiments, the cancer is gastrointestinal cancer. In some embodiments, the cancer is hepatocellular carcinoma (HCC). In some embodiments, the cancer is melanoma. In some embodiments, the cancer is non-small cell lung cancer (NSCLC). In some embodiments, the cancer is ovarian cancer. In some embodiments, the cancer is prostate cancer. In some embodiments, the cancer is renal cell carcinoma (RCC).

In one embodiment, the cancer is advanced RCC. In another embodiment, the cancer is advanced RCC with clear cell component. In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

Provided herein are uses of a therapeutic combination for treating von-Hippel Lindau disease, wherein the therapeutic combination includes a TIGIT antagonist, a PD-1 antagonist, and an HIF-2α inhibitor (e.g., belzutifan or a pharmaceutically acceptable salt thereof).

In one aspect, provided herein is a method of treating von-Hippel Lindau disease, comprising administering to a human patient in need thereof:
(a) a TIGIT antagonist;
(b) a PD-1 antagonist; and
(c) belzutifan.

In one aspect, provided herein are therapeutic combinations for use in treating von-Hippel Lindau disease, comprising administering to a human patient in need thereof:
(a) a TIGIT antagonist;
(b) a PD-1 antagonist; and
(c) belzutifan.

In another aspect, provided herein is a kit comprising:
(a) a TIGIT antagonist;
(b) a PD-1 antagonist; and
(c) belzutifan.

In certain embodiments, the kit further comprises instructions for administering to a human patient the TIGIT antagonist, the PD-1 antagonist, and an HIF-2α inhibitor (e.g., belzutifan or a pharmaceutically acceptable salt thereof).

In still another aspect, provided herein is use of a therapeutic combination for treating cancer in a human patient, wherein the therapeutic combination comprises:
(a) a TIGIT antagonist;
(b) a PD-1 antagonist; and
(c) belzutifan.

In some embodiments, the cancer is selected from the group consisting of bladder cancer, breast cancer, colorectal cancer (CRC), esophageal cancer, gastrointestinal cancer, hepatocellular carcinoma (HCC), melanoma, non-small cell lung cancer (NSCLC), ovarian cancer, prostate cancer, and renal cell carcinoma (RCC).

In one embodiment, the cancer is advanced RCC. In another embodiment, the cancer is advanced RCC with clear cell component. In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC. In certain embodiments of the methods, pharmaceutical compositions, kits, uses, or the combinations for use provided herein, the subject is a human patient.

In still another aspect, provided herein is use of a therapeutic combination for treating von-Hippel Lindau disease in a human patient, wherein the therapeutic combination comprises:
(a) a TIGIT antagonist;
(b) a PD-1 antagonist; and
(c) belzutifan.

In certain embodiments the methods, pharmaceutical compositions, kits, uses, or the combinations for use provided herein are for treating cancer or von-Hippel Lindau disease.

In certain embodiments of the methods, pharmaceutical compositions, kits, or uses provided herein, the PD-1 antagonist is an anti-human PD-1 monoclonal antibody or antigen binding fragment thereof.

In other embodiments of the methods, pharmaceutical compositions, kits, or uses provided herein, the PD-1 antagonist is an anti-human PD-L1 monoclonal antibody or antigen binding fragment thereof.

In some embodiments of the methods, pharmaceutical compositions, kits, or uses provided herein, the anti-human PD-1 monoclonal antibody is a humanized antibody.

In other embodiments of the methods, pharmaceutical compositions, kits, or uses provided herein, the anti-human PD-1 monoclonal antibody is a human antibody.

In some embodiments of the methods, pharmaceutical compositions, kits, or uses provided herein, the anti-human PD-L1 monoclonal antibody is a humanized antibody.

In other embodiments of the methods, pharmaceutical compositions, kits, or uses provided herein, the anti-human PD-L1 monoclonal antibody is a human antibody.

In certain embodiments of the methods, pharmaceutical compositions, kits, or uses provided herein, the TIGIT antagonist is an anti-human TIGIT monoclonal antibody or antigen binding fragment thereof.

In some embodiments of the methods, pharmaceutical compositions, kits, or uses provided herein, the anti-human TIGIT monoclonal antibody is a humanized antibody.

In other embodiments of the methods, pharmaceutical compositions, kits, or uses provided herein, the anti-human TIGIT monoclonal antibody is a human antibody.

In still other embodiments of the methods, pharmaceutical compositions, kits, uses provided herein, the anti-PD-1 antibody is independently selected from pembrolizumab, nivolumab, cemiplimab, sintilimab, tislelizumab, camrelizumab and toripalimab.

In one embodiment of the methods, pharmaceutical compositions, kits, or uses provided herein, the anti-human PD-1 monoclonal antibody is pembrolizumab.

In another embodiment of the methods, pharmaceutical compositions, kits, or uses provided herein, the anti-human PD-1 monoclonal antibody is nivolumab.

In another embodiment of the methods, pharmaceutical compositions, kits, or uses provided herein, the anti-human PD-1 monoclonal antibody is cemiplimab.

In yet another embodiment of the methods, pharmaceutical compositions, kits, or uses provided herein, the anti-human PD-1 monoclonal antibody or antigen binding fragment thereof is pidilizumab (U.S. Pat. No. 7,332,582).

In yet another embodiment of the methods, pharmaceutical compositions, kits, or uses provided herein, the anti-human PD-1 monoclonal antibody or antigen binding fragment thereof is AMP-514 (MedImmune LLC, Gaithersburg, MD).

In yet another embodiment of the methods, pharmaceutical compositions, kits, or uses provided herein, the anti-human PD-1 monoclonal antibody or antigen binding fragment thereof is PDR001 (U.S. Pat. No. 9,683,048).

In yet another embodiment of the methods, pharmaceutical compositions, kits, or uses provided herein, the anti-human PD-1 monoclonal antibody or antigen binding fragment thereof is BGB-A317 (U.S. Pat. No. 8,735,553).

In yet another embodiment of the methods, pharmaceutical compositions, kits, or uses provided herein, the anti-human PD-1 monoclonal antibody or antigen binding fragment thereof is MGA012 (MacroGenics, Rockville, MD).

In certain embodiments of the methods, kits, or uses provided herein, the anti-human TIGIT monoclonal antibody comprises three light chain CDRs comprising CDRL ID NO: 111, CDRL2 of SEQ ID NO: 112, and CDRL3 of SEQ ID NO: 113 and three heavy chain CDRs comprising CDRH1 of SEQ ID NO: 108, CDRH2 of SEQ ID NO: 154, and CDRH3 of SEQ ID NO: 110.

In some embodiments of the methods, kits, or uses provided herein the anti-human TIGIT monoclonal antibody or antigen binding fragment thereof comprises a heavy chain variable region comprising SEQ ID NO: 148 and a light chain variable region comprising SEQ ID NO: 152.

In other embodiments of the methods, kits, or uses provided herein, the anti-human TIGIT monoclonal antibody or antigen binding fragment thereof comprises a light chain comprising or consisting of an amino acid sequence as set forth in SEQ ID NO: 294 and a heavy chain comprising or consisting of an amino acid sequence as set forth in SEQ ID NO: 295.

In one specific embodiment of the methods, kits, or uses provided herein, the PD-1 antagonist is pembrolizumab; and the TIGIT antagonist is a monoclonal antibody or antigen binding fragment thereof comprising three light chain CDRs comprising CDRL1 of SEQ ID NO: 111, CDRL2 of SEQ ID NO: 112, and CDRL3 of SEQ ID NO: 113 and three heavy chain CDRs comprising CDRH1 of SEQ ID NO: 108, CDRH2 of SEQ ID NO: 154, and CDRH3 of SEQ ID NO: 110.

In one specific embodiment of the methods, kits, or uses provided herein, the PD-1 antagonist is nivolumab; and the TIGIT antagonist is a monoclonal antibody or antigen binding fragment thereof comprising three light chain CDRs comprising CDRL1 of SEQ ID NO: 111, CDRL2 of SEQ ID NO: 112, and CDRL3 of SEQ ID NO: 113 and three heavy chain CDRs comprising CDRH1 of SEQ ID NO: 108, CDRH2 of SEQ ID NO: 154, and CDRH3 of SEQ ID NO: 110.

In one specific embodiment of the methods, kits, or uses provided herein, the PD-1 antagonist is cemiplimab; and the TIGIT antagonist is a monoclonal antibody or antigen binding fragment thereof comprising three light chain CDRs comprising CDRL1 of SEQ ID NO: 111, CDRL2 of SEQ ID NO: 112, and CDRL3 of SEQ ID NO: 113 and three heavy chain CDRs comprising CDRH1 of SEQ ID NO: 108, CDRH2 of SEQ ID NO: 154, and CDRH3 of SEQ ID NO: 110.

In another aspect, provided herein is a method of enhancing T cell activity, comprising contacting the T cells with:
(a) an anti-human TIGIT antibody (e.g., monoclonal antibody) or antigen binding fragment thereof;
(b) an anti-human PD-1 antibody (e.g., monoclonal antibody) or antigen binding fragment thereof, and
(c) belzutifan.

In some embodiments, the enhancement of T cell activity occurs in vitro. In other embodiments, the enhancement of T cell activity occurs in vivo. For example, the enhancement is in a subject including but not limited to a human subject or human patient.

In certain embodiments, the enhancement of T cell activity is measured by increased cytokine production. In other embodiments, the enhancement of T cell activity is measured by increased cell proliferation.

In some embodiments, provided herein is a method of increasing cytokine production of T cells, comprising contacting the T cells with:
(a) an anti-human TIGIT antibody (e.g., monoclonal antibody) or antigen binding fragment thereof;
(b) an anti-human PD-1 antibody (e.g., monoclonal antibody) or antigen binding fragment thereof, and
(c) belzutifan.

In some embodiments, the increased cytokine production of T cells occurs in vitro. In other embodiments, the increased cytokine production of T cells occurs in vivo.

In some embodiments of the methods, kits, or uses described herein, the human patient is administered about 200 mg, about 240 mg, or about 2 mg/kg pembrolizumab, and pembrolizumab is administered once every three weeks. In one embodiment, the human patient is administered about 200 mg pembrolizumab once every three weeks. In one embodiment, the human patient is administered about 240 mg pembrolizumab once every three weeks. In one embodiment, the human patient is administered about 2 mg/kg pembrolizumab once every three weeks.

In certain embodiments of the methods, kits, or uses described herein, the human patient is administered about 400 mg pembrolizumab, and pembrolizumab is administered once every six weeks.

In other embodiments of the methods, kits, or uses described herein, the human patient is administered about 240 mg or about 3 mg/kg nivolumab once every two weeks, or about 480 mg nivolumab once every four weeks. In one specific embodiment, the human patient is administered about 240 mg nivolumab once every two weeks. In one specific embodiment, the human patient is administered about 3 mg/kg nivolumab once every two weeks. In one specific embodiment, the human patient is administered about 480 mg nivolumab once every four weeks.

In yet other embodiments of the methods, kits, or uses described herein, the human patient is administered about 350 mg cemiplimab, and cemiplimab is administered once every three weeks.

In some embodiments of the methods, kits, or uses described herein, the human patient is administered about 200 mg, about 240 mg, or about 2 mg/kg of the anti-human TIGIT monoclonal antibody or antigen binding fragment thereof comprising a light chain comprising or consisting of an amino acid sequence as set forth in SEQ ID NO: 294 and a heavy chain comprising or consisting of an amino acid sequence as set forth in SEQ ID NO: 295, and the anti-human TIGIT monoclonal antibody or antigen binding fragment thereof is administered once every three weeks. In one embodiment, the human patient is administered about 200 mg of the anti-human TIGIT monoclonal antibody or antigen binding fragment thereof once every three weeks. In one embodiment, the human patient is administered about 240 mg of the anti-human TIGIT monoclonal antibody or antigen binding fragment thereof once every three weeks. In one embodiment, the human patient is administered about 2 mg/kg of the anti-human TIGIT monoclonal antibody or antigen binding fragment thereof once every three weeks.

In certain embodiments of the methods described herein, the human patient is administered about 400 mg of the anti-human TIGIT monoclonal antibody or antigen binding fragment thereof comprising a light chain comprising or consisting of an amino acid sequence as set forth in SEQ ID NO: 294 and a heavy chain comprising or consisting of an amino acid sequence as set forth in SEQ ID NO: 295, and the anti-human TIGIT monoclonal antibody or antigen binding fragment thereof is administered once every six weeks.

In certain embodiments of the methods described herein, the human patient is administered about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg belzutifan once a day.

Thus, in some embodiments, the human patient is administered:
(a) about 200 mg, about 240 mg, or about 2 mg/kg pembrolizumab once every three or six weeks;
(b) about 200 mg, about 240 mg, or about 2 mg/kg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 40 mg, about 80 mg, or about 120 mg belzutifan once a day.

In certain embodiments, the human patient is administered:
(a) about 200 mg pembrolizumab once every three weeks;
(b) about 200 mg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 40 mg, about 80 mg, or about 120 mg belzutifan once a day.

In certain embodiments, the human patient is administered:
(a) about 240 mg pembrolizumab once every three weeks;
(b) about 240 mg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 40 mg, about 80 mg, or about 120 mg belzutifan once a day.

In certain embodiments, the human patient is administered:
(a) about 2 mg/kg pembrolizumab once every three weeks;
(b) about 2 mg/kg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 40 mg, about 80 mg, or about 120 mg belzutifan once a day.

In certain embodiments, the human patient is administered:
(a) about 400 mg pembrolizumab once every six weeks;
(b) about 400 mg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every six weeks; and
(c) about 40 mg, about 80 mg, or about 120 mg belzutifan once a day.

In a specific embodiment, provided herein is a method of treating RCC, comprising administering to a human patient in need thereof:
(a) about 200 mg pembrolizumab once every three weeks;
(b) about 200 mg of an anti-human TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks; and
(c) about 40 mg, about 80 mg, or about 120 mg belzutifan once a day.

In certain embodiments of the methods, kits and uses provided herein, the anti-human TIGIT monoclonal antibody and the anti-human PD-1 monoclonal antibody are administered on the same day. In some embodiments, the anti-human TIGIT monoclonal antibody and the anti-human PD-1 monoclonal antibody are administered sequentially. In some embodiments, the anti-human TIGIT monoclonal antibody and the anti-human PD-1 monoclonal antibody are administered concurrently. In some embodiments, the anti-human TIGIT monoclonal antibody and the anti-human PD-1 monoclonal antibody are co-formulated.

### BRIEF DESCRIPTION OF THE FIGURES

FIG. 1 shows anti-tumor effects of concurrent administration of a PD-1 antagonist, a TIGIT antagonist, and belzutifan by average tumor volumes in each treatment group. Lines are only depicted until at least half animals in the group remained.

FIG. 2 shows anti-tumor effects of concurrent administration of a PD-1 antagonist, a TIGIT antagonist, and belzutifan as a waterfall plot depicting the best overall response shown as percentage change in tumor volume at end of study, Day 43.

FIG. 3 depicts comparison of final tumor volumes at Day 43 from the combination of anti-PD-1 antibody and anti-TIGIT antibody versus the combination of anti-PD-1 antibody, anti-TIGIT antibody and belzutifan.

### DETAILED DESCRIPTION OF THE INVENTION

### Definitions

Certain technical and scientific terms are specifically defined below. Unless specifically defined elsewhere in this document, all other technical and scientific terms used herein have the meaning commonly understood by one of ordinary skill in the art to which this disclosure relates.

“About” when used to modify a numerically defined parameter (e.g., the dose of an anti-TIGIT antibody or antigen binding fragment thereof, an anti-PD-1 antibody or antigen binding fragment thereof, or an HIF-2α inhibitor (e.g., belzutifan or a pharmaceutically acceptable salt thereof), or the length of treatment time with a combination therapy described herein) means that the parameter is within 20%, within 15%, within 10%, within 9%, within 8%, within 7%, within 6%, within 5%, within 4%, within 3%, within 2%, within 1%, or less of the stated numerical value or range for that parameter; where appropriate, the stated parameter may be rounded to the nearest whole number. For example, a dose of about 5 mg/kg may vary between 4.5 mg/kg and 5.5 mg/kg.

As used herein, including the appended claims, the singular forms of words such as “a,” “an,” and “the,” include their corresponding plural references unless the context clearly dictates otherwise.

The terms “administration” or “administer” refers to the act of injecting or otherwise physically delivering a substance as it exists outside the body (e.g., an anti-TIGIT antibody, an anti-PD-1 antibody, and an HIF-2α inhibitor (e.g., belzutifan or a pharmaceutically acceptable salt thereof) as described herein) into a patient, such as by oral, mucosal, intradermal, intravenous, subcutaneous, intramuscular delivery, and/or any other methods of physical delivery described herein or known in the art.

As used herein, the term “antibody” refers to any form of immunoglobulin molecule that exhibits the desired biological or binding activity. Thus, it is used in the broadest sense and specifically covers, but is not limited to, monoclonal antibodies (including full length monoclonal antibodies), polyclonal antibodies, multispecific antibodies (e.g., bispecific antibodies), humanized, fully human antibodies, and chimeric antibodies. “Parental antibodies” are antibodies obtained by exposure of an immune system to an antigen prior to modification of the antibodies for an intended use, such as humanization of an antibody for use as a human therapeutic. As used herein, the term “antibody” encompasses not only intact polyclonal or monoclonal antibodies, but also, unless otherwise specified, fusion proteins comprising an antigen binding fragment thereof that competes with the intact antibody for specific.

In general, the basic antibody structural unit comprises a tetramer. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The variable regions of each light/heavy chain pair form the antibody binding site. Thus, in general, an intact antibody has two binding sites. The carboxy-terminal portion of the heavy chain may define a constant region primarily responsible for effector function. Typically, human light chains are classified as kappa and lambda light chains. Furthermore, human heavy chains are typically classified as mu, delta, gamma, alpha, or epsilon, and define the antibody's isotype as IgM, IgD, IgG, IgA, and IgE, respectively. Within light and heavy chains, the variable and constant regions are joined by a “J” region of about 12 or more amino acids, with the heavy chain also including a “D” region of about 10 more amino acids. See generally, Fundamental Immunology Ch. 7 (Paul, W., ed., 2nd ed. Raven Press, N.Y. (1989).

“Variable regions” or “V region” or “V chain” as used herein means the segment of IgG chains which is variable in sequence between different antibodies. A “variable region” of an antibody refers to the variable region of the antibody light chain or the variable region of the antibody heavy chain, either alone or in combination. The variable region of the heavy chain may be referred to as “V_{H}.” The variable region of the light chain may be referred to as “V_{L}.”

Typically, the variable regions of both the heavy and light chains comprise three hypervariable regions, also called complementarity determining regions (CDRs), which are located within relatively conserved framework regions (FR). The CDRs are usually aligned by the framework regions, enabling binding to a specific epitope. In general, from N-terminal to C-terminal, both light and heavy chains variable domains comprise FR1, CDR1, FR2, CDR2, FR3, CDR3, and FR4. As referred to herein the light chain CDRs are CDRL1, CDRL2 and CDRL3, respectively, and the heavy chain CDRs are CDRH1, CDRH2 and CDRH3, respectively. The assignment of amino acids to each domain is, generally, in accordance with the definitions of Sequences of Proteins of Immunological Interest, Kabat, et al.; National Institutes of Health, Bethesda, Md.; 5th ed.; NIH Publ. No. 91-3242 (1991); Kabat (1978) Adv. Prot. Chem. 32:1-75; Kabat, et al., (1977) J. Biol. Chem. 252:6609-6616; Chothia, et al., (1987) J Mol. Biol. 196:901-917 or Chothia, et al., (1989) Nature 342:878-883.

A “CDR” refers to one of three hypervariable regions (H1, H2, or H3) within the non-framework region of the antibody V_{H} β-sheet framework, or one of three hypervariable regions (L1, L2, or L3) within the non-framework region of the antibody V_{L} β-sheet framework. Accordingly, CDRs are variable region sequences interspersed within the framework region sequences. CDR regions are well known to those skilled in the art and have been defined by, for example, Kabat as the regions of most hypervariability within the antibody variable domains. CDR region sequences also have been defined structurally by Chothia as those residues that are not part of the conserved b-sheet framework, and thus are able to adapt to different conformation. Both terminologies are well recognized in the art. CDR region sequences have also been defined by AbM, Contact, and IMGT. The positions of CDRs within a canonical antibody variable region have been determined by comparison of numerous structures (Al-Lazikani et al., 1997, J. Mol. Biol. 273:927-48; Morea et al., 2000, Methods 20:267-79). Because the number of residues within a hypervariable region varies in different antibodies, additional residues relative to the canonical positions are conventionally numbered with a, b, c and so forth next to the residue number in the canonical variable region numbering scheme (Al-Lazikani et al., supra). Such nomenclature is similarly well known to those skilled in the art. Correspondence between the numbering system, including, for example, the Kabat numbering and the IMGT unique numbering system, is well known to one skilled in the art and shown below in Table 1. In some embodiments, the CDRs are as defined by the Kabat numbering system. In other embodiments, the CDRs are as defined by the IMGT numbering system. In yet other embodiments, the CDRs are as defined by the AbM numbering system. In still other embodiments, the CDRs are as defined by the Chothia numbering system. In yet other embodiments, the CDRs are as defined by the Contact numbering system.

TABLE 1
Correspondence between the CDR Numbering Systems

| | Kabat + | | | | | |
|---|---|---|---|---|---|---|
| | Chothia | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52-56 | 47-58 |
| V_{H} CDR3 | 95-102 | 105-117 | 95-102 | 95-102 | 95-102 | 93-101 |
| V_{L} CDR1 | 24-34 | 27-38 | 24-34 | 24-34 | 24-34 | 30-36 |
| V_{L} CDR2 | 50-56 | 56-65 | 50-56 | 50-56 | 50-56 | 46-55 |
| V_{L} CDR3 | 89-97 | 105-117 | 89-97 | 89-97 | 89-97 | 89-96 |

“Chimeric antibody” refers to an antibody in which a portion of the heavy and/or light chain contains sequences derived from a particular species (e.g., human) or belonging to a particular antibody class or subclass, while the remainder of the chain(s) is derived from another species (e.g., mouse) or belonging to another antibody class or subclass, as well as fragments of such antibodies, so long as they exhibit the desired biological activity.

“Human antibody” refers to an antibody that comprises human immunoglobulin protein sequences or derivatives thereof. A human antibody may contain murine carbohydrate chains if produced in a mouse, in a mouse cell, or in a hybridoma derived from a mouse cell. Similarly, “mouse antibody” or “rat antibody” refer to an antibody that comprises only mouse or rat immunoglobulin sequences or derivatives thereof, respectively.

“Humanized antibody” refers to forms of antibodies that contain sequences from non-human (e.g., murine) antibodies as well as human antibodies. Such antibodies contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. The prefix “hum”, “hu” or “h” may be added to antibody clone designations when necessary to distinguish humanized antibodies from parental rodent antibodies. The humanized forms of rodent antibodies will generally comprise the same CDR sequences of the parental rodent antibodies, although certain amino acid substitutions may be included to increase affinity, increase stability of the humanized antibody, or for other reasons.

“Monoclonal antibody” or “mAb” or “Mab”, as used herein, refers to a population of substantially homogeneous antibodies, i.e., the antibody molecules comprising the population are identical in amino acid sequence except for possible naturally occurring mutations that may be present in minor amounts. In contrast, conventional (polyclonal) antibody preparations typically include a multitude of different antibodies having different amino acid sequences in their variable domains, particularly their CDRs, which are often specific for different epitopes. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method first described by Kohler et al. (1975) Nature 256: 495, or may be made by recombinant DNA methods (see, e.g., U.S. Pat. No. 4,816,567). The “monoclonal antibodies” may also be isolated from phage antibody libraries using the techniques described in Clackson et al. (1991) Nature 352: 624-628 and Marks et al. (1991) J. Mol. Biol. 222: 581-597, for example. See also Presta (2005) J. Allergy Clin. Immunol. 116:731.

As used herein, unless otherwise indicated, “antibody fragment” or “antigen binding fragment” refers to a fragment of an antibody that retains the ability to bind specifically to the antigen, e.g., fragments that retain one or more CDR regions and the ability to bind specifically to the antigen. An antibody that “specifically binds to” TIGIT or PD-1 is an antibody that exhibits preferential binding to TIGIT or PD-1 (as appropriate) as compared to other proteins, but this specificity does not require absolute binding specificity. An antibody is considered “specific” for its intended target if its binding is determinative of the presence of the target protein in a sample, e.g., without producing undesired results such as “false” positives. Antibodies, or binding fragments thereof, will bind to the target protein with an affinity that is at least two-fold greater, preferably at least ten times greater, more preferably at least 20-times greater, and most preferably at least 100-times greater than the affinity with non-target proteins.

Antigen binding portions include, for example, Fab, Fab′, F(ab′)2, Fd, Fv, fragments including CDRs, and single chain variable fragment antibodies (scFv), and polypeptides that contain at least a portion of an immunoglobulin that is sufficient to confer specific antigen binding to the antigen (e.g., TIGIT or PD-1). An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1, and IgA2. The heavy-chain constant regions that correspond to the different classes of immunoglobulins are called alpha, delta, epsilon, gamma, and mu, respectively. The subunit structures and three-dimensional configurations of different classes of immunoglobulins are well known.

As used herein, the terms “at least one” item or “one or more” item each include a single item selected from the list as well as mixtures of two or more items selected from the list.

As used herein, the term “immune response” relates to any one or more of the following: specific immune response, non-specific immune response, both specific and non-specific response, innate response, primary immune response, adaptive immunity, secondary immune response, memory immune response, immune cell activation, immune cell-proliferation, immune cell differentiation, and cytokine expression.

As used herein, the term “HIF-2α inhibitor” means any chemical compound or biological molecule that inhibits the activity of HIF-2α. Alternative names or synonyms for HIF-2α include but are not limited to: hypoxia-inducible factor-2alpha, endothelial PAS domain-containing protein 1, and EPAS1.

The term “subject” (alternatively “patient”) as used herein refers to a mammal that has been the object of treatment, observation, or experiment. The mammal may be male or female. The mammal may be one or more selected from the group consisting of humans, bovine (e.g., cows), porcine (e.g., pigs), ovine (e.g., sheep), capra (e.g., goats), equine (e.g., horses), canine (e.g., domestic dogs), feline (e.g., house cats), lagomorph (e.g., rabbits), rodent (e.g., rats or mice), Procyon lotor (e.g., raccoons). In particular embodiments, the subject is human.

The term “subject in need thereof” as used herein refers to a subject diagnosed with or suspected of having cancer or an infectious disease as defined herein.

The therapeutic agents and compositions provided by the present disclosure can be administered via any suitable enteral route or parenteral route of administration. The term “enteral route” of administration refers to the administration via any part of the gastrointestinal tract. Examples of enteral routes include oral, mucosal, buccal, and rectal route, or intragastric route. “Parenteral route” of administration refers to a route of administration other than enteral route. Examples of parenteral routes of administration include intravenous, intramuscular, intradermal, intraperitoneal, intratumor, intravesical, intraarterial, intrathecal, intracapsular, intraorbital, intracardiac, transtracheal, intraarticular, subcapsular, subarachnoid, intraspinal, epidural and intrasternal, subcutaneous, or topical administration. The therapeutic agents and compositions of the disclosure can be administered using any suitable method, such as by oral ingestion, nasogastric tube, gastrostomy tube, injection, infusion, implantable infusion pump, and osmotic pump. The suitable route and method of administration may vary depending on a number of factors such as the specific therapeutic agent being used, the rate of absorption desired, specific formulation or dosage form used, type or severity of the disorder being treated, the specific site of action, and conditions of the patient, and can be readily selected by a person skilled in the art.

The term “variant” when used in relation to an antibody (e.g., an anti-TIGIT antibody or an anti-PD-1 antibody) or an amino acid region within the antibody may refer to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a variant of an anti-PD-1 antibody may result from one or more (such as, for example, about 1 to about 25, about 1 to about 20, about 1 to about 15, about 1 to about 10, or about 1 to about 5) changes to an amino acid sequence of a native or previously unmodified anti-PD-1 antibody. Variants may be naturally occurring or may be artificially constructed. Polypeptide variants may be prepared from the corresponding nucleic acid molecules encoding the variants. In specific embodiments, an antibody variant (e.g., an anti-TIGIT antibody variant or an anti-PD-1 antibody variant) at least retains the antibody functional activity. In specific embodiments, an anti-TIGIT antibody variant binds to TIGIT and/or is antagonistic to TIGIT activity. In some embodiments, an anti-PD-1 antibody variant binds to PD-1 and/or is antagonistic to PD-1 activity.

“Conservatively modified variants” or “conservative substitution” refers to substitutions of amino acids in a protein with other amino acids having similar characteristics (e.g., charge, side-chain size, hydrophobicity/hydrophilicity, backbone conformation and rigidity, etc.), such that the changes can frequently be made without altering the biological activity or other desired property of the protein, such as antigen affinity and/or specificity. Those of skill in this art recognize that, in general, single amino acid substitutions in non-essential regions of a polypeptide do not substantially alter biological activity (see, e.g., Watson et al. (1987) Molecular Biology of the Gene, The Benjamin/Cummings Pub. Co., p. 224 (4th Ed.)). In addition, substitutions of structurally or functionally similar amino acids are less likely to disrupt biological activity. Exemplary conservative substitutions are set forth in Table 2 below.

TABLE 2
Exemplary Conservative Amino Acid Substitutions

| Original residue | Conservative substitution |
|---|---|
| Ala (A) | Gly; Ser |
| Arg (R) | Lys; His |
| Asn (N) | Gln; His |
| Asp (D) | Glu; Asn |
| Cys (C) | Ser; Ala |
| Gln (Q) | Asn |
| Glu (E) | Asp; Gln |
| Gly (G) | Ala |
| His (H) | Asn; Gln |
| Ile (I) | Leu; Val |
| Leu (L) | Ile; Val |
| Lys (K) | Arg; His |
| Met (M) | Leu; Ile; Tyr |
| Phe (F) | Tyr; Met; Leu |
| Pro (P) | Ala |
| Ser (S) | Thr |
| Thr (T) | Ser |
| Trp (W) | Tyr; Phe |
| Tyr (Y) | Trp; Phe |
| Val (V) | Ile; Leu |

“Homology” refers to sequence similarity between two polypeptide sequences when they are optimally aligned. When a position in both of the two compared sequences is occupied by the same amino acid monomer subunit, e.g., if a position in a light chain CDR of two different Abs is occupied by alanine, then the two Abs are homologous at that position. The percent of homology is the number of homologous positions shared by the two sequences divided by the total number of positions compared×100. For example, if 8 of 10 of the positions in two sequences are matched when the sequences are optimally aligned then the two sequences are 80% homologous. Generally, the comparison is made when two sequences are aligned to give maximum percent homology. For example, the comparison can be performed by a BLAST algorithm wherein the parameters of the algorithm are selected to give the largest match between the respective sequences over the entire length of the respective reference sequences.

The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

“RECIST 1.1 Response Criteria” as used herein means the definitions set forth in Eisenhauer, E. A. et al., Eur. J. Cancer 45:228-247 (2009) for target lesions or nontarget lesions, as appropriate based on the context in which response is being measured.

“Sustained response” means a sustained therapeutic effect after cessation of treatment as described herein. In some embodiments, the sustained response has a duration that is at least the same as the treatment duration, or at least 1.5, 2.0, 2.5 or 3 times longer than the treatment duration.

As used herein, the term “treat” or “treating” means to administer a therapeutic combination of a TIGIT antagonist, a PD-1 antagonist and an HIF-2α inhibitor, such as, e.g., an anti-human PD-1 monoclonal antibody or antigen binding fragment thereof, an anti-human TIGIT monoclonal antibody or antigen binding fragment thereof, and an HIF-2α inhibitor (e.g., belzutifan or a pharmaceutically acceptable salt thereof) to a subject or patient having one or more disease symptoms as provided herein. Typically, the agents of the therapeutic combination are administered in an amount effective to alleviate one or more disease symptoms in the treated subject or population, whether by inducing the regression of or inhibiting the progression of such symptom(s) by any clinically measurable degree. The amount of the agents of the therapeutic combination that is effective to alleviate any particular disease symptom may vary according to factors such as the disease state, age, and weight of the patient, and the ability of the therapeutic combination to elicit a desired response in the subject. Whether a disease symptom has been alleviated can be assessed by any clinical measurement typically used by physicians or other skilled healthcare providers to assess the severity or progression status of that symptom.

“Treat” or “treating” cancer as used herein means to administer a therapeutic combination of a TIGIT antagonist, a PD-1 antagonist and an HIF-2α inhibitor, such as, e.g., an anti-human PD-1 monoclonal antibody or antigen binding fragment thereof, an anti-human TIGIT monoclonal antibody or antigen binding fragment thereof, and an HIF-2α inhibitor (e.g., belzutifan or a pharmaceutically acceptable salt thereof), to a subject having cancer or diagnosed with cancer to achieve at least one positive therapeutic effect, such as, for example, reduced number of cancer cells, reduced tumor size, reduced rate of cancer cell infiltration into peripheral organs, or reduced rate of tumor metastasis or tumor growth, comprising administration by oral, mucosal, intradermal, intravenous, subcutaneous, intramuscular delivery, and/or any other methods of physical delivery described herein or known in the art. “Treatment” may include one or more of the following: inducing/increasing an antitumor immune response, decreasing the number of one or more tumor markers, halting or delaying the growth of a tumor or blood cancer or progression of disease such as cancer, stabilization of disease, inhibiting the growth or survival of tumor cells, eliminating or reducing the size of one or more cancerous lesions or tumors, decreasing the level of one or more tumor markers, ameliorating or abrogating the clinical manifestations of disease, reducing the severity or duration of the clinical symptoms, prolonging the survival or patient relative to the expected survival in a similar untreated patient, and inducing complete or partial remission of a cancerous condition, wherein the disease is cancer, and in certain embodiments wherein the cancer is selected from the group consisting of endometrial cancer cervical cancer, head and neck cancer (HNSCC), biliary cancer, esophageal cancer, bladder cancer, breast cancer, triple negative breast cancer (TNBC), non-small cell lung cancer (NSCLC), colorectal cancer (CRC), renal cell carcinoma (RCC), hepatocellular carcinoma (HCC), and melanoma. The amount of a therapeutic agent that is effective to alleviate any particular disease symptom may vary according to factors such as the disease state, age, and weight of the patient, and the ability of the drug to elicit a desired response in the subject. Whether a disease symptom has been alleviated can be assessed by any clinical measurement typically used by physicians or other skilled healthcare providers to assess the severity or progression status of that symptom.

Positive therapeutic effects in cancer can be measured in a number of ways (See, W. A. Weber, J. Nucl. Med. 50:1S-10S (2009)). For example, with respect to tumor growth inhibition, according to NCI standards, a T/C≤42% is the minimum level of anti-tumor activity. A T/C<10% is considered a high anti-tumor activity level, with T/C (%)=Median tumor volume of the treated/Median tumor volume of the control×100. In some embodiments, the treatment achieved by a combination therapy of the disclosure is any of PR, CR, OR, PFS, DFS, and OS. PFS, also referred to as “Time to Tumor Progression” indicates the length of time during and after treatment that the cancer does not grow, and includes the amount of time patients have experienced a CR or PR, as well as the amount of time patients have experienced SD. DFS refers to the length of time during and after treatment that the patient remains free of disease. OS refers to a prolongation in life expectancy as compared to naive or untreated individuals or patients. In some embodiments, response to a combination therapy of the disclosure is any of PR, CR, PFS, DFS, or OR that is assessed using RECIST 1.1 response criteria. The treatment regimen for a combination therapy of the disclosure that is effective to treat a cancer patient may vary according to factors such as the disease state, age, and weight of the patient, and the ability of the therapy to elicit an anti-cancer response in the subject. While an embodiment of any of the aspects of the disclosure may not be effective in achieving a positive therapeutic effect in every subject, it should do so in a statistically significant number of subjects as determined by any statistical test known in the art such as the Student's t-test, the chi²-test, the U-test according to Mann and Whitney, the Kruskal-Wallis test (H-test), Jonckheere-Terpstra-test and the Wilcoxon-test.

“Treat” or “treating” an infectious disease or an infection as used herein means to administer a therapeutic combination of a TIGIT antagonist, a PD-1 antagonist and an HIF-2α inhibitor, such as, e.g., an anti-human PD-1 monoclonal antibody or antigen binding fragment thereof, an anti-human TIGIT monoclonal antibody or antigen binding fragment thereof, and an HIF-2α inhibitor (e.g., belzutifan or a pharmaceutically acceptable salt thereof), to a subject having an infectious disease or an infection (e.g., caused by many pathogens, including bacteria, viruses, fungi) to achieve at least one positive therapeutic effect.

The term “co-formulation” refers to a formulation comprising two or more of therapeutic agents. In some embodiments, co-formulation comprises a TIGIT antagonist, and a PD-1 antagonist.

The term “pharmaceutically acceptable carrier” refers to any inactive substance that is suitable for use in a formulation for the delivery of a therapeutic agent. A carrier may be an anti-adherent, binder, coating, disintegrant, filler or diluent, preservative (such as antioxidant, antibacterial, or antifungal agent), sweetener, absorption delaying agent, wetting agent, emulsifying agent, buffer, and the like. Examples of suitable pharmaceutically acceptable carriers include water, ethanol, polyols (such as glycerol, propylene glycol, polyethylene glycol, and the like), dextrose, vegetable oils (such as olive oil), saline, buffer, buffered saline, and isotonic agents such as sugars, polyalcohols, sorbitol, and sodium chloride.

As used herein, the terms “combination,” “combination therapy,” and “therapeutic combination” refer to treatments in which at least one TIGIT antagonist, at least one PD-1 antagonist and at least HIF-2α inhibitor, such as, e.g., an anti-human TIGIT monoclonal antibody or antigen-binding fragment thereof, an anti-human PD-1 monoclonal antibody or antigen-binding fragment thereof, and HIF-2α inhibitor (e.g., belzutifan or a pharmaceutically acceptable salt thereof), and optionally additional therapeutic agents, each are administered to a patient in a coordinated manner, over an overlapping period of time. The period of treatment with the at least one TIGIT antagonist (the “anti-TIGIT treatment”) is the period of time that a patient undergoes treatment with the TIGIT antagonist, e.g., an anti-human TIGIT monoclonal antibody (or antigen-binding fragment thereof); that is, the period of time from the initial dosing with the TIGIT antagonist through the final day of a treatment cycle. Similarly, the period of treatment with the at least one PD-1 antagonist (the “anti-PD-1 treatment”) is the period of time that a patient undergoes treatment with the PD-1 antagonist, e.g., an anti-human PD-1 monoclonal antibody (or antigen-binding fragment thereof); that is, the period of time from the initial dosing with the PD-1 antagonist through the final day of a treatment cycle. The period of treatment with the at least one HIF-2α inhibitor (the “HIF-2α inhibitor treatment”) is the period of time that a patient undergoes treatment with the HIF-2α inhibitor, e.g., belzutifan or a pharmaceutically acceptable salt thereof; that is, the period of time from the initial dosing with the HIF-2α inhibitor through the final day of a treatment cycle. In the methods, uses, and therapeutic combinations described herein, the anti-TIGIT treatment overlaps by at least one day with the anti-PD-1 treatment and overlaps by at least one day with the HIF-2α inhibitor treatment. In certain embodiments, the anti-TIGIT treatment, the anti-PD-1 treatment, and the HIF-2α inhibitor treatment are the same period of time. In some embodiments, the anti-TIGIT treatment begins prior to the anti-PD-1 and/or the HIF-2α inhibitor treatment. In other embodiments, the anti-TIGIT treatment begins after the anti-PD-1 and/or the HIF-2α inhibitor treatment. In yet other embodiments, the anti-PD-1 treatment begins prior to the anti-TIGIT and/or the HIF-2α inhibitor treatment. In still other embodiments, the anti-PD-1 treatment begins after the anti-TIGIT and/or the HIF-2α inhibitor treatment. In some embodiments, the HIF-2α inhibitor treatment begins prior to the anti-PD-1 and/or the anti-TIGIT treatment. In other embodiments, the HIF-2α inhibitor treatment begins after the anti-PD-1 and/or the anti-TIGIT treatment. In certain embodiments, the anti-TIGIT treatment is terminated prior to termination of the anti-PD-1 and/or the HIF-2α inhibitor treatment. In other embodiments, the anti-TIGIT treatment is terminated after termination of the anti-PD-1 and/or the HIF-2α inhibitor treatment. In yet other embodiments, the anti-PD-1 treatment is terminated prior to termination of the anti-TIGIT and/or the HIF-2α inhibitor treatment. In still other embodiments, the anti-PD-1 treatment is terminated after termination of the anti-TIGIT and/or the HIF-2α inhibitor treatment. In certain embodiments, the HIF-2α inhibitor treatment is terminated prior to termination of the anti-PD-1 and/or the anti-TIGIT treatment. In other embodiments, the HIF-2α inhibitor treatment is terminated after termination of the anti-PD-1 and/or the anti-TIGIT treatment.

The terms “treatment regimen,” “dosing protocol,” and “dosing regimen” are used interchangeably to refer to the dose and timing of administration of each therapeutic agent in a combination therapy of the disclosure.

The terms “cancer”, “cancerous”, or “malignant” refer to or describe the physiological condition in mammals that is typically characterized by unregulated cell growth. Examples of cancer include but are not limited to, carcinoma, lymphoma, leukemia, blastoma, and sarcoma. More particular examples of such cancers include, but are not limited to, squamous cell carcinoma, myeloma, small-cell lung cancer, non-small cell lung cancer, glioma, Hodgkin lymphoma, non-hodgkin's lymphoma, acute myeloid leukemia (AML), multiple myeloma, gastrointestinal (tract) cancer, renal cancer, ovarian cancer, liver cancer, lymphoblastic leukemia, lymphocytic leukemia, colorectal cancer, endometrial cancer, kidney cancer, prostate cancer, thyroid cancer, melanoma, chondrosarcoma, neuroblastoma, pancreatic cancer, glioblastoma multiforme, cervical cancer, brain cancer, stomach cancer, bladder cancer, hepatoma, hepatocellular carcinoma, biliary cancer, esophageal cancer, breast cancer, triple negative breast cancer, colon carcinoma, and head and neck cancer.

“Tumor” as it applies to a subject diagnosed with, or suspected of having, a cancer refers to a malignant or potentially malignant neoplasm or tissue mass of any size, and includes primary tumors and secondary neoplasms. Non-limiting examples of tumors include solid tumor (e.g., sarcoma (such as chondrosarcoma), carcinoma (such as colon carcinoma), blastoma (such as hepatoblastoma), etc.) and blood tumor (e.g., leukemia (such as acute myeloid leukemia (AMVL)), lymphoma (such as DLBCL), multiple myeloma (MM), etc.).

“Tumor burden” also referred to as “tumor load”, refers to the total amount of tumor material distributed throughout the body. Tumor burden refers to the total number of cancer cells or the total size of tumor(s), throughout the body, including lymph nodes and bone narrow. Tumor burden can be determined by a variety of methods known in the art, such as, e.g., by measuring the dimensions of tumor(s) upon removal from the subject, e.g., using calipers, or while in the body using imaging techniques, e.g., ultrasound, bone scan, computed tomography (CT) or magnetic resonance imaging (MRI) scans.

The term “tumor volume” or “tumor size” refers to the total size of the tumor which can be measured as the length and width of a tumor. Tumor size may be determined by a variety of methods known in the art, such as, e.g., by measuring the dimensions of tumor(s) upon removal from the subject, e.g., using calipers, or while in the body using imaging techniques, e.g., bone scan, ultrasound, CT or MRI scans.

As used herein, the term “effective amount” refer to an amount of a TIGIT antagonist, a PD-1 antagonist and an HIF-2α inhibitor, such as, e.g., an anti-TIGIT antibody or antigen binding fragment, an anti-PD-1 antibody or antigen binding fragment of the invention, an HIF-2α inhibitor (e.g., belzutifan or a pharmaceutically acceptable salt thereof), that, when administered alone or in combination with an additional therapeutic agent to a cell, tissue, or subject, is effective to cause a measurable improvement in one or more symptoms of an infection or a disease, for example cancer or the progression of cancer. An effective amount further refers to that amount of the antibody or fragment sufficient to result in at least partial amelioration of symptoms, e.g., tumor shrinkage or elimination, lack of tumor growth, increased survival time. When applied to an individual active ingredient administered alone, an effective dose refers to that ingredient alone. When applied to a combination, an effective dose refers to combined amounts of the active ingredients that result in the therapeutic effect, whether administered in combination, serially or simultaneously. An effective amount of a therapeutic may result in an improvement of a diagnostic measure or parameter by at least 10%; usually by at least 20%; preferably at least about 30%; more preferably at least 40%, and most preferably by at least 50%. An effective amount can also result in an improvement in a subjective measure in cases where subjective measures are used to assess disease severity. Toxicity and therapeutic efficacy of the antibodies or antigen binding fragments of the invention, administered alone or in combination with another therapeutic agent, can be determined by any number of systems or means. For example, the toxicity and therapeutic efficacy of the antibodies or antigen binding fragments or compounds of the invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index (LD50/ED50). The data obtained from these cell culture assays and animal studies can be used in formulating a range of dosage for use in human. The dosage of such compounds lies preferably within a range of circulating concentrations that include the ED50 with little or no toxicity. The dosage may vary within this range depending upon the dosage form employed and the route of administration.

It is understood that wherever embodiments are described herein with the language “comprising,” otherwise analogous embodiments described in terms of “consisting of” and/or “consisting essentially of” are also provided.

“Consists essentially of,” and variations such as “consist essentially of” or “consisting essentially of,” as used throughout the specification and claims, indicate the inclusion of any recited elements or group of elements, and the optional inclusion of other elements, of similar or different nature than the recited elements, that do not materially change the basic or novel properties of the specified dosage regimen, method, or composition.

Unless expressly stated to the contrary, all ranges cited herein are inclusive; i.e., the range includes the values for the upper and lower limits of the range as well as all values in between. As an example, temperature ranges, percentages, ranges of equivalents, and the like described herein include the upper and lower limits of the range and any value in the continuum there between. Numerical values provided herein, and the use of the term “about”, may include variations of ±1%, ±2%, ±3%, ±4%, ±5%, ±10%, ±15%, and ±20% and their numerical equivalents. All ranges also are intended to include all included sub-ranges, although not necessarily explicitly set forth. For example, a range of 3 to 7 days is intended to include 3, 4, 5, 6, and 7 days. In addition, the term “or,” as used herein, denotes alternatives that may, where appropriate, be combined; that is, the term “or” includes each listed alternative separately as well as their combination.

Where aspects or embodiments of the disclosure are described in terms of a Markush group or other grouping of alternatives, the present disclosure encompasses not only the entire group listed as a whole, but each member of the group individually and all possible subgroups of the main group, but also the main group absent one or more of the group members. The present disclosure also envisages the explicit exclusion of one or more of any of the group members in the claims.

Unless otherwise defined, all technical and scientific terms used herein have the same meaning as commonly understood by one of ordinary skill in the art to which this disclosure relates. In case of conflict, the present specification, including definitions, will control. Throughout this specification and claims, the word “comprise,” or variations such as “comprises” or“comprising” will be understood to imply the inclusion of a stated integer or group of integers but not the exclusion ofany other integer or group of integers. Unless otherwise required by context, singular terms shall include pluralities and plural terms shall include the singular. Any example(s) following the term “e.g.” or “for example” is not meant to be exhaustive or limiting.

Exemplary methods and materials are described herein, although methods and materials similar or equivalent to those described herein can also be used in the practice or testing of the present disclosure. The materials, methods, and examples are illustrative only and not intended to be limiting.

Abbreviations with Expanded Term

TABLE 3
Abbreviations

| Abbreviation | Expanded Term |
|---|---|
| 1L | First line |
| ADA | antidrug antibody |
| AB | adverse event |
| AJCC | American Joint Committee on Cancer |
| ALT | Alanine aminotransferase |
| APaT | All-Participants-as-Treated |
| aPTT | Activated partial thromboplastin time |
| BCG | Bacillus Calmette-Guerin |
| BICR | blinded independent central review |
| BP | blood pressure |
| BRAF | v-raf murine sarcoma viral oncogene homolog B1 |
| C1 | Cycle 1 |
| C2 | Cycle 2 |
| C3 | Cycle 3 |
| C4 | Cycle 4 |
| C5 | Cycle 5 |
| C6 | Cycle 6 |
| C10 | Cycle 10 |
| C14 | Cycle 14 |
| C1D1 | Cycle 1 Day 1 |
| CD | cluster of differentiation |
| CDR | Complementary determining region |
| CD28 | cluster of differentiation 28 |
| CHO | Chinese hamster ovary |
| CIV | central imaging vendor |
| CNS | central nervous system |
| CR | complete response |
| CSR | Clinical Study Report |
| CT | computed tomography |
| CTCAE | Common Terminology Criteria for Adverse Events |
| CTCAE 5.0 | Common Terminology Criteria for Adverse Events, |
| | version 5.0 |
| D 1 | Day 1 |
| D 8 | Day 8 |
| D 15 | Day 15 |
| D/C | discontinue/discontinuing |
| DLT | dose-limiting toxicity |
| DNA | deoxyribonucleic acid |
| DOR | Duration of Response |
| ECG | Electrocardiogram |
| ECHO | Echocardiogram |
| ECI | event of clinical interest |
| ECOG | Eastern Cooperative Oncology Group |
| EFS | event-free survival |
| EOT | End of treatment |
| EU | European Union |
| FR | framework region |
| HBsAg | Hepatitis B surface antigen |
| HBV | Hepatitis B virus |
| HCV | Hepatitis C virus |
| HIV | human immunodeficiency virus |
| IA | interim analysis |
| IB | Investigator's Brochure |
| IC50 | mean inhibitory concentration |
| ICF | Informed Consent Form |
| iCPD | iRECIST confirmed progressive disease |
| iCR | iRECIST complete response |
| IgG | Immunoglobulin G |
| IgG1 | Immunoglobulin G, subclass 1 |
| IMP | Investigational medicinal product |
| IND | Investigational New Drug |
| IO | immuno-oncology |
| IP | Intraperitoneal |
| iPR | iRECIST partial response |
| irAE | immune-related adverse event |
| iRECIST | Response Evaluation Criteria in Solid Tumors |
| | 1.1 for immune-based therapeutics |
| iSD | iRECIST stable disease |
| IT | Intratumoral |
| ITT | immunoglobulin tail tyrosine |
| ITIM | immunoreceptor tyrosine-based inhibitory motif |
| iUPD | iRECIST unconfirmed progressive disease |
| IV | Intravenous |
| IVD | in vitro diagnostic |
| kg | Kilogram |
| LVEF | left ventricular ejection fraction |
| mAb | monoclonal antibody |
| MedDRA | Medical Dictionary for Regulatory Activities |
| Mg | Milligram |
| nL | Milliliter |
| Mm | Millimeter |
| mm³ | cubic millimeter, 0.001 mL |
| MPK | milligram per kilogram |
| MRI | magnetic resonance imaging |
| MSD | Merck Sharp and Dohme Corp., a subsidiary of |
| | Merck & Co., Inc. |
| MSI | microsatellite instability |
| mTPI | modified Toxicity Probability Interval |
| MUGA | multiple-gated acquisition |
| N | number of subjects in a treatment group |
| NCI | National Cancer Institute |
| NE | not estimable |
| NK | Natural killer (cells) |
| NK T | Natural killer T (cells) |
| NSCLC | non-small cell lung cancer |
| OR | overall response |
| ORR | objective response rate |
| OS | overall survival |
| PBPK | physiologically-based PK |
| PD-1 | programmed cell-death 1 |
| PDGFR | platelet-derived growth factor receptor |
| PD-L1 | programmed cell-death ligand 1 |
| PD-L2 | programmed cell-death ligand 2 |
| PE | Physical examination |
| PET | positron emission tomography |
| PFS | Progression-free survival |
| PK | Pharmacokinetic |
| PO | by mouth (orally) |
| PP | Per Protocol |
| pPR | pathological partial response |
| PR | partial response |
| PRO | patient-reported outcome |
| pRR | pathological response rate |
| PT | prothrombin time |
| PTT | partial thromboplastin time |
| Q2W | every 2 weeks |
| Q3W | every 3 weeks |
| Q6W | every 6 weeks |
| Q9W | every 9 weeks |
| Q12W | every 12 weeks |
| Q24W | every 24 weeks |
| QD | once daily/one dose per day |
| Q5D | dose every 5 days |
| Q7D | dose every 7 days |
| Q10D | dose every 10 days |
| QoL | quality of life |
| QTc | The QTc interval is the length of time it take the |
| | electrical system in the heart to repolarize, |
| | adjusted for heart rate. |
| RCC | renal cell carcinoma |
| RECIST | Response Evaluation Criteria in Solid Tumors |
| RECIST 1.1 | Response Evaluation Criteria in Solid Tumors 1.1 |
| RENCA | Renal cell carcinoma |
| RNA | ribonucleic acid |
| RP2D | recommended Phase 2 dose |
| RR | Respiratory rate |
| SAE | serious adverse event |
| SD | stable disease |
| SEM | standard error of the mean |
| SoA | Schedule of Activities |
| T3 | Triiodothyronine |
| T/C | median tumor volume of the treated animal/Median |
| | tumor volume of the control animal |
| TIGIT | T cell immunoreceptor with Ig and ITIM domains |
| TSH | thyroid-stimulating hormone |
| μg, ug, UG | Microgram |
| US | United States |
| V_{H} | immunoglobulin heavy chain variable region |
| V_{K} | immunoglobulin kappa light chain variable region |
| V_{L} | immunoglobulin light chain variable region |
| WOCBP | woman/women of childbearing potential |

### PD-1 Antagonists

Provided herein are PD-1 antagonists or anti-human PD-1 monoclonal antibodies that can be used in any of the methods, compositions, kits, and uses disclosed herein, including any chemical compound or biological molecule that blocks binding of PD-L1 to PD-1 and preferably also blocks binding of PD-L2 to PD-1.

Any monoclonal antibodies that bind to a PD-1 polypeptide, a PD-1 polypeptide fragment, a PD-1 peptide, or a PD-1 epitope and block the interaction between PD-1 and its ligand PD-L1 or PD-L2 can be used. In some embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1. In other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L2. In yet other embodiments, the anti-human PD-1 monoclonal antibody binds to a PD-1 polypeptide, a PD-1 polypeptide fragment, a PD-1 peptide, or a PD-1 epitope and blocks the interaction between PD-1 and PD-L1 and the interaction between PD-1 and PD-L2.

Any monoclonal antibodies that bind to a PD-L1 polypeptide, a PD-L1 polypeptide fragment, a PD-L1 peptide, or a PD-L1 epitope and block the interaction between PD-L1 and PD-1 can also be used.

In certain embodiments, the anti-human PD-1 monoclonal antibody is selected from the group consisting of pembrolizumab, nivolumab, cemiplimab, pidilizumab (U.S. Pat. No. 7,332,582), AMP-514 (MedImmune LLC, Gaithersburg, MD), PDR001 (U.S. Pat. No. 9,683,048), BGB-A317 (U.S. Pat. No. 8,735,553), and MGA012 (MacroGenics, Rockville, MD). In one embodiment, the anti-human PD-1 monoclonal antibody is pembrolizumab. In one embodiment, the anti-human PD-1 monoclonal antibody is pembrolizumab. In another embodiment, the anti-human PD-1 monoclonal antibody is nivolumab. In another embodiment, the anti-human PD-1 monoclonal antibody is cemiplimab. In yet another embodiment, the anti-human PD-1 monoclonal antibody is pidilizumab. In one embodiment, the anti-human PD-1 monoclonal antibody is AMP-514. In another embodiment, the anti-human PD-1 monoclonal antibody is PDR001. In yet another embodiment, the anti-human PD-1 monoclonal antibody is BGB-A317. In still another embodiment, the anti-human PD-1 monoclonal antibody is MGA012.

In some embodiments, an anti-human PD-1 antibody or antigen binding fragment thereof for use in the methods, kits, uses and co-formulations of the invention that comprises three light chain CDRs of CDRL1, CDRL2 and CDRL3 and/or three heavy chain CDRs of CDRH1, CDRH2 and CDRH3.

In one embodiment of the invention, CDRL1 has the amino acid sequence as set forth in SEQ ID NO:1 or a variant of the amino acid sequence as set forth in SEQ ID NO:1, CDRL2 has the amino acid sequence as set forth in SEQ ID NO:2 or a variant of the amino acid sequence as set forth in SEQ ID NO:2, and CDRL3 has the amino acid sequence as set forth in SEQ ID NO:3 or a variant of the amino acid sequence as set forth in SEQ ID NO:3.

In one embodiment, CDRH1 has the amino acid sequence as set forth in SEQ ID NO:6 or a variant of the amino acid sequence as set forth in SEQ ID NO:6, CDRH2 has the amino acid sequence as set forth in SEQ ID NO: 7 or a variant of the amino acid sequence as set forth in SEQ ID NO:7, and CDRH3 has the amino acid sequence as set forth in SEQ ID NO:8 or a variant of the amino acid sequence as set forth in SEQ ID NO:8.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In an alternative embodiment of the invention, CDRL1 has the amino acid sequence as set forth in SEQ ID NO:11 or a variant of the amino acid sequence as set forth in SEQ ID NO:11, CDRL2 has the amino acid sequence as set forth in SEQ ID NO:12 or a variant of the amino acid sequence as set forth in SEQ ID NO:12, and CDRL3 has the amino acid sequence as set forth in SEQ ID NO:13 or a variant of the amino acid sequence as set forth in SEQ ID NO:13.

In one embodiment, CDRH1 has the amino acid sequence as set forth in SEQ ID NO:16 or a variant of the amino acid sequence as set forth in SEQ ID NO:16, CDRH2 has the amino acid sequence as set forth in SEQ ID NO:17 or a variant of the amino acid sequence as set forth in SEQ ID NO:17, and CDRH3 has the amino acid sequence as set forth in SEQ ID NO:18 or a variant of the amino acid sequence as set forth in SEQ ID NO:18.

In one embodiment, the three light chain CDRs have the amino acid sequences as set forth in SEQ ID NO:1, SEQ ID NO:2, and SEQ ID NO:3 and the three heavy chain CDRs have the amino acid sequences as set forth in SEQ ID NO:6, SEQ ID NO:7 and SEQ ID NO:8.

In an alternative embodiment, the three light chain CDRs have the amino acid sequences as set forth in SEQ ID NO:11, SEQ ID NO:12, and SEQ ID NO:13 and the three heavy chain CDRs have the amino acid sequences as set forth in SEQ ID NO:16, SEQ ID NO:17 and SEQ ID NO:18.

In a further embodiment of the invention, CDRL1 has the amino acid sequence as set forth in SEQ ID NO:21 or a variant of the amino acid sequence as set forth in SEQ ID NO:21, CDRL2 has the amino acid sequence as set forth in SEQ ID NO:22 or a variant of the amino acid sequence as set forth in SEQ ID NO:22, and CDRL3 has the amino acid sequence as set forth in SEQ ID NO:23 or a variant of the amino acid sequence as set forth in SEQ ID NO:23.

In yet another embodiment, CDRH1 has the amino acid sequence as set forth in SEQ ID NO:24 or a variant of the amino acid sequence as set forth in SEQ ID NO:24, CDRH2 has the amino acid sequence as set forth in SEQ ID NO: 25 or a variant of the amino acid sequence as set forth in SEQ ID NO:25, and CDRH3 has the amino acid sequence as set forth in SEQ ID NO:26 or a variant of the amino acid sequence as set forth in SEQ ID NO:26.

In another embodiment, the three light chain CDRs have the amino acid sequences as set forth in SEQ ID NO:21, SEQ ID NO:22, and SEQ ID NO:23 and the three heavy chain CDRs have the amino acid sequences as set forth in SEQ ID NO:24, SEQ ID NO:25 and SEQ ID NO:26.

Some anti-human PD-1 antibody and antigen binding fragments of the invention comprise a light chain variable region and a heavy chain variable region. In some embodiments, the light chain variable region comprises the amino acid sequence as set forth in SEQ ID NO:4 or a variant of the amino acid sequence as set forth in SEQ ID NO:4, and the heavy chain variable region comprises the amino acid sequence as set forth in SEQ ID NO:9 or a variant of the amino acid sequence as set forth in SEQ ID NO:9. In further embodiments, the light chain variable region comprises the amino acid sequence as set forth in SEQ ID NO:14 or a variant of the amino acid sequence as set forth in SEQ ID NO:14, and the heavy chain variable region comprises the amino acid sequence as set forth in SEQ ID NO:19 or a variant of the amino acid sequence as set forth in SEQ ID NO:19. In further embodiments, the heavy chain variable region comprises the amino acid sequence as set forth in SEQ ID NO:27 or a variant of the amino acid sequence as set forth in SEQ ID NO:27 and the light chain variable region comprises the amino acid sequence as set forth in SEQ ID NO:28 or a variant of the amino acid sequence as set forth in SEQ ID NO:28, SEQ ID NO:29 or a variant of the amino acid sequence as set forth in SEQ ID NO:29, or SEQ ID NO:30 or a variant of the amino acid sequence as set forth in SEQ ID NO:30. In such embodiments, a light chain variable region or heavy chain variable region sequence is identical to the reference sequence except having one, two, three, four or five amino acid substitutions. In some embodiments, the substitutions are in the framework region (i.e., outside of the CDRs). In some embodiments, one, two, three, four or five of the amino acid substitutions are conservative substitutions.

In one embodiment of the co-formulations, methods, kits or uses of the invention, the anti-human PD-1 antibody or antigen binding fragment comprises a light chain variable region comprising or consisting of the amino acid sequence as set forth in SEQ ID NO:4 and a heavy chain variable region comprising or consisting of the amino acid sequence as set forth in SEQ ID NO:9. In a further embodiment, the anti-human PD-1 antibody or antigen binding fragment comprises a light chain variable region comprising or consisting of the amino acid sequence as set forth in SEQ ID NO:14 and a heavy chain variable region comprising or consisting of the amino acid sequence as set forth in SEQ ID NO:19. In one embodiment of the formulations of the invention, the anti-human PD-1 antibody or antigen binding fragment comprises a light chain variable region comprising or consisting of the amino acid sequence as set forth in SEQ ID NO:28 and a heavy chain variable region comprising or consisting of the amino acid sequence as set forth in SEQ ID NO:27. In a further embodiment, the anti-human PD-1 antibody or antigen binding fragment comprises a light chain variable region comprising or consisting of the amino acid sequence as set forth in SEQ ID NO:29 and a heavy chain variable region comprising or consisting of the amino acid sequence as set forth in SEQ ID NO:27. In another embodiment, the antibody or antigen binding fragment comprises a light chain variable region comprising or consisting of the amino acid sequence as set forth in SEQ ID NO:30 and a heavy chain variable region comprising or consisting of the amino acid sequence as set forth in SEQ ID NO:27.

In another embodiment, the co-formulations, methods, kits or uses of the invention comprise an anti-human PD-1 antibody or antigen binding protein that has a V_{L} domain and/or a V_{H} domain with at least 95%, 90%, 85%, 80%, 75% or 50% sequence homology to one of the V_{L} domains or V_{H} domains described above, and exhibits specific binding to PD-1. In another embodiment, the anti-human PD-1 antibody or antigen binding protein of the co-formulations of the invention comprises V_{L} and V_{H} domains having up to 1, 2, 3, 4, or 5 or more amino acid substitutions, and exhibits specific binding to PD-1.

In any of the embodiments above, the PD-1 antagonist may be a full-length anti-PD-1 antibody or an antigen binding fragment thereof that specifically binds human PD-1. In certain embodiments, the PD-1 antagonist is a full-length anti-PD-1 antibody selected from any class of immunoglobulins, including IgM, IgG, IgD, IgA, and IgE. Preferably, the antibody is an IgG antibody. Any isotype of IgG can be used, including IgG₁, IgG₂, IgG₃, and IgG₄. Different constant domains may be appended to the V_{L} and V_{H} regions provided herein. For example, if a particular intended use of an antibody (or fragment) of the present invention were to call for altered effector functions, a heavy chain constant domain other than IgG1 may be used. Although IgG1 antibodies provide for long half-life and for effector functions, such as complement activation and antibody-dependent cellular cytotoxicity, such activities may not be desirable for all uses of the antibody. In such instances an IgG4 constant domain, for example, may be used.

In embodiments of the invention, the PD-1 antagonist is an anti-PD-1 antibody comprising a light chain comprising or consisting of a sequence of amino acid residues as set forth in SEQ ID NO:5 and a heavy chain comprising or consisting of a sequence of amino acid residues as set forth in SEQ ID NO:10. In alternative embodiments, the PD-1 antagonist is an anti-PD-1 antibody comprising a light chain comprising or consisting of a sequence of amino acid residues as set forth in SEQ ID NO:15 and a heavy chain comprising or consisting of a sequence of amino acid residues as set forth in SEQ ID NO:20. In further embodiments, the PD-1 antagonist is an anti-PD-1 antibody comprising a light chain comprising or consisting of a sequence of amino acid residues as set forth in SEQ ID NO:32 and a heavy chain comprising or consisting of a sequence of amino acid residues as set forth in SEQ ID NO:31. In additional embodiments, the PD-1 antagonist is an anti-PD-1 antibody comprising a light chain comprising or consisting of a sequence of amino acid residues as set forth in SEQ ID NO:33 and a heavy chain comprising or consisting of a sequence of amino acid residues as set forth in SEQ ID NO:31. In yet additional embodiments, the PD-1 antagonist is an anti-PD-1 antibody comprising a light chain comprising or consisting of a sequence of amino acid residues as set forth in SEQ ID NO:34 and a heavy chain comprising or consisting of a sequence of amino acid residues as set forth in SEQ ID NO:31. In some co-formulations of the invention, the PD-1 antagonist is pembrolizumab or a pembrolizumab biosimilar. In some co-formulations of the invention, the PD-1 antagonist is nivolumab or a nivolumab biosimilar.

Ordinarily, amino acid sequence variants of the anti-PD-1 antibodies and antigen binding fragments of the invention, and the anti-TIGIT antibodies and antigen binding fragments will have an amino acid sequence having at least 75% amino acid sequence identity with the amino acid sequence of a reference antibody or antigen binding fragment (e.g. heavy chain, light chain, V_{H}, V_{L}, or humanized sequence), more preferably at least 80%, more preferably at least 85%, more preferably at least 90%, and most preferably at least 95, 98, or 99%. Identity or homology with respect to a sequence is defined herein as the percentage of amino acid residues in the candidate sequence that are identical with the reference antibody or antigen binding fragment residues, after aligning the sequences and introducing gaps, if necessary, to achieve the maximum percent sequence identity, and not considering any conservative substitutions as part of the sequence identity. None of N-terminal, C-terminal, or internal extensions, deletions, or insertions into the antibody sequence shall be construed as affecting sequence identity or homology.

Sequence identity refers to the degree to which the amino acids of two polypeptides are the same at equivalent positions when the two sequences are optimally aligned. Sequence identity can be determined using a BLAST algorithm wherein the parameters of the algorithm are selected to give the largest match between the respective sequences over the entire length of the respective reference sequences. The following references relate to BLAST algorithms often used for sequence analysis: BLAST ALGORITHMS: Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, DC; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, DC; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

Likewise, either class of light chain can be used in the compositions and methods herein. Specifically, kappa, lambda, or variants thereof are useful in the present compositions and methods.

TABLE 4
Exemplary PD-1 Antibody Sequences

| | | |
|---|---|---|
| Antibody | | SEQ ID |
| Feature | Amino Acid Sequence | NO. |

Pembrolizumab Light Chain

| | | |
|---|---|---|
| CDR1 | RASKGVSTSGYSYLH | 1 |
| CDR2 | LASYLES | 2 |
| CDR3 | QHSRDLPLT | 3 |
| Variable | EIVLTQSPATLSLSPGERATLSCRASKGVSTSGYSYLHWYQQKPGQAP | 4 |
| Region | RLLIYLASYLESGVPARFSGSGSGTDFTLTISSLEPEDFAVYYCQHSR | |
| | DLPLTFGGGTKVEIK | |
| Light Chain | EIVLTQSPATLSLSPGERATLSCRASKGVSTSGYSYLHWYQQKPGQAP | 5 |
| | RLLIYLASYLESGVPARFSGSGSGTDFTLTISSLEPEDFAVYYCQHSR | |
| | DLPLTFGGGTKVEIKRTVAAPSVFIFPPSDEQLKSGTASVVCLLNNFY | |
| | PREAKVQWKVDNALQSGNSQESVTEQDSKDSTYSLSSTLTLSKADYEK | |
| | HKVYACEVTHQGLSSPVTKSFNRGEC | |

Pembrolizumab Heavy Chain

| | | |
|---|---|---|
| CDR1 | NYYMY | 6 |
| CDR2 | GINPSNGGTNFNEKFKN | 7 |
| CDR3 | RDYRFDMGFDY | 8 |
| Variable | QVQLVQSGVEVKKPGASVKVSCKASGYTFTNYYMYWVRQAPGQGL | 9 |
| Region | EWMGGINPSNGGTNFNEKFKNRVTLTTDSSTTTAYMELKSLQFDD | |
| | TAVYYCARRDYRFDMGFDYWGQGTTVTVSS | |
| Heavy Chain | QVQLVQSGVEVKKPGASVKVSCKASGYTFTNYYMYWVRQAPGQGL | 10 |
| | EWMGGINPSNGGTNFNEKFKNRVTLTTDSSTTTAYMELKSLQFDD | |
| | TAVYYCARRDYRFDMGFDYWGQGTTVTVSSASTKGPSVFPLAPCS | |
| | RSTSESTAALGCLVKDYFPEPVTVSWNSGALTSGVHTFPAVLQSS | |
| | GLYSLSSVVTVPSSSLGTKTYTCNVDHKPSNTKVDKRVESKYGPP | |
| | CPPCPAPEFLGGPSVFLFPPKPKDTLMISRTPEVTCVVVDVSQED | |
| | PEVQFNWYVDGVEVHNAKTKPREEQFNSTYRVVSVLTVLHQDWLN | |
| | GKEYKCKVSNKGLPSSIEKTISKAKGQPREPQVYTLPPSQEEMTK | |
| | NQVSLTCLVKGFYPSDIAVEWESNGQPENNYKTTPPVLDSDGSFF | |
| | LYSRLTVDKSRWQEGNVFSCSVMHEALHNHYTQKSLSLSLGK | |

Nivolumab Light Chain

| | | |
|---|---|---|
| CDR1 | RASQSVSSYLA | 11 |
| CDR2 | DASNRAT | 12 |
| CDR3 | QQSSNWPRT | 13 |
| Variable | EIVLTQSPATLSLSPGERATLSCRASQSVSSYLAWYQQKPGQAPRL | 14 |
| Region | LIYDASNRATGIPARFSGSGSGTDFTLTISSLEPEDFAVYYCQQSS | |
| | NWPRTFGQGTKVEIK | |
| Light Chain | EIVLTQSPATLSLSPGERATLSCRASQSVSSYLAWYQQKPGQAPRL | 15 |
| | LIYDASNRATGIPARFSGSGSGTDFTLTISSLEPEDFAVYYCQQSS | |
| | NWPRTFGQGTKVEIKRTVAAPSVFIFPPSDEQLKSGTASVVCLLNN | |
| | FYPREAKVQWKVDNALQSGNSQESVTEQDSKDSTYSLSSTLTLSKA | |
| | DYEKHKVYACEVTHQGLSSPVTKSFNRGEC | |

Nivolumab Heavy Chain

| | | |
|---|---|---|
| CDR1 | NSGMH | 16 |
| CDR2 | VIWYDGSKRYYADSVKG | 17 |
| CDR3 | NDDY | 18 |
| Variable | QVQLVESGGGVVQPGRSLRLDCKASGITFSNSGMHWVRQAPGKGLE | 19 |
| Region | WVAVIWYDGSKRYYADSVKGRFTISRDNSKNTLFLQMNSLRAEDTA | |
| | VYYCATNDDYWGQGTLVTVSS | |
| Heavy Chain | QVQLVESGGGVVQPGRSLRLDCKASGITFSNSGMHWVRQAPGKGLE | 20 |
| | WVAVIWYDGSKRYYADSVKGRFTISRDNSKNTLFLQMNSLRAEDTA | |
| | VYYCATNDDYWGQGTLVTVSSASTKGPSVFPLAPCSRSTSESTAAL | |
| | GCLVKDYFPEPVTVSWNSGALTSGVHTFPAVLQSSGLYSLSSVVTV | |
| | PSSSLGTKTYTCNVDHKPSNTKVDKRVESKYGPPCPPCPAPEFLGG | |
| | PSVFLFPPKPKDTLMISRTPEVTCVVVDVSQEDPEVQFNWYVDGVE | |
| | VHNAKTKPREEQFNSTYRVVSVLTVLHQDWLNGKEYKCKVSNKGLP | |
| | SSIEKTISKAKGQPREPQVYTLPPSQEEMTKNQVSLTCLVKGFYPS | |
| | DIAVEWESNGQPENNYKTTPPVLDSDGSFFLYSRLTVDKSRWQEGN | |
| | VFSCSVMHEALHNHYTQKSLSLSLGK | |

TABLE 5
Additional PD-1 Antibodies and Antigen Binding
Fragments Useful in the Co-Formulations, Compositions,
Methods, Kits and Uses of the Invention.
A. Antibodies and antigen binding fragments comprising light
and heavy chain CDRs of hPD-1.08A in WO2008/156712

| | |
|---|---|
| CDRL1 | SEQ ID NO: 21 |
| CDRL2 | SEQ ID NO: 22 |
| CDRL3 | SEQ ID NO: 23 |
| CDRH1 | SEQ ID NO: 24 |
| CDRH2 | SEQ ID NO: 25 |
| CDRH3 | SEQ ID NO: 26 |

C. Antibodies and antigen binding fragments comprising the mature
h109A heavy chain variable region and one of the mature
K09A light chain variable regions in WO 2008/156712

| | |
|---|---|
| Heavy chain VR | SEQ ID NO: 27 |
| Light chain VR | SEQ ID NO: 28 or SEQ ID NO: 29 or SEQ ID NO: 30 |

D. Antibodies and antigen binding fragments comprising the mature
409 heavy chain and one of the mature K09A light chains
in WO 2008/156712

| | |
|---|---|
| Heavy chain | SEQ ID NO: 31 |
| Light chain | SEQ ID NO: 32 or SEQ ID NO: 33 or SEQ ID NO: 34 |

Provided herein are anti-human TIGIT monoclonal antibodies or antigen binding fragments thereof that can be used in the methods, pharmaceutical compositions, kits, and uses disclosed herein. Any monoclonal antibodies that bind to a TIGIT polypeptide, a TIGIT polypeptide fragment, a TIGIT peptide, or a TIGIT epitope and block the interaction between TIGIT and its ligand CD155 and/or CD112 can be used. In some embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155. In other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD112. In yet other embodiments, the anti-human TIGIT monoclonal antibody binds to a TIGIT polypeptide, a TIGIT polypeptide fragment, a TIGIT peptide, or a TIGIT epitope and blocks the interaction between TIGIT and CD155 and the interaction between TIGIT and CD112. In some embodiments the human constant region is selected from the group consisting of IgG1, IgG2, IgG3 and IgG4 constant regions, and in preferred embodiments, the human constant region is an IgG1 or IgG4 constant region.

Exemplary anti-TIGIT antibody sequences are set forth below in Tables 6 and 7.

TABLE 6
Exemplary anti-TIGIT antibodies

| | SEQ | |
|---|---|---|
| | ID | |
| Description | NO: | SEQUENCE |
| 14A6 H-CDR1 | 35 | SDYWG |
| 14A6 H-CDR2 | 36 | FITYSGSTSYNPSLKS |
| 14A6 H-CDR3 | 37 | MPSFITLASLSTWEGYFDF |
| 14A6 L-CDR1 | 38 | KASQSIHKNLA |
| 14A6 L-CDR2 | 39 | YANSLQT |
| 14A6 L-CDR3 | 40 | QQYYSGWT |
| 14A6 PARENTALVH | 41 | EVQLQESGPGLVKPSQSLSLTCSVTGSSIASDYWGWIR |
| | | KFPGNKMEWMGFITYSGSTSYNPSLKSRISITRDTSKN |
| | | QFFLQLHSVTTDDTATYSCARMPSFITLASLSTWEGYF |
| | | DFWGPGTMVTVSS |
| 14A6 PARENTALVL | 42 | DIQMTQSPSLLSASVGDRVTLNCKASQSIHKNLAWYQ |
| | | QKLGEAPKFLIYYANSLQTGIPSRFSGSGSGTDFTLTIS |
| | | GLQPEDVATYFCQQYYSGWTFGGGTKVELK |
| Hu14A6VH.1 | 43 | EVQLQESGPGLVKPSETLSLTCTVSGGSISSDYWGWIR |
| | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| Hu14A6VH.1a | 44 | EVQLQESGPGLVKPSETLSLTCTVSGGSISSDYWGWIR |
| | | QPPGKGLEWIGFITYSGSTSYNPSLKSRVTISRDTSKN |
| | | QFSLKLSSVTAADTAVYYCARMPSFITLASLSTWEGYF |
| | | DFWGQGTMVTVSS |
| Hu14A6VH.1b | 45 | EVQLQESGPGLVKPSETLSLTCTVSGGSISSDYWGWIR |
| | | QPPGKGLEWIGFITYSGSTSYNPSLKSRITISRDTSKN |
| | | QFSLKLSSVTAADTAVYYCARMPSFITLASLSTWEGYF |
| | | DFWGQGTMVTVSS |
| Hu14A6VH.1c | 46 | EVQLQESGPGLVKPSETLSLTCTVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGY |
| | | FDFWGQGTMVTVSS |
| Hu14A6VH.1d | 47 | EVQLQESGPGLVKPSETLSLTCTVSGGSISSDYWGWIR |
| | | QPPGKGLEWIGFITYSGSTSYNPSLKSRVTISRDTSKN |
| | | QFSLKLHSVTAADTAVYYCARMPSFITLASLSTWEGYF |
| | | DFWGQGTMVTVSS |
| Hu14A6VH.1e | 48 | EVQLQESGPGLVKPSETLSLTCTVSGGSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.If | 49 | EVQLQESGPGLVKPSETLSLTCTVSGSSISSDYWGWIR |
| | | QPPGKGLEWIGFITYSGSTSYNPSLKSRITISRDTSKN |
| | | QFSLKLHSVTAADTAVYYCARMPSFITLASLSTWEGYF |
| | | DFWGQGTMVTVSS |
| Hu14A6VH.1g | 50 | EVQLQESGPGLVKPSETLSLTCTVSGSSISSDYWGWIR |
| | | QPPGKGLEWMGFITYSGSTSYNPSLKSRITISVDTSKN |
| | | QFSLKLHSVTAADTAVYYCARMPSFITLASLSTWEGY |
| | | FDFWGQGTMVTVSS |
| Hu14A6VH.2 | 51 | EVQLQESGPGLVKPSETLSLTCAVSGYSISSDYWGWIR |
| | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| Hu14A6VH.2a | 52 | EVQLQESGPGLVKPSETLSLTCAVSGYSISSDYWGWIR |
| | | QPPGKGLEWIGFITYSGSTSYNPSLKSRVTISRDTSKNQ |
| | | FSLKLSSVTAADTAVYYCARMPSFITLASLSTWEGYF |
| | | DFWGQGTMVTVSS |
| Hu14A6VH.2b | 53 | EVQLQESGPGLVKPSETLSLTCAVSGYSISSDYWGWIR |
| | | QPPGKGLEWIGFITYSGSTSYNPSLKSRITISRDTSKNQ |
| | | FSLKLSSVTAADTAVYYCARMPSFITLASLSTWEGYF |
| | | DFWGQGTMVTVSS |
| Hu14A6VH.2c | 54 | EVQLQESGPGLVKPSETLSLTCAVSGSSISSDYWGWIR |
| | | QPPGKGLEWMGFITYSGSTSYNPSLKSRITISRDTSKN |
| | | QFSLKLSSVTAADTAVYYCARMPSFITLASLSTWEGY |
| | | FDFWGQGTMVTVSS |
| Hu14A6VH.2d | 55 | EVQLQESGPGLVKPSETLSLTCAVSGYSISSDYWGWIR |
| | | QPPGKGLEWIGFITYSGSTSYNPSLKSRVTISRDTSKNQ |
| | | FSLKLHSVTAADTAVYYCARMPSFITLASLSTWEGYF |
| | | DFWGQGTMVTVSS |
| Hu14A6VH.2e | 56 | EVQLQESGPGLVKPSETLSLTCAVSGYSISSDYWGWIR |
| | | QPPGKGLEWIGFITYSGSTSYNPSLKSRITISRDTSKNQ |
| | | FSLKLHSVTAADTAVYYCARMPSFITLASLSTWEGYF |
| | | DFWGQGTMVTVSS |
| Hu14A6VH.2f | 57 | EVQLQESGPGLVKPSETLSLTCAVSGSSISSDYWGWIR |
| | | QPPGKGLEWIGFITYSGSTSYNPSLKSRITISRDTSKNQ |
| | | FSLKLHSVTAADTAVYYCARMPSFITLASLSTWEGYF |
| | | DFWGQGTMVTVSS |
| Hu14A6VH.2e | 58 | EVQLQESGPGLVKPSETLSLTCAVSGSSISSDYWGWIR |
| | | QPPGKGLEWMGFITYSGSTSYNPSLKSRITISRDTSKN |
| | | QFSLKLHSVTAADTAVYYCARMPSFITLASLSTWEGY |
| | | FDFWGQGTMVTVSS |
| Hu14A6Vk.1 | 59 | DIQMTQSPSSLSASVGDRVTITCKASQSIHKNLAWYQ |
| | | QKPGKAPKLLIYYANSLQTGVPSRFSGSGSGTDFTLTI |
| | | SSLQPEDFATYYCQQYYSGWTFGGGTKVEIK |
| Hu14A6Vk.1a | 60 | DIQMTQSPSSLSASVGDRVTITCKASQSIHKNLAWYQ |
| | | QKPGKAPKFLIYYANSLQTGVPSRFSGSGSGTDFTLTI |
| | | SSLQPEDFATYYCQQYYSGWTFGGGTKVEIK |
| Hu14A6Vk.1b | 61 | DIQMTQSPSSLSASVGDRVTITCKASQSIHKNLAWYQ |
| | | QKPGKAPKFLIYYANSLQTGIPSRFSGSGSGTDFTLTIS |
| | | SLQPEDFATYYCQQYYSGWTFGGGTKVEIK |
| Hu14A6Vk.2 | 62 | DIQMTQSPSSLSASVGDRVTITCKASQSIHKNLAWYQ |
| | | QKPGKVPKLLIYYANSLQTGVPSRFSGSGSGTDFTLTI |
| | | SSLQPEDVATYYCQQYYSGWTFGGGTKVEIK |
| Hu14A6Vk.2a | 63 | DIQMTQSPSSLSASVGDRVTITCKASQSIHKNLAWYQ |
| | | QKPGKVPKFLIYYANSLQTGVPSRFSGSGSGTDFTLTI |
| | | SSLQPEDVATYYCQQYYSGWTFGGGTKVEIK |
| Hu14A6Vk.2b | 64 | DIQMTQSPSSLSASVGDRVTITCKASQSIHKNLAWYQ |
| | | QKPGKVPKFLIYYANSLQTGIPSRFSGSGSGTDFTLTIS |
| | | SLQPEDVATYYCQQYYSGWTFGGGTKVEIK |
| 16AHA_tigit_14a6_humanized_VH1 | 65 | EVQLQESGPGLVKPSETLSLTCTVSGSSIASDYWGWIR |
| LB155.14A6.G2.A8_VH1 | | QPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSSAS |
| 18AHA_tigit_14a6_humanized_VH2 | 66 | EVQLQESGPGLVKPSETLSLTCTVSGSSIASDYWGWIR |
| LB155.14A6.G2.A8_VH2 | | QPPGKGLEWIGFITYSGSTSYNPSLKSRVTISRDTSKNQ |
| | | FSLKLSSVTAADTAVYYCARMPSFITLASLSTWEGYF |
| | | DFWGQGTMVTVSS |
| 20AHA_tigit_14a6_humanized_VH3 | 67 | EVQLQESGPGLVKPSETLSLTCTVSGSSIASDYWGWIR |
| LB155.14A6.G2.A8_VH3 | | KPPGKGLEWIGFITYSGSTSYNPSLKSRVTISRDTSKNQ |
| | | FSLKLSSVTAADTAVYYCARMPSFITLASLSTWEGYF |
| | | DFWGQGTMVTVSS |
| 21AHA_tigit_14a6_humanized_VH4 | 68 | EVQLQESGPGLVKPSETLSLTCTVSGSSIASDYWGWIR |
| LB155.14A6.G2.A8_VH4 | | QPPGKKLEWIGFITYSGSTSYNPSLKSRVTISRDTSKNQ |
| | | FSLKLSSVTAADTAVYYCARMPSFITLASLSTWEGYF |
| | | DFWGQGTMVTVSS |
| 19AHA_tigit_14a6_humanized_VH5 | 69 | EVQLQESGPGLVKPSETLSLTCTVSGSSIASDYWGWIR |
| LB155.14A6.G2.A8_VH5 | | QPPGKGMEWIGFITYSGSTSYNPSLKSRVTISRDTSKN |
| | | QFSLKLSSVTAADTAVYYCARMPSFITLASLSTWEGY |
| | | FDFWGQGTMVTVSS |
| 22AHA_tigit_14a6_humanized_VH6 | 70 | EVQLQESGPGLVKPSETLSLTCTVSGSSIASDYWGWIR |
| LB155.14A6.G2.A8_VH6 | | KPPGKKMEWIGFITYSGSTSYNPSLKSRVTISRDTSKN |
| | | QFSLKLSSVTAADTAVYYCARMPSFITLASLSTWEGY |
| | | FDFWGQGTMVTVSS |
| 23AHA_tigit_14a6_humanized_VH7 | 71 | EVQLQESGPGLVKPSETLSLTCTVSGSSIASDYWGWIR |
| LB155.14A6.G2.A8_VH7 | | QFPGKGLEWIGFITYSGSTSYNPSLKSRVTISRDTSKNQ |
| | | FSLKLSSVTADDTAVYYCARMPSFITLASLSTWEGYF |
| | | DFWGQGTMVTVSS |
| 24AHA_tigit_14a6_humanized_VH8 | 72 | EVQLQESGPGLVKPSETLSLTCTVSGSSIASDYWGWIR |
| LB155.14A6.G2.A8_VH8 | | KPPGKKMEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 25AHA_tigit_14a6_humanized_VH9 | 73 | EVQLQESGPGLVKPSETLSLTCSVTGSSIASDYWGWIR |
| LB155.14A6.G2.A8_VH9 | | QPPGKGLEWIGFITYSGSTSYNPSLKSRVTISRDTSKNQ |
| | | FSLKLSSVTAADTAVYYCARMPSFITLASLSTWEGYF |
| | | DFWGQGTMVTVSS |
| 26AHA_tigit_14a6_humanized_VH10 | 74 | EVQLQQSGAGLLKPSETLSLTCSVTGSSIASDYWGWI |
| LB155.14A6.G2.A8_VH10 | | RQPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 27AHA_tigit_14a6_humanized_VH11 | 75 | EVQLQESGPGLVKPPGTLSLTCSVTGSSIASDYWGWV |
| LB155.14A6.G2.A8_VH11 | | RQPPGKGLEWIGFITYSGSTSYNPSLKSRVTISVDTSKN |
| | | QFSLKLSSVTAADTAVYYCARMPSFITLASLSTWEGY |
| | | FDFWGQGTMVTVSS |
| 09AHA_tigit_14a6_humanized_VL1 | 76 | DIQMTQSPSSLSASVGDRVTITCKASQSIHKNLAWYQ |
| LB155.14A6.G2.A8_VL1 | | QKPGKAPKLLIYYANSLQTGVPSRFSGSGSGTDFTLTI |
| | | SSLQPEDFATYYCQQYYSGWTFGGGTKVEIK |
| 11AHA_tigit_14a6_humanized_VL2 | 77 | DIQMTQSPSSLSASVGDRVTITCKASQSIHKNLAWYQ |
| LB155.14A6.G2.A8_VL2 | | QKPGKAPKFLIYYANSLQTGVPSRFSGSGSGTDFTLTI |
| | | SSLQPEDFATYYCQQYYSGWTFGGGTKVEIK |
| 12AHA_tigit_14a6_humanized_VL3 | 78 | DIQMTQSPSSLSASVGDRVTITCKASQSIHKNLAWYQ |
| LB155.14A6.G2.A8_VL3 | | QKPGKAPKLLIYYANSLQTGVPSRFSGSGSGTDFTLTI |
| | | SSLQPEDFATYFCQQYYSGWTFGGGTKVEIK |
| 13AHA_tigit_14a6_humanized_VL4 | 79 | DIQMTQSPSSLSASVGDRVTITCKASQSIHKNLAWYQ |
| LB155.14A6.G2.A8_VLA | | QKPGKAPKFLIYYANSLQTGVPSRFSGSGSGTDFTLTI |
| | | SSLQPEDFATYFCQQYYSGWTFGGGTKVEIK |
| 15AHA_tigit_14a6_humanized_VL5 | 80 | DIQMTQSPSSLSASVGDRVTITCKASQSIHKNLAWYQ |
| LB155.14A6.G2.A8_VL5 | | QKPGKAPKLLIYYANSLQTGIPSRFSGSGSGTDFTLTIS |
| | | SLQPEDFATYYCQQYYSGWTFGGGTKVEIK |
| 28H5 H-CDR1 | 81 | GYSITSDYAWN |
| 28H5 H-CDR2 | 82 | YISNSGSASYNPSLKS |
| 28H5 H-CDR3 | 83 | LIYYDYGGAMNF |
| 28H5 L-CDR1 | 84 | KASQGVSTTVA |
| 28H5 L-CDR2 | 85 | SASYRYT |
| 28H5 L-CDR3 | 86 | QHYYSTPWT |
| 28H5 PARENTALVH | 87 | DVQLQESGPGLVKPSQSLSLTCTVTGYSITSDYAWNW |
| | | IRQFPGNKLEWMGYISNSGSASYNPSLKSRISITRDTSK |
| | | NQFFLQLNSVTTEDTATYYCATLIYYDYGGAMNFWG |
| | | QGTSVTVSS |
| 28H5 PARENTALVL | 88 | DIVMTQSHKFMSTSVGDRVSITCKASQGVSTTVAWY |
| | | QQKPGQSPKLLIYSASYRYTGVPDRFTGSGSGTDFTFT |
| | | ISSVQSEDLAVYYCQHYYSTPWTFGGGTKLEIK |
| 14H6 L-CDR2 variant | 89 | YASNLQT |
| 14H6 L-CDR2 variant | 90 | YASSLQT |
| 14H6 L-CDR2 variant | 91 | YASTLQT |
| 14H6 L-CDR2 variant | 92 | YATTLQT |
| 14H6 L-CDR2 variant | 93 | YASYLQT |
| 14H6 L-CDR2 variant | 94 | YANQLQT |
| 14H6 L-CDR2 variant | 95 | YAGSLQT |
| 14H6 L-CDR2 variant | 96 | YASQLQT |
| 14H6 L-CDR2 variant | 97 | YADSLQT |
| 14H6 L-CDR3 variant | 98 | QQYYSGFT |
| 14H6 L-CDR3 variant | 99 | QQYYSGYT |
| 14H6 L-CDR3 variant | 100 | QQYYSGIT |
| 14H6 L-CDR3 variant | 101 | QQYYSGVT |
| 14H6 L-CDR3 variant | 102 | QQYYSGLT |
| 14H6 H-CDR3 variant | 103 | MPSFITLASLSTFEGYFDF |
| 14H6 H-CDR3 variant | 104 | MPSFITLASLSTYEGYFDF |
| 14H6 H-CDR3 variant | 105 | MPSFITLASLSTIEGYFDF |
| 14H6 H-CDR3 variant | 106 | MPSFITLASLSTVEGYFDF |
| 14H6 H-CDR3 variant | 107 | MPSFITLASLSTLEGYFDF |
| 31C6 H-CDR1 | 108 | SYVMH |
| 31C6 H-CDR2 | 109 | YIDPYNDGAKYNEKFKG |
| 31C6 H-CDR3 | 110 | GGPYGWYFDV |
| 31C6 L-CDR1 | 111 | RASEHIYSYLS |
| 31C6 L-CDR2 | 112 | NAKTLAE |
| 31C6 L-CDR3 | 113 | QHHFGSPLT |
| 31C6 PARENTALVH | 114 | EVQLQQSGPELVKPGSSVKMSCKASGYTFSSYVMHW |
| | | VKQKPGQGLEWIGYIDPYNDGAKYNEKFKGKATLTS |
| | | DKSSSTAYMELSSLTSEDSAVYYCARGGPYGWYFDV |
| | | WGAGTTVTVSS |
| 31C6 PARENTALVL | 115 | DIQMTQSPASLSASVGETVTITCRASEHIYSYLSWYQQ |
| | | KQGKSPQLLVYNAKTLAEGVPSRFSGSGSGTQFSLKI |
| | | NSLQPEDFGTYYCQHHFGSPLTFGAGTTLELK |
| 31C6 H-CDR2 VARIANT (D56R) | 116 | YIDPYNrGAKYNEKFG |
| 31C6 H-CDR2 VARIANT (D56L) | 117 | YIDPYNIGAKYNEKGF |
| 31C6 H-CDR2 VARIANT (D56K) | 118 | YIDPYNKGAKYNEKFG |
| 31C6 H-CDR2 VARIANT (D56F) | 119 | YIDPYNfGAKYNEKFG |
| 31C6 H-CDR2 VARIANT (D56S) | 120 | YIDPYNsGAKYNEKFG |
| 31C6 H-CDR2 VARIANT (D56Y) | 121 | YIDPYNy GAKYNEKFG |
| 31C6 H-CDR2 VARIANT (D56V) | 122 | YIDPYNVGAKYNEKFG |
| 31C6 H-CDR2 VARIANT (G57R) | 123 | YIDPYNDrAKYNEKFKG |
| 31C6 H-CDR2 VARIANT (G57N) | 124 | YIDPYNDnAKYNEKFKG |
| 31C6 H-CDR2 VARIANT (G57Q) | 125 | YIDPYNDqAKYNEKFKG |
| 31C6 H-CDR2 VARIANT (G57E) | 126 | YIDPYNDeAKYNEKFKG |
| 31C6 H-CDR2 VARIANT (G57L) | 127 | YIDPYNDIAKYNEKFKG |
| 31C6 H-CDR2 VARIANT (G57K) | 128 | YIDPYNDKAKYNEKFKG |
| 31C6 H-CDR2 VARIANT (G57S) | 129 | YIDPYNDSAKYNEKFKG |
| 31C6 H-CDR2 VARIANT (G57Y) | 130 | YIDPYNDy AKYNEKFKG |
| 31C6 H-CDR2 VARIANT (G57V) | 131 | YIDPYNDvAKYNEKFKG |
| 31C6 L-CDR2 variant (N50A) | 132 | AAKTLAE |
| 31C6 L-CDR2 variant (N50Y) | 133 | YAKTLAE |
| 31C6 L-CDR2 variant (N50W) | 134 | WAKTLAE |
| 31C6 L-CDR2 variant (N50S) | 135 | SAKTLAE |
| 31C6 L-CDR2 variant (N50T) | 136 | TAKTLAE |
| 31C6 L-CDR2 variant (N50I) | 137 | IAKTLAE |
| 31C6 L-CDR2 variant(N50V) | 138 | VAKTLAE |
| 31C6 L-CDR2 variant (A51N) | 139 | NNKTLAE |
| 31C6 L-CDR2 variant (A51I) | 140 | NIKTLAE |
| 31C6 L-CDR2 variant (A51L) | 141 | NLLTLAE |
| 31C6 L-CDR2 variant (A51T) | 142 | NTKTLAE |
| 31C6 L-CDR2 variant (A51V) | 143 | NVKTLAE |
| 31C6_HUMZ_VH1 | 144 | EVQLVQSGAEVKKPGASVKVSCKASGYTFSSYVMH |
| | | WVRQAPGQRLEWIGYIDPYNDGAKYSQKFQGRVTLT |
| | | RDTSASTAYMELSSLRSEDTAVYYCARGGPYGWYFD |
| | | VWGQGTTVTVSS |
| 31C6_HUMZ_VH2 | 145 | EVQLVQSGAEVKKPGASVKVSCKASGYTFSSYVMH |
| | | WVRQAPGQRLEWIGYIDPYNDGAKYSQKFQGRVTLT |
| | | SDKSASTAYMELSSLRSEDTAVYYCARGGPYGWYFD |
| | | VWGQGTTVTVSS |
| 31C6_HUMZ_VH3 | 146 | EVQLVQSGAEVKKPGASVKVSCKASGYTFSSYVMH |
| | | WVRQAPGQGLEWIGYIDPYNDGAKYAQKFQGRVILT |
| | | RDTSTSTVYMELSSLRSEDTAVYYCARGGPYGWYFD |
| | | VWGQGTTVTVSS |
| 31C6_HUMZ_VH4 | 147 | EVQLVQSGAEVKKPGASVKVSCKASGYTFSSYVMH |
| | | WVRQAPGQGLEWIGYIDPYNDGAKYAQKFQGRVTLT |
| | | SDKSTSTVYMELSSLRSEDTAVYYCARGGPYGWYFD |
| | | VWGQGTTVTVSS |
| 31C6_HUMZ_VH5 | 148 | EVQLVQSGAEVKKPGSSVKVSCKASGYTFSSYVMHW |
| | | VRQAPGQGLEWIGYIDPYNDGAKYAQKFQGRVTLTS |
| | | DKSTSTAYMELSSLRSEDTAVYYCARGGPYGWYFDV |
| | | WGQGTTVTVSS |
| 31C6_HUMZ_VH6 | 149 | EVQLVQSGAEVKKPGASVKVSCKASGYTFSSYVMH |
| | | WVRQAPGQGLEWIGYIDPYNDGAKYAQKFQGRVTLT |
| | | SDKSISTAYMELSRLRSDDTVVYYCARGGPYGWYFD |
| | | VWGQGTTVTVSS |
| 31C6_Humz_L1 | 150 | DIQMTQSPSSLSASVGDRVTITCRASEHIYSYLSWYQQ |
| | | KPGKAPKLLIYNAKTLAEGVPSRFSGSGSGTDFTLTISS |
| | | LQPEDFATYYCQHHFGSPLTFGQGTRLEIK |
| 31C6_Humz_L2 | 151 | DIQMTQSPSSLSASVGDRVTITCRASEHIYSYLSWYQQ |
| | | KPGKAPKLLIYNAKTLAEGVPSRFSGSGSGTQFTLTISS |
| | | LQPEDFATYYCQHHFGSPLTFGQGTRLEIK |
| 31C6_Humz_L3 | 152 | DIQMTQSPSSLSASVGDRVTITCRASEHIYSYLSWYQQ |
| | | KPGKVPKLLIYNAKTLAEGVPSRFSGSGSGTDFTLTISS |
| | | LQPEDVATYYCQHHFGSPLTFGQGTRLEIK |
| 31C6_Humz_L4 | 153 | DIQMTQSPSSLSASVGDRVTITCRASEHIYSYLSWYQQ |
| | | KPGKVPKLLIYNAKTLAEGVPSRFSGSGSGTQFTLTISS |
| | | LQPEDVATYYCQHHFGSPLTFGQGTRLEIK |
| 31C6 H-CDR2 variant | 154 | YIDPYNDGAKYAQKFQG |
| 31C6 H-CDR2 variant | 155 | YIDPYNDGAKYSQKFQG |
| 18G10-VH sequence | 156 | QVQLMESGPGLVQPSQTLSLTCTVSGFPLTSYTVHWV |
| | | RQPPGKGLEWIGAIWSSGSTDYNSALKSRLNINRDSSK |
| | | SQVFLKMNSLQTEDTAIYFCTKSGWAFFDYWGQGVM |
| | | VTVSS |
| 18G10-VL sequence | 157 | DIQMTQSPSLLSASVGDRVTLNCIASQNIYKSLAWYQ |
| | | LKLGEAPKLLIYNANSLQAGIPSRFSGSGSGTDFALTIS |
| | | GLQPEDVATYFCQQYSGGYTFGAGTKLELK |
| 11A11-VH sequence | 158 | EVQLVESGGDLVQPGRSLKISCVASGFTFSDYYMAW |
| | | VRLAPQKGLEWVASISYEGSRTHYGDSVRGRFIISRDN |
| | | PKNILYLQMNSLGSEDTATYFCARHTGTLDWLVYWG |
| | | QGTLVIVSS |
| 11A11-VL sequence | 159 | NIVMAQSPKSMSISAGDRVTMNCKASQNVDNNIAWY |
| | | QQKPGQSPKLLIFYASNRYSGVPDRFTGGGYGTDFTL |
| | | TIKSVQAEDAAFYYCQRIYNFPTFGSGTKLEIK |
| 14A6 H-CDR3 | 160 | MPSFITLASLSTXEGYFDF |
| CONSENSUS | | X = W, F, Y, I, V, L |
| 14A6 L-CDR2 | 161 | YAX₁X₂LQT |
| CONSENSUS | | X₁ = N, S, T, G, D |
| | | X₂ = S, N, S, T, Y, Q |
| 14A6 L-CDR3 | 162 | QQYYSGXT |
| CONSENSUS | | X = W, F, Y, I, V, L |
| 14A6 VH | 163 | EVQLQXISGX₂GLX₃KPX₄X₅X₆LSLTCX₇VX₈GX₃₀SIX₃₁S |
| PARENTAL | | DYWGWX₉RX₁₀X₁₁PGX₁₂X₁₃X₁₄EWX₁₅GFITYSGSTSYN |
| CONSENSUS | | PSLKSRX₁₆X₁₇₁X₁₈X₁₉DTSKNQFX₂₀LX₂₁LX₂₂SVTX₂₃X₂₄ |
| | | DTAX₂₅Y |
| | | X₂₆CARMPSFITLASLSTX₂₇EGYFDFWGX₃₂GTX₂₈X₂₉TV |
| | | SS |
| | | X₁ = E or Q |
| | | X₂ = P or A |
| | | X₃ = V or L |
| | | X₄ = S or P |
| | | X₅ = Q or E or G |
| | | X₆ = S or T |
| | | X₇ = S or T or A |
| | | X₈ = T or S |
| | | X₉ = I or V |
| | | X₁₀ = K or Q |
| | | X₁₁ = F or P |
| | | X₁₂ = N or K |
| | | X₁₃ = K or G |
| | | X₁₄ = M or L |
| | | X₁₅ = M or I |
| | | X16 = I or V |
| | | X17 = S or T |
| | | X18 = T or S |
| | | X19 = R or V |
| | | X20 = F or S |
| | | X21 = Q or K |
| | | X22 = H or S |
| | | X23 = T or A |
| | | X24 = D or A |
| | | X25 = T or V |
| | | X26 = S or Y, |
| | | X27 = W, F, Y, I, V or L |
| | | X28 = M, V, L, A, R, N, P, Q, E, G, I, |
| | | H, K, F, S, T, W or Y |
| | | X₂₉ = V, T or L |
| | | X₃₀ = S or G or Y |
| | | X₃₁ = A or S |
| | | X₃₂ = P or Q |
| 14A6 VH HUMANIZED | 164 | EVQLQX₁SGX₂GLX₃KPX₄XSTLSLTCX₆VX₇GX₈SIX₉SD |
| CONSENSUS | | YWGWX₁₀RX₁₁X₁₂PGKX₁₃X₁₄EWX₁₅GFITYSGSTSYNPS |
| | | LKSRX₁₆TISX₁₇DTSKNQFSLKLX₁₈SVTAX₁₉DTAVYYC |
| | | ARMPSFITLASLSTX₂₀EGYFDFWGQGTX₂₁X₂₂TVSS |
| | | X₁ = E or Q |
| | | X₂ = P or A |
| | | X₃ = V or L |
| | | X₄ = S or P |
| | | X₅ = E or G |
| | | X₆ = T or A or S |
| | | X₇ = S or T |
| | | X₈ = G or S or Y |
| | | X₉ = S or A |
| | | X₁₀ = I or V |
| | | X₁₁-Q or K |
| | | X₁₂ = P or F |
| | | X₁₃ = G or K |
| | | X₁₄ = L or M |
| | | X₁₅ = I or M |
| | | X₁₆ = V or I |
| | | X₁₇ = V or R |
| | | X₁₈ = S or H |
| | | X₁₉ = A or D |
| | | X₂₀ = W, F, Y, I, V, L |
| | | X₂₁ = M, V, L, A, R, N, P, Q, E, G, I, |
| | | H, K, F, S, T, W or Y |
| | | X₂₂ = V, T or L |
| 14A6 VL | 165 | DIQMTQSPSX₁LSASVGDRVTX₂X₃CKASQSIHKNLAW |
| PARENTAL | | YQQKX₄GX₃X₁₅PKX₆LIYYAX₇X₈LQTGX₉PSRFSGSGSG |
| CONSENSUS | | TDFTLTISX₁₀LQPEDX₁₁ATYX₁₂CQQYYSGX₁₃TFGGGT |
| | | KVEX₁₄K |
| | | X₁ = L or S |
| | | X₂ = L or I |
| | | X₃ = N or T |
| | | X₄ = L or P |
| | | X₅ = E or K |
| | | X₆ = F or L |
| | | X₇ = N, S, T, G or D |
| | | X₈ = S, N, T, Y or Q |
| | | X₉ = I or V |
| | | X₁₀ = G or S |
| | | X₁₁ = V or F |
| | | X₁₂ = F or Y |
| | | X₁₃ = W, F, Y, I, V or L |
| | | X₁₄ = L or I |
| | | X₁₅ = A or V |
| 14A6 VL HUMANIZED | 166 | DIQMTQSPSSLSASVGDRVTITCKASQSIHKNLAWYQ |
| CONSENSUS | | QKPGKX₆PKX₁LIYYAX₂X₃LQTGX₄PSRFSGSGSGTDFT |
| | | LTISSLQPEDX₇ATYYCQQYYSGX₅TFGGGTKVEIK |
| | | X₁ = L or F |
| | | X₂ = N, S, T, G or D |
| | | X₃ = S, N, T, Y or Q |
| | | X₄ = V or I |
| | | X₅ = W, F, Y, I, V or L |
| | | X₆ = A or V |
| | | X₇ = F or V |
| 31C6 H-CDR2 | 167 | YIDPYNX₁X₂AKYX₃X₄KFX₅G |
| CONSENSUS | | X₁ = D, R, L, K, F, S, Y or V |
| | | X₂ = G, R, N, Q, E, L K, S, Y or V |
| | | X₃ = N, A or S |
| | | X₄ = E or Q |
| | | X₅ = K or Q |
| 31C6 L-CDR2 | 168 | XIX₂KTLAE |
| CONSENSUS | | X₁ = N, A, V, W, S, T, R, H G, I or V |
| | | X₂ = A, N, I, L, T or V |
| 31C6 VH | 169 | EVQLX₁QSGX₂EX₃X₄KPGX₅SVKX₆SCKASGYTFSSYV |
| PARENTAL | | MHWVX₇QX₈PGQX₉LEWIGYIDPYN |
| CONSENSUS | | X₁₀X₁₁AKYX₁₂X₁₃KFX₁₄GX₁₅X₁₆TLTX₁₇DX₁₈SX₁₉STX₂₀Y |
| | | MELSX₂₁LX₂₂SX₂₃DX₂₄X₂₅VYYCARGGPYGX₂₆YFDVW |
| | | GX₂₇GTTVTVSS |
| | | X₁ = Q or V |
| | | X₂ = P or A |
| | | X₃ = V or L |
| | | X₄ = V or K |
| | | X₅ = S or A |
| | | X₆ = M or V |
| | | X₇ = K or R |
| | | X₈ = K or A |
| | | X₉ = G or R |
| | | X₁₀ = D, R, L, K, F, S, Y or V |
| | | X₁₁ = G, R, N, Q, E, LK, S, Y or V |
| | | X₁₂ = N, A or S |
| | | X₁₃ = E or Q |
| | | X₁₄ = K or Q |
| | | X₁₅ = R or K |
| | | X₁₆ = A or V |
| | | X₁₇ = S or R |
| | | X₁₈ = K or T |
| | | X₁₉ = S, I, A or T |
| | | X₂₀ = A or V |
| | | X₂₁ = R or S |
| | | X₂₂ = T or R |
| | | X₂₃ = D or E |
| | | X₂₄ = S or T |
| | | X₂₅ = A or V |
| | | X₂₆ = W, A, D, E, F, G, I, K, N, Q, R, S, T, V or Y |
| | | X₂₇ = A or Q |
| 31C6 VH | 170 | EVQLVQSGAEVKKPGX₁SVKVSCKASGYTFSSYVMH |
| HUMANIZED | | WVRQAPGQX₂LEWIG |
| CONSENSUS | | YIDPYNX₃X₄AKYX₅X₆KFX₇RVTLTX₈DX₉SX₁₀STX₁₁Y |
| | | MELSX₁₂LRSX₁₃DT |
| | | X₁₄VYYCARGGPYGX₁₅YFDVWGQGTTVTVSS |
| | | X₁ = A or S |
| | | X₂ = R or G |
| | | X₃ = D, R, L, K, F, S, Y or V |
| | | X₄ = G, R, N, Q, E, L K, S, Y or V |
| | | X₅ = N, A or S |
| | | X₆ = E or Q |
| | | X₇ = K or Q |
| | | X₈ = R or S |
| | | X₉ = T or K |
| | | X₁₀ = A, T or I |
| | | X₁₁ = A or V |
| | | X₁₂ = S or R |
| | | X₁₃ = E or D |
| | | X₁₄ = A or V |
| | | X₁₅ = W, A, D, E, F, G, I, K, N, Q, R, S, T, V or Y |
| 31C6 VL | 171 | DIQMTQSPX₁SLSASVGX₂X₃VTITCRASEHIYSYLSWY |
| PARENTAL | | QQKX₄GKX₅PX₆LLX₇YX₈X₉KTLAEGVPSRFSGSGSGT |
| CONSENSUS | | X₁₀FX₁₁LX₁₂IX₁₃SLQPEDX₁₄X₁₅TYYCQHHFGSPLTF |
| | | GX₁₆GTX₁₇LEX₁₈K |
| | | X₁ = A or S |
| | | X₂ = E or D |
| | | X₃ = T or R |
| | | X₄ = Q or P |
| | | Xs = S, A or V |
| | | X₆ = Q or K |
| | | X₇ = V or I |
| | | X₈ = N, A, Y, W, S, T, I or V |
| | | X₉ = A, N, I, L, T or V |
| | | X₁₀ = Q or D |
| | | X₁₁ = S or T |
| | | X₁₂ = K or T |
| | | X₁₃ = N or S |
| | | X₁₄ = F or V |
| | | X₁₅ = G or A |
| | | X₁₆ = A or Q |
| | | X₁₇ = T or R |
| | | X₁₈ = L or I |
| 31C6 L-VL | 172 | DIQMTQSPSSLSASVGDRVTITCRASEHIYSYLSWYQQ |
| HUMANIZED | | KPGKX₁PKLLIY |
| CONSENSUS | | X₂X₃KTLAEGVPSRFSGSGSGTX₄FTLTISSLQPEDX₅AT |
| | | YYCQHHFGSPLTFGQGTRLEIK |
| | | X₁ = A or V |
| | | X₂ = N, A, Y, W, S, T, I or V |
| | | X₃ = A, N, I, L, T or V |
| | | X₄ = D or Q |
| | | X₅ = F or V |
| 31C6 H-CDR3 | 173 | GGPYGXYFDV |
| CONSENSUS | | X = W, A, D, E, F, G, I, K, N, Q, R, S, T, V or Y |
| 31C6 H-CDR3 | 174 | GGPYGAYFDV |
| VARIANT | | |
| 31C6 H-CDR3 | 175 | GGPYGDYFDV |
| VARIANT | | |
| 31C6 H-CDR3 | 176 | GGPYGEYFDV |
| VARIANT | | |
| 31C6 H-CDR3 | 177 | GGPYGFYFDV |
| VARIANT | | |
| 31C6 H-CDR3 | 178 | GGPYGGYFDV |
| VARIANT | | |
| 31C6 H-CDR3 | 179 | GGPYGIYFDV |
| VARIANT | | |
| 31C6 H-CDR3 | 180 | GGPYGKYFDV |
| VARIANT | | |
| 31C6 H-CDR3 | 181 | GGPYGNYFDV |
| VARIANT | | |
| 31C6 H-CDR3 | 182 | GGPYGQYFDV |
| VARIANT | 183 | GGPYGRYFDV |
| 31C6 H-CDR3 | 184 | GGPYGSYFDV |
| VARIANT | 185 | GGPYGTYFDV |
| 31C6 H-CDR3 | 186 | GGPYGVYFDV |
| VARIANT | 187 | GGPYGYYFDV |
| Vibostolimab, light chain | 294 | DIQMTQSPSSLSASVGDRVTITCRASEHIYSYLSWYQQ |
| | | KPGKVPKLLIYNAKTLAEGVPSRFSGSGSGTDFTLTIS |
| | | SLQPEDVATYYCQHHFGSPLTFGQGTRLEIKRTVAAPS |
| | | VFIFPPSDEQLKSGTASVVCLLNNFYPREAKVQWKVD |
| | | NALQSGNSQESVTEQDSKDSTYSLSSTLTLSKADYEK |
| | | HKVYACEVTHQGLSSPVTKSFNRGEC |
| Vibostolimab, heavy chain | 295 | EVQLVQSGAEVKKPGSSVKVSCKASGYTFSSYVMHW |
| | | VRQAPGQGLEWIGYIDPYNDGAKYAQKFQGRVTLTS |
| | | DKSTSTAYMELSSLRSEDTAVYYCARGGPYGWYFDV |
| | | WGQGTTVTVSSASTKGPSVFPLAPSSKSTSGGTAALG |
| | | CLVKDYFPEPVTVSWNSGALTSGVHTFPAVLQSSGLY |
| | | SLSSVVTVPSSSLGTQTYICNVNHKPSNTKVDKKVEP |
| | | KSCDKTHTCPPCPAPELLGGPSVFLFPPKPKDTLMIS |
| | | RTPEVTCVVVDVSHEDPEVKFNWYVDGVEVHNAKTK |
| | | PREEQYNSTYRVVSVLTVLHQDWLNGKEYKCKVSNK |
| | | ALPAPIEKTISKAKGQPREPQVYTLPPSRDELTKNQVS |
| | | LTCLVKGFYPSDIAVEWESNGQPENNYKTTPPVLDSD |
| | | GSFFLYSKLTVDKSRWQQGNVFSCSVMHEALHNHYT |
| | | QKSLSLSPGK |

In some embodiments, an anti-TIGIT antibody or antigen binding fragment thereof comprises three light chain CDRs of CDRL1, CDRL2, and CDRL3 and/or three heavy chain CDRs of CDRH1, CDRH2, and CDRH3.

In one embodiment, the anti-TIGIT antibody or antigen binding fragment thereof comprises a CDRH1 comprising the amino acid sequence as set forth in SEQ ID NO:35, a CDRH2 comprising the amino acid sequence as set forth in SEQ ID NO:36, a CDRH3 comprising any of the amino acid sequences as set forth in SEQ ID NOs:37, 103, 104, 105, 106, 107, or 160, a CDRL1 comprising the amino acid sequence as set forth in SEQ ID NO:38, a CDRL2 comprising any of the amino acid sequences as set forth in SEQ ID NOs:39, 89, 90, 91, 92, 93, 94, 95, 96, 97, or 161, and a CDRL3 comprising any of the amino acid sequences as set forth in SEQ ID NOs:40, 98, 99, 100, 101, 102, or 162.

In another embodiment, the anti-TIGIT antibody or antigen binding fragment thereof comprises a CDRH1 comprising the amino acid sequence as set forth in SEQ ID NO:81, a CDRH2 comprising the amino acid sequence as set forth in SEQ ID NO:82, a CDRH3 comprising the amino acid sequence as set forth in SEQ ID NO:83, a CDRL1 comprising the amino acid sequence as set forth in SEQ ID NO:84, a CDRL2 comprising the amino acid sequence as set forth in SEQ ID NO:85, and a CDRL3 comprising the amino acid sequence as set forth in SEQ ID NO:86.

In another embodiment, the anti-TIGIT antibody or antigen binding fragment thereof comprises a CDRH1 comprising the amino acid sequence as set forth in SEQ ID NO:108, a CDRH2 comprising any of the amino acid sequences as set forth in SEQ ID NOs:109, 116, 117, 118, 119, 120, 121, 122, 123, 124, 125, 126, 127, 128, 129, 130, 131, 154, 155 or 167, a CDRH3 comprising any of the amino acid sequences as set forth in SEQ ID NOs:110, 173, 174, 175, 176, 177, 178, 179, 180, 181, 182, 183, 184, 185, 186 or 187, a CDRL1 comprising the amino acid sequence as set forth in SEQ ID NO:111, a CDRL2 comprising any of the amino acid sequences as set forth in SEQ ID NOs:112, 132, 133, 134, 135, 136, 137, 138, 139, 140, 141, 142 or 168, and a CDRL3 comprising the amino acid sequence as set forth in SEQ ID NO:113.

In one embodiment, the anti-TIGIT antibody or antigen binding fragment thereof comprises a CDRH1 comprising the amino acid sequence as set forth in SEQ ID NO:35, a CDRH2 comprising the amino acid sequence as set forth in SEQ ID NO:36, a CDRH3 comprising the amino acid sequence as set forth in SEQ ID NO:37, a CDRL1 comprising the amino acid sequence as set forth in SEQ ID NO:38, a CDRL2 comprising the amino acid sequence as set forth in SEQ ID NO:39, and a CDRL3 comprising the amino acid sequence as set forth in SEQ ID NO:40.

In one embodiment, the anti-TIGIT antibody or antigen binding fragment thereof comprises CDRH1 comprising the amino acid sequence as set forth in SEQ ID NO:108, a CDRH2 comprising any one of the amino acid sequences as set forth in SEQ ID NO:109, 154 or 145, a CDRH3 comprising the amino acid sequence as set forth in SEQ ID NO:110, a CDRL1 comprising the amino acid sequence as set forth in SEQ ID NO:111, a CDRL2 comprising the amino acid sequence as set forth in SEQ ID NO:112, and a CDRL3 comprising the amino acid sequence as set forth in SEQ ID NO:113.

In another embodiment, the anti-TIGIT antibody or antigen binding fragment thereof comprises a CDRH1 comprising the amino acid sequence as set forth in SEQ ID NO:108, a CDRH2 comprising the amino acid sequence as set forth in SEQ ID NO: 154, a CDRH3 comprising the amino acid sequence as set forth in SEQ ID NO:110, a CDRL1 comprising the amino acid sequence as set forth in SEQ ID NO:111, a CDRL2 comprising the amino acid sequence as set forth in SEQ ID NO: 112, and a CDRL3 comprising the amino acid sequence as set forth in SEQ ID NO:113.

In some embodiments, the anti-TIGIT antibody or antigen binding fragment thereof comprises a light chain comprising or consisting of an amino acid sequence as set forth in SEQ ID NO:294 and a heavy chain comprising or consisting of an amino acid sequence as set forth in SEQ ID NO:295.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising the amino acid sequence as set forth in SEQ ID NO:41 and a variable light chain region comprising the amino acid sequence as set forth in SEQ ID NO:42.

In one embodiment, the anti-TIGIT antibody or antigen binding fragment thereof comprises a variable heavy chain region comprising the amino acid sequence as set forth in SEQ ID NO:87 and a variable light chain region comprising the amino acid sequence as set forth in SEQ ID NO:88.

In one embodiment, the anti-TIGIT antibody or antigen binding fragment thereof comprises a variable heavy chain region comprising the amino acid sequence as set forth in SEQ ID NO: 114 and a variable light chain region comprising the amino acid sequence as set forth in SEQ ID NO:115.

In one embodiment, the anti-TIGIT antibody or antigen binding fragment thereof comprises a variable heavy chain region comprising any of the amino acid sequences as set forth in SEQ ID NOs: 43-58, 65-75 and 87 and a variable light chain region comprising any one of the amino acid sequences as set forth in SEQ ID NOs: 59-64, 76-80 and 88.

In one embodiment, the anti-TIGIT antibody or antigen binding fragment thereof comprises a variable heavy chain region comprising any of the amino acid sequences as set forth in SEQ ID NOs: 144-149 and a variable light chain region comprising any of the amino acid sequences as set forth in SEQ ID NOs: 150-153.

In one embodiment, the anti-TIGIT antibody or antigen binding fragment thereof comprises a variable heavy chain region comprising a variable heavy chain region comprising the amino acid sequence as set forth in SEQ ID NO:148 and a variable light chain region comprising the amino acid sequence as set forth in SEQ ID NO:152.

In one embodiment, the anti-TIGIT antibody or antigen binding fragment thereof comprises a variable heavy chain region comprising the amino acid sequence as set forth in SEQ ID NO:147 and a variable light chain region comprising the amino acid sequence as set forth in SEQ ID NO:150.

In one embodiment, the anti-TIGIT antibody or antigen binding fragment thereof comprises a variable heavy chain region comprising the amino acid sequence as set forth in SEQ ID NO:148 and a variable light chain region comprising the amino acid sequence as set forth in SEQ ID NO:153.

In one embodiment, the anti-TIGIT antibody or antigen binding fragment thereof comprises a variable heavy chain region comprising the amino acid sequence as set forth in SEQ ID NO:163 and a variable light chain region comprising the amino acid sequence as set forth in SEQ ID NO:165.

In one embodiment, the anti-TIGIT antibody or antigen binding fragment thereof comprises a variable heavy chain region comprising the amino acid sequence as set forth in SEQ ID NO:169 and a variable light chain region comprising the amino acid sequence as set forth in SEQ ID NO:171.

In one embodiment, the anti-TIGIT antibody or antigen binding fragment thereof comprises a variable heavy chain region comprising the amino acid sequence as set forth in SEQ ID NO:164 and a variable light chain region comprising the amino acid sequence as set forth in SEQ ID NO:166.

In one embodiment, the anti-TIGIT antibody or antigen binding fragment thereof comprises a variable heavy chain region comprising the amino acid sequence as set forth in SEQ ID NO:170 and a variable light chain region comprising the amino acid sequence as set forth in SEQ ID NO:172.

TABLE 7
Exemplary sequences of anti-TIGIT antibodies

| | SEQ | |
|---|---|---|
| | ID | |
| Description | NO | SEQUENCE |
| 14D7 H-CDR1 | 188 | GAWMD |
| 14D7 H-CDR2 | 189 | EIRTKVNNHATNYGESVKG |
| 14D7 H-CDR3 | 190 | ALYDGFYFDY |
| 14D7 L-CDR1 | 191 | SASSSVSSGYLY |
| 14D7 L-CDR2 | 192 | GTSTLAS |
| 14D7 L-CDR3 | 193 | HQWSSFPYT |
| 14D7 VH | 194 | EVKLEESGGGLVQPGGSMKLSCVASGFTFSGAWMDWVRQSPEKGLEW |
| PARENTAL | | VAEIRTKVNNHATNYGESVKGRFTISRDDSKSSVYLQMNNLRAEDSGIY |
| | | YCRGALYDGFYFDYWGQGTTLTVSS |
| 14D7 VL | 195 | QIVLTQSPAIMSASPGEKVNLTCSASSSVSSGYLYWYQQKPGSSPKLWIY |
| PARENTAL | | GTSTLASGVPARFSGSGSGTSYSLTISNMEAEDAASYFCHQWSSFPYTFG |
| | | GGTKLEMK |
| Hu14D7 VH | 196 | EVQLVESGGGLVQPGGSLKLSCAASGFTFSGAX₁X₂DWVRQAPGKGLEW |
| humanized | | VAEIRTKVNNHATNYGESVKGRFTISRDX₃SKX₄X₅VYLQX₆X₇X₈LRAED |
| consensus | | X₉AVYYCRGALYX₁₀X₁₁FYFDYWGQGTLVTVSS |
| sequence | | X₁ = W, A, R, N, D, Q, E, G, H, I, L, K, F, P, S, T, Y, V |
| | | X₂ = M, V, L, I, G, A, S, T |
| | | X₃ = D, A, R, N, Q, E, G, H, I, L, K, F, S, T, Y, V |
| | | X₄ = S, N |
| | | X₅ = T, S |
| | | X₆ = M, L |
| | | X₇ = N, S |
| | | X₈ = S, N |
| | | X₉ = T, S |
| | | X₁₀ = D, A, R, N, Q, E, G, H, I, L, K, F, P, S, T, W, Y, V |
| | | X₁₁ = G, A, R, N, D, Q, E, H, I, L, K, F, P, S, T, W, Y, V |
| Hu14D7 VH1 | 197 | EVQLVESGGGLVQPGGSLKLSCAASGFTFSGAWMDWVRQAPGKGLEW |
| (Humanized VH | | VAEIRTKVNNHATNYGESVKGRFTISRDDSKSTVYLQMNSLRAEDTAVY |
| chain) | | YCRGALYDGFYFDYWGQGTLVTVSS |
| Hu14D7 VH2 | 198 | EVQLVESGGGLVQPGGSLKLSCAASGFTFSGAWMDWVRQAPGKGLEWV |
| (Humanized VH | | AEIRTKVNNHATNYGESVKGRFTISRDDSKSSVYLQMNSLRAEDTAVY |
| chain) | | YCRGALYDGFYFDYWGQGTLVTVSS |
| Hu14D7 VH3 | 199 | EVQLVESGGGLVQPGGSLKLSCAASGFTFSGAWMDWVRQAPGKGLEW |
| (Humanized VH | | VAEIRTKVNNHATNYGESVKGRFTISRDDSKNTVYLQMNSLRAEDTAVY |
| chain) | | YCRGALYDGFYFDYWGQGTLVTVSS |
| Hu14D7 VL | 200 | EIVLTQSPATLSLSPGERAX₁LSCSASSSVSSGYLYWYQQKPGQAPX₇LX₂I |
| humanized | | YGTSTLASGX₈PARFSGSGSGTDYTLTISSX₃EPEDX₄AVYYCHQX₅SSFPY |
| consensus | | TFGQGTKLEX₆K |
| Sequence | | X₁ = T, S |
| | | X₂ = W, A, R, N, D, Q, E, G, H, I, L, K, F, P, S, T, Y, V |
| | | X₃ = L, V, I |
| | | X₄ = F, V, L, I, T |
| | | X₅ = W, A, R, N, D, Q, E, G, H, I, L, K, F, P, S, T, Y, V |
| | | X₆ = I, L |
| | | X₇ = K, R |
| | | X₈ = V, I |
| Hu14D7VL1 | 201 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQWSSFPYTFGQ |
| chain) | | GTKLEIK |
| Hu14D7VL2 | 202 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQWSSFPYTFGQ |
| chain) | | GTKLEIK |
| Hu14D7VL3 | 203 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQWSSFPYTFGQ |
| chain) | | GTKLEIK |
| 26B10 H-CDR1 | 204 | EFTMH |
| 26B10 H-CDR2 | 205 | GLKPDNGGISYNQKFKG |
| 26B10 H-CDR3 | 206 | GAYYRYDADY |
| 26B10 L-CDR1 | 207 | KASQDVKTAVA |
| 26B10 L-CDR2 | 208 | SASYRNT |
| 26B10 L-CDR3 | 209 | QQHYSTPFT |
| 26B10 VH | 210 | EVQLQQSGPELVKPGASVKISCKTSGYTFTEFTMHWVKQSHGKSLEWIG |
| PARENTAL | | GLKPDNGGISYNQKFKGRATLAVDKSSNTAYMELRSLTSEDSAVYYCAR |
| | | GAYYRYDADYWGQGTTLTVSS |
| 26B10 VL | 211 | DIVLTQSHKFMSTSVGDRVSITCKASQDVKTAVAWYQQKSGQSPKLLIY |
| PARENTAL | | SASYRNTGVPDRFTGSGSGTDFTFTIDSVQAEDLAVYFCQQHYSTPFTFG |
| | | TGTKLELK |
| 26B10 VH | 212 | EVQLVQSGAEVKKPGASVKISCKXISGYTFTEFTX₂HWVX₃QAPGKGLEW |
| HUMANIZED | | IGGLKPDX₄X₅GISYNQKFKGRATLTVDX₆STX₇TAYX₈ELSSLRSEDX₉AV |
| CONSESUS | | YYCARGAYYRYX₁₀X₁₁DYWGQGTLVTVSS |
| Sequence | | X₁ = T, V |
| | | X₂ = M, V, L, I, G, A, S, T |
| | | X₃ = K, R |
| | | X₄ = N, A, R, D, Q, E, G, H, I, L, K, F, P, S, T, W, Y, V |
| | | X₅ = G, A, R, N, D, Q, E, H, I, L, K, F, P, S, T, W, Y, V |
| | | X₆ = k, t, d, s |
| | | X = = N, S |
| | | X₈ = M, V, L, I, G, A, S, T |
| | | X₉ = T, S |
| | | X₁₀ = D, A, R, N, Q, E, G, H, I, L, K, F, P, S, T, W, Y, V |
| | | X₁₁ = A, R, N, D, Q, E, G, H, I, L, K, F, P, S, T, W, Y, V, M |
| Hu26B10 VH1 | 213 | EVQLVQSGAEVKKPGASVKISCKTSGYTFTEFTMHWVKQAPGKGLEWI |
| (Humanized VH | | GGLKPDNGGISYNQKFKGRATLTVDKSTNTAYMELSSLRSEDTAVYYCA |
| chain) | | RGAYYRYDADYWGQGTLVTVSS |
| Hu26B10 VH2 | 214 | EVQLVQSGAEVKKPGASVKISCKTSGYTFTEFTMHWVRQAPGKGLEWIG |
| (Humanized VH | | GLKPDNGGISYNQKFKGRATLTVDKSTSTAYMELSSLRSEDTAVYYCAR |
| chain) | | GAYYRYDADYWGQGTLVTVSS |
| Hu26B10 VH3 | 215 | EVQLVQSGAEVKKPGASVKISCKVSGYTFTEFTMHWVRQAPGKGLEWI |
| (Humanized VH | | GGLKPDNGGISYNQKFKGRATLTVDTSTSTAYMELSSLRSEDTAVYYCA |
| chain) | | RGAYYRYDADYWGQGTLVTVSS |
| 26B10 VL | 216 | DIQLTQSPSSLSASVGDRVTITCKASQDVKTAVAWYQQKPGKAPKLLIYS |
| HUMANIZED | | ASYRX₁X₂GVP |
| CONSESUS | | X₃RFSGSGSGTDFTX₄TISSLQPEDFATYYCQQHYSTPFTFGQGTKLEIK |
| sequence | | X₁ = N, Q, D, E |
| | | X₂ = T, S, A |
| | | X₃ = D, S |
| | | X₄ = F, L |
| Hu26B10 VL1 | 217 | DIQLTQSPSSLSASVGDRVTITCKASQDVKTAVAWYQQKPGKAPKLLIYS |
| (Humanized VL | | ASYRNTGVPDRFSGSGSGTDFTFTISSLQPEDFATYYCQQHYSTPFTFGQG |
| chain) | | TKLEIK |
| Hu26B10 VL2 | 218 | DIQLTQSPSSLSASVGDRVTITCKASQDVKTAVAWYQQKPGKAPKLLIYS |
| (Humanized VL | | ASYRNTGVPSRFSGSGSGTDFTFTISSLQPEDFATYYCQQHYSTPFTFGQG |
| chain) | | TKLEIK |
| Hu26B10 VL3 | 219 | DIQLTQSPSSLSASVGDRVTITCKASQDVKTAVAWYQQKPGKAPKLLIYS |
| (Humanized VL | | ASYRNTGVPSRFSGSGSGTDFTLTISSLQPEDFATYYCQQHYSTPFTFGQG |
| chain) | | TKLEIK |
| 14D7H-CDR3 | 22 | ALYEGFYFDY |
| (D104E) | | |
| 14D7H-CDR3 | 221 | ALYDAFYFDY |
| (G105A) | | |
| 14D7H-CDR3 | 222 | ALYDSFYFDY |
| (G105S) | | |
| Hu14D7 VH1 | 223 | EVQLVESGGGLVQPGGSLKLSCAASGFTFSGAWMDWVRQAPGKGLEW |
| (Humanized VH | | VAEIRTKVNNHATNYGESVKGRFTISRDDSKSTVYLQMNSLRAEDTAVY |
| chain) (D104E) | | YCRGALYEGFYFDYWGQGTLVTVSS |
| Hu14D7 VH1 | 224 | EVQLVESGGGLVQPGGSLKLSCAASGFTFSGAWMDWVRQAPGKGLEW |
| (Humanized VH | | VAEIRTKVNNHATNYGESVKGRFTISRDDSKSTVYLQMNSLRAEDTAVY |
| chain) (G105A) | | YCRGALYDAFYFDYWGQGTLVTVSS |
| Hu14D7 VH1 | 225 | EVQLVESGGGLVQPGGSLKLSCAASGFTFSGAWMDWVRQAPGKGLEW |
| (Humanized VH | | VAEIRTKVNNHATNYGESVKGRFTISRDDSKSTVYLQMNSLRAEDTAVY |
| chain) (G105S) | | YCRGALYDSFYFDYWGQGTLVTVSS |
| Hu14D7 VH2 | 226 | EVQLVESGGGLVQPGGSLKLSCAASGFTFSGAWMDWVRQAPGKGLEW |
| (Humanized VH | | VAEIRTKVNNHATNYGESVKGRFTISRDDSKSSVYLQMNSLRAEDTAVY |
| chain) (D104E) | | YCRGALYDEFYFDYWGQGTLVTVSS |
| Hu14D7 VH2 | 227 | EVQLVESGGGLVQPGGSLKLSCAASGFTFSGAWMDWVRQAPGKGLEW |
| (Humanized VH | | VAEIRTKVNNHATNYGESVKGRFTISRDDSKSSVYLQMNSLRAEDTAVY |
| chain) (G105A) | | YCRGALYDAFYFDYWGQGTLVTVSS |
| Hu14D7 VH2 | 228 | EVQLVESGGGLVQPGGSLKLSCAASGFTFSGAWMDWVRQAPGKGLEW |
| (Humanized VH | | VAEIRTKVNNHATNYGESVKGRFTISRDDSKSSVYLQMNSLRAEDTAVY |
| chain) (G105S) | | YCRGALYDSFYFDYWGQGTLVTVSS |
| Hu14D7 VH3 | 229 | EVQLVESGGGLVQPGGSLKLSCAASGFTFSGAWMDWVRQAPGKGLEW |
| (Humanized VH | | VAEIRTKVNNHATNYGESVKGRFTISRDDSKNTVYLQMNSLRAEDTAVY |
| chain) (D104E) | | YCRGALYEGFYFDYWGQGTLVTVSS |
| Hu14D7 VH3 | 230 | EVQLVESGGGLVQPGGSLKLSCAASGFTFSGAWMDWVRQAPGKGLEW |
| (Humanized VH | | VAEIRTKVNNHATNYGESVKGRFTISRDDSKNTVYLQMNSLRAEDTAVY |
| chain) (G105A) | | YCRGALYDAFYFDYWGQGTLVTVSS |
| Hu14D7 VH3 | 231 | EVQLVESGGGLVQPGGSLKLSCAASGFTFSGAWMDWVRQAPGKGLEW |
| (Humanized VH | | VAEIRTKVNNHATNYGESVKGRFTISRDDSKNTVYLQMNSLRAEDTAVY |
| chain) (G105S) | | YCRGALYDSFYFDYWGQGTLVTVSS |
| 14D7 L-CDR3 | 232 | HQASSFPYT |
| (W92A) | | |
| 14D7 L- | 233 | HQDSSFPYT |
| CDR3(W92D) | | |
| 14D7 L- | 234 | HQESSFPYT |
| CDR3(W92E) | | |
| 14D7 L- | 235 | HQFSSFPYT |
| CDR3(W92F) | | |
| 14D7 L- | 236 | HQGSSFPYT |
| CDR3(W92G) | | |
| 14D7L-CDR3 | 237 | HQHSSFPYT |
| (W92H) | | |
| Hu14D7VL1 | 238 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQASSFPYTFGQ |
| chain) (W92A) | | GTKLEIK |
| Hu14D7VL1 | 239 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQDSSFPYTFGQ |
| chain) (W92D) | | GTKLEIK |
| Hu14D7VL1 | 240 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQESSFPYTFGQ |
| chain) (W92E) | | GTKLEIK |
| Hu14D7VL1 | 241 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQFSSFPYTFGQ |
| chain) (W92F) | | GTKLEIK |
| Hu14D7VL1 | 242 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQGSSFPYTFGQ |
| chain) (W92G) | | GTKLEIK |
| Hu14D7VL1 | 243 | EIVLTQSPATLSLSPGERATLSCSASSSVSSGYLYWYQQKPGQAPKLWIY |
| (Humanized VL | | GTSTLASGVPARFSGSGSGTDYTLTISSLEPEDFAVYYCHQHSSFPYTFGQ |
| chain) (W92H) | | GTKLEIK |
| Hu14D7VL2 | 244 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQASSFPYTFGQ |
| chain) (W92A) | | GTKLEIK |
| Hu14D7VL2 | 245 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQDSSFPYTFGQ |
| chain) (W92D) | | GTKLEIK |
| Hu14D7VL2 | 246 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQESSFPYTFGQ |
| chain) (W92E) | | GTKLEIK |
| Hu14D7VL2 | 247 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQFSSFPYTFGQ |
| chain) (W92F) | | GTKLEIK |
| Hu14D7VL2 | 248 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQGSSFPYTFGQ |
| chain) (W92G) | | GTKLEIK |
| Hu14D7VL2 | 249 | EIVLTQSPATLSLSPGERATLSCSASSSVSSGYLYWYQQKPGQAPRLWIY |
| (Humanized VL | | GTSTLASGVPARFSGSGSGTDYTLTISSLEPEDFAVYYCHQHSSFPYTFGQ |
| chain) (W92H) | | GTKLEIK |
| Hu14D7VL3 | 250 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQASSFPYTFGQ |
| chain) (W92A) | | GTKLEIK |
| Hu14D7VL3 | 251 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQDSSFPYTFGQ |
| chain) (W92D) | | GTKLEIK |
| Hu14D7VL3 | 252 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQESSFPYTFGQ |
| chain) (W92E) | | GTKLEIK |
| Hu14D7VL3 | 253 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQFSSFPYTFGQG |
| chain) (W92H) | | TKLEIK |
| Hu14D7VL3 | 254 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQGSSFPYTFGQ |
| chain) (W92G) | | GTKLEIK |
| Hu14D7VL3 | 255 | EIVLTQSPATLSLSPGERATLSCSASSSVSSGYLYWYQQKPGQAPRLWIY |
| (Humanized VL | | GTSTLASGIPARFSGSGSGTDYTLTISSLEPEDFAVYYCHQHSSFPYTFGQ |
| chain) (W92H) | | GTKLEIK |
| 26B10 H-CDR2 | 256 | GLKPDQGGISYNQKFKG |
| (N55Q) | | |
| 26B10 H- | 257 | GLKPDDGGISYNQKFKG |
| CDR2(N55D) | | |
| 26B10 H- | 258 | GLKPDNAGISYNQKFKG |
| CDR2(N56A) | | |
| 26B10 H- | 259 | GLKPDTGGISYNQKFKG |
| CDR2(N55T) | | |
| 26B10 H- | 260 | GLKPDSGGISYNQKFKG |
| CDR2(N55S) | | |
| 26B10 H- | 261 | GLKPDGGGISYNQKFKG |
| CDR2(N55G) | | |
| 26B10 H-CDR2 | 262 | GLKPDNSGISYNQKFKG |
| (G56S) | | |
| 26B10 H- | 263 | GLKPDNTGISYNQKFKG |
| CDR2(G56T) | | |
| Hu26B10 VH1 | 264 | EVQLVQSGAEVKKPGASVKISCKTSGYTFTEFTMHWVKQAPGKGLEWI |
| (Humanized VH | | GGLKPDQGGISYNQKFKGRATLTVDKSTNTAYMELSSLRSEDTAVYYCA |
| chain)(N55Q) | | RGAYYRYDADYWGQGTLVTVSS |
| Hu26B10 VH1 | 265 | EVQLVQSGAEVKKPGASVKISCKTSGYTFTEFTMHWVKQAPGKGLEWI |
| (Humanized VH | | GGLKPDDGGISYNQKFKGRATLTVDKSTNTAYMELSSLRSEDTAVYYCA |
| chain)(N55D) | | RGAYYRYDADYWGQGTLVTVSS |
| Hu26B10 VH1 | 266 | EVQLVQSGAEVKKPGASVKISCKTSGYTFTEFTMHWVKQAPGKGLEWI |
| (Humanized VH | | GGLKPDNAGISYNQKFKGRATLTVDKSTNTAYMELSSLRSEDTAVYYCA |
| chain)(G56A) | | RGAYYRYDADYWGQGTLVTVSS |
| Hu26B10 VH1 | 267 | EVQLVQSGAEVKKPGASVKISCKTSGYTFTEFTMHWVKQAPGKGLEWI |
| (Humanized VH | | GGLKPDTGGISYNQKFKGRATLTVDKSTNTAYMELSSLRSEDTAVYYCA |
| chain)(N55T) | | RGAYYRYDADYWGQGTLVTVSS |
| Hu26B10 VH1 | 268 | EVQLVQSGAEVKKPGASVKISCKTSGYTFTEFTMHWVKQAPGKGLEWI |
| (Humanized VH | | GGLKPDSGGISYNQKFKGRATLTVDKSTNTAYMELSSLRSEDTAVYYCA |
| chain)(N55S) | | RGAYYRYDADYWGQGTLVTVSS |
| Hu26B10 VH1 | 269 | EVQLVQSGAEVKKPGASVKISCKTSGYTFTEFTMHWVKQAPGKGLEWI |
| (Humanized VH | | GGLKPDGGGISYNQKFKGRATLTVDKSTNTAYMELSSLRSEDTAVYYCA |
| chain)(N55G) | | RGAYYRYDADYWGQGTLVTVSS |
| Hu26B10 VH1 | 270 | EVQLVQSGAEVKKPGASVKISCKTSGYTFTEFTMHWVKQAPGKGLEWI |
| (Humanized VH | | GGLKPDNSGISYNQKFKGRATLTVDKSTNTAYMELSSLRSEDTAVYYCA |
| chain)(G56S) | | RGAYYRYDADYWGQGTLVTVSS |
| Hu26B10 VH1 | 271 | EVQLVQSGAEVKKPGASVKISCKTSGYTFTEFTMHWVKQAPGKGLEWI |
| (Humanized VH | | GGLKPDNTGISYNQKFKGRATLTVDKSTNTAYMELSSLRSEDTAVYYCA |
| chain)(G56T) | | RGAYYRYDADYWGQGTLVTVSS |
| Hu26B10 VH2 | 272 | EVQLVQSGAEVKKPGASVKISCKTSGYTFTEFTMHWVRQAPGKGLEWIG |
| (Humanized VH | | GLKPDQGGISYNQKFKGRATLTVDKSTSTAYMELSSLRSEDTAVYYCAR |
| chain)(N55Q) | | GAYYRYDADYWGQGTLVTVSS |
| Hu26B10 VH2 | 273 | EVQLVQSGAEVKKPGASVKISCKTSGYTFTEFTMHWVRQAPGKGLEWIG |
| (Humanized VH | | GLKPDDGGISYNQKFKGRATLTVDKSTSTAYMELSSLRSEDTAVYYCAR |
| chain)(N55D) | | GAYYRYDADYWGQGTLVTVSS |
| Hu26B10 VH1 | 274 | EVQLVQSGAEVKKPGASVKISCKTSGYTFTEFTMHWVRQAPGKGLEWIG |
| (Humanized VH | | GLKPDNAGISYNQKFKGRATLTVDKSTSTAYMELSSLRSEDTAVYYCAR |
| chain)(G56A) | | GAYYRYDADYWGQGTLVTVSS |
| Hu26B10 VH2 | 275 | EVQLVQSGAEVKKPGASVKISCKTSGYTFTEFTMHWVRQAPGKGLEWIG |
| (Humanized VH | | GLKPDTGGISYNQKFKGRATLTVDKSTSTAYMELSSLRSEDTAVYYCAR |
| chain)(N55T) | | GAYYRYDADYWGQGTLVTVSS |
| Hu26B10 VH2 | 276 | EVQLVQSGAEVKKPGASVKISCKTSGYTFTEFTMHWVRQAPGKGLEWIG |
| (Humanized VH | | GLKPDSGGISYNQKFKGRATLTVDKSTSTAYMELSSLRSEDTAVYYCAR |
| chain)(N55S) | | GAYYRYDADYWGQGTLVTVSS |
| Hu26B10 VH2 | 277 | EVQLVQSGAEVKKPGASVKISCKTSGYTFTEFTMHWVRQAPGKGLEWIG |
| (Humanized VH | | GLKPDGGGISYNQKFKGRATLTVDKSTSTAYMELSSLRSEDTAVYYCAR |
| chain)(N55G) | | GAYYRYDADYWGQGTLVTVSS |
| Hu26B10 VH2 | 278 | EVQLVQSGAEVKKPGASVKISCKTSGYTFTEFTMHWVRQAPGKGLEWIG |
| (Humanized VH | | GLKPDNSGISYNQKFKGRATLTVDKSTSTAYMELSSLRSEDTAVYYCAR |
| chain)(G56S) | | GAYYRYDADYWGQGTLVTVSS |
| Hu26B10 VH2 | 279 | EVQLVQSGAEVKKPGASVKISCKTSGYTFTEFTMHWVRQAPGKGLEWIG |
| (Humanized VH | | GLKPDNTGISYNQKFKGRATLTVDKSTSTAYMELSSLRSEDTAVYYCAR |
| chain)(G56T) | | GAYYRYDADYWGQGTLVTVSS |
| Hu26B10 VH3 | 280 | EVQLVQSGAEVKKPGASVKISCKVSGYTFTEFTMHWVRQAPGKGLEWI |
| (Humanized VH | | GGLKPDQGGISYNQKFKGRATLTVDTSTSTAYMELSSLRSEDTAVYYCA |
| chain)(N55Q) | | RGAYYRYDADYWGQGTLVTVSS |
| Hu26B10 VH3 | 281 | EVQLVQSGAEVKKPGASVKISCKVSGYTFTEFTMHWVRQAPGKGLEWI |
| (Humanized VH | | GGLKPDDGGISYNQKFKGRATLTVDTSTSTAYMELSSLRSEDTAVYYCA |
| chain)(N55D) | | RGAYYRYDADYWGQGTLVTVSS |
| Hu26B10 VH3 | 282 | EVQLVQSGAEVKKPGASVKISCKVSGYTFTEFTMHWVRQAPGKGLEWI |
| (Humanized VH | | GGLKPDNAGISYNQKFKGRATLTVDTSTSTAYMELSSLRSEDTAVYYCA |
| chain)(G56A) | | RGAYYRYDADYWGQGTLVTVSS |
| Hu26B10 VH3 | 283 | EVQLVQSGAEVKKPGASVKISCKVSGYTFTEFTMHWVRQAPGKGLEWI |
| (Humanized VH | | GGLKPDTGGISYNQKFKGRATLTVDTSTSTAYMELSSLRSEDTAVYYCA |
| chain)(N55T) | | RGAYYRYDADYWGQGTLVTVSS |
| Hu26B10 VH3 | 284 | EVQLVQSGAEVKKPGASVKISCKVSGYTFTEFTMHWVRQAPGKGLEWI |
| (Humanized VH | | GGLKPDSGGISYNQKFKGRATLTVDTSTSTAYMELSSLRSEDTAVYYCA |
| chain)(N55S) | | RGAYYRYDADYWGQGTLVTVSS |
| Hu26B10 VH3 | 285 | EVQLVQSGAEVKKPGASVKISCKVSGYTFTEFTMHWVRQAPGKGLEWI |
| (Humanized VH | | GGLKPDGGGISYNQKFKGRATLTVDTSTSTAYMELSSLRSEDTAVYYCA |
| chain)(N55G) | | RGAYYRYDADYWGQGTLVTVSS |
| Hu26B10 VH3 | 286 | EVQLVQSGAEVKKPGASVKISCKVSGYTFTEFTMHWVRQAPGKGLEWI |
| (Humanized VH | | GGLKPDNSGISYNQKFKGRATLTVDTSTSTAYMELSSLRSEDTAVYYCA |
| chain)(G56S) | | RGAYYRYDADYWGQGTLVTVSS |
| Hu26B10 VH3 | 287 | EVQLVQSGAEVKKPGASVKISCKVSGYTFTEFTMHWVRQAPGKGLEWI |
| (Humanized VH | | GGLKPDNTGISYNQKFKGRATLTVDTSTSTAYMELSSLRSEDTAVYYCA |
| chain)(G56T) | | RGAYYRYDADYWGQGTLVTVSS |
| hTIGIT epitope | 288 | SSTTAQVNWEQQDQL |
| (24-41) | | |
| hTIGIT epitope | 289 | IYHTYPDGT |
| (85-93) | | |
| hTIGIT epitope | 290 | GRIFL |
| (96-100) | | |

In one embodiment, the anti-TIGIT antibody or antigen binding fragment comprises a CDRH1 comprising the amino acid sequence as set forth in SEQ ID NO:188, a CDRH2 comprising the amino acid sequence as set forth in SEQ ID NO:189, a CDRH3 comprising any of the amino acid sequences as set forth in SEQ ID NOs:190, 220, 221, or 222, a CDRL1 comprising the amino acid sequence as set forth in SEQ ID NO:191, a CDRL2 comprising the amino acid sequence as set forth in SEQ ID NO:192, and a CDRL3 comprising any of the amino acid sequences as set forth in SEQ ID NOs:193, 232, 233, 234, 235, 236, or 237.

In one embodiment, the anti-TIGIT antibody or antigen binding fragment thereof comprises a CDRH1 comprising the amino acid sequence as set forth in SEQ ID NO:204, a CDRH2 comprising any of the amino acid sequences as set forth in SEQ ID NOs: 205, 256, 257, 258, 259, 260, 261, 262, or 263, a CDRH3 comprising the amino acid sequence as set forth in SEQ ID NO:206, a CDRL1 comprising the amino acid sequence as set forth in SEQ ID NO:207, a CDRL2 comprising the amino acid sequence as set forth in SEQ ID NO:208, and a CDRL3 comprising the amino acid sequence as set forth in SEQ ID NO:209.

In one embodiment, the anti-TIGIT antibody or antigen binding fragment thereof comprises a variable heavy chain region and a variable light chain variable region. In one embodiment, the anti-TIGIT antibody or antigen binding fragment thereof comprises a variable heavy chain region comprising the amino acid sequence as set forth in SEQ ID NO:194 and a variable light chain region comprising the amino acid sequence as set forth in SEQ ID NO:195.

In one embodiment, the anti-TIGIT antibody or antigen binding fragment thereof comprises a variable heavy chain region comprising the amino acid sequence as set forth in SEQ ID NO:196 and a variable light chain region comprising the amino acid sequence as set forth in SEQ ID NO:200.

In one embodiment, the anti-TIGIT antibody or antigen binding fragment thereof comprises a variable heavy chain region comprising the amino acid sequence as set forth in SEQ ID NO: 210 and a variable light chain region comprising the amino acid sequence as set forth in SEQ ID NO:211.

In one embodiment, the anti-TIGIT antibody or antigen binding fragment thereof comprises a variable heavy chain region comprising the amino acid sequence as set forth in SEQ ID NO: 212 and a variable light chain region comprising the amino acid sequence as set forth in SEQ ID NO:216.

In one embodiment, the anti-TIGIT antibody or antigen binding fragment thereof comprises a variable heavy chain region comprising any of the amino acid sequences as set forth in SEQ ID NOs: 197, 198, 199, 223, 224, 225, 226, 227, 228, 229, 230, and 231 and a variable light chain region comprising any of the amino acid sequences as set forth in SEQ ID NOs: 201, 202, 203, 238, 239, 240, 241, 242, 243, 244, 245, 246, 247, 248, 249, 250, 251, 252, 253, 254, and 255.

In one embodiment, the anti-TIGIT antibody or antigen binding fragment thereof comprises a variable heavy chain region comprising any of the amino acid sequences as set forth in SEQ ID NOs: 213, 214, 215, 264, 265, 266, 267, 268, 269, 270, 271, 272, 273, 274, 275, 276, 277, 278, 279, 280, 281, 282, 283, 284, 285, and 286 and a variable light chain region comprising any of the amino acid sequences as set forth in SEQ ID NOs: 217, 218, and 219.

Additional anti-TIGIT antibodies which may be used in the formulations described herein include those disclosed, for example, in PCT International Application No. WO 2016/106302; WO 2016/011264; and WO 2009/126688.

TABLE 8
Exemplary Heavy Chain Sequences

| | | |
|---|---|---|
| Heavy chain | 291 | ASTKGPSVFPLAPSSKSTSGGTA |
| constant domain- | | ALGCLVKDYFPEPVTVSWNSGAL |
| IgG1 | | TSGVHTFPAVLQSSGLYSLSSVV |
| | | TVPSSSLGTQTYICNVNHKPSNT |
| | | KVDKKVEPKSCDKTHTCPPCPAP |
| | | ELLGGPSVFLFPPKPKDTLMISR |
| | | TPEVTCVVVDVSHEDPEVKFNWY |
| | | VDGVEVHNAKTKPREEQYNSTYR |
| | | VVSVLTVLHQDWLNGKEYKCKVS |
| | | NKALPAPIEKTISKAKGQPREPQ |
| | | VYTLPPSRDELTKNQVSLTCLVK |
| | | GFYPSDIAVEWESNGQPENNYKT |
| | | TPPVLDSDGSFFLYSKLTVDKSR |
| | | WQQGNVFSCSVMHEALHNHYTQK |
| | | SLSLSPGK |
| Heavy chain | 292 | TKGPSVFPLAPCSRSTSESTAAL |
| constant domain- | | GCLVKDYFPEPVTVSWNSGALTS |
| IgG4 | | GVHTFPAVLQSSGLYSLSSVVTV |
| S228P | | PSSSLGTKTYTCNVDHKPSNTKV |
| | | DKRVESKYGPPCPPCPAPEFLGG |
| | | PSVFLFPPKPKDTLMISRTPEVT |
| | | CVVVDVSQEDPEVQFNWYVDGVE |
| | | VHNAKTKPREEQFNSTYRVVSVL |
| | | TVLHQDWLNGKEYKCKVSNKGLP |
| | | SSIEKTISKAKGQPREPQVYTLP |
| | | PSQEEMTKNQVSLTCLVKGFYPS |
| | | DIAVEWESNGQPENNYKTTPPVL |
| | | DSDGSFFLYSRLTVDKSRWQEGN |
| | | VFSCSVMHEALHNHYTQKSLSLS |
| | | LGK |
| Kappa light | 293 | VAAPSVFIFPPSDEQLKSGTASV |
| chain | | VCLLNNFYPREAKVQWKVDNALQ |
| constant domain | | SGNSQESVTEQDSKDSTYSLSST |
| | | LTLSKADYEKHKVYACEVTHQGL |
| | | SSPVTKSFNRGEC |

In any of the above mentioned embodiments, the anti-TIGIT antibody or antigen binding fragment thereof is an antibody comprising any of the variable heavy chains described above and any human heavy chain constant domain. In one embodiment, the antibody or antigen binding fragment thereof of the invention is of the IgG isotype, and comprises a human IgG1, IgG2, IgG3 or IgG4 human heavy chain constant domain. In one embodiment, the antibody or antigen binding fragment thereof of the invention comprises a human heavy chain IgG1 constant domain (SEQ ID NO: 291) or a variant thereof, wherein the variant comprises up to 20 modified amino acid substitutions. In one embodiment, the antibody or antigen binding fragment thereof of the invention is an antibody comprising a human heavy chain IgG1 constant domain comprising the amino acid sequence as set forth in SEQ ID NO: 291. In one embodiment, the antibody or antigen binding fragment thereof of the invention comprises a human heavy chain IgG1 constant domain wherein the IgG1 constant domain is afucosylated. In one embodiment, the antibody or antigen binding fragment thereof of the invention comprises a human heavy chain IgG4 constant domain or a variant thereof, wherein the variant comprises up to 20 modified amino acid substitutions. In another embodiment, the antibody or antigen binding fragment thereof of the invention comprises a human heavy chain IgG4 constant domain, wherein the amino acid at position 228 (using EU numbering scheme) has been substituted from Ser to Pro. In one embodiment, the antibody or antigen binding fragment thereof of the invention comprises a human heavy chain IgG4 constant domain comprising the amino acid sequence as set forth in SEQ ID NO: 292.

In any of the above mentioned embodiments, the anti-TIGIT antibody or antigen binding fragment thereof can comprise any of the variable light chains described above and human light chain constant domain. In one embodiment, the antibody or antigen binding fragment thereof of the invention comprises a human kappa light chain constant domain or a variant thereof, wherein the variant comprises up to 20 modified amino acid substitutions. In another embodiment, the antibody or antigen binding fragment thereof of the invention comprises a human lambda light chain constant domain or a variant thereof, wherein the variant comprises up to 20 modified amino acid substitutions. In one embodiment, the antibody or antigen binding fragment thereof of the invention comprises a human kappa light chain constant domain comprising the amino acid sequence as set forth in SEQ ID NO: 293.

### HIF-2α Inhibitors

Provided herein are HIF-2α inhibitors that can be used in the various methods, kits, and uses disclosed herein, including any chemical compound or biological molecule that inhibits the activity of HIF-2α.

In some embodiments, the HIF-2α inhibitor is belzutifan or a pharmaceutically acceptable salt thereof, which is also known as MK-6482, PT2977, 3-[(1S,2S,3R)-2,3-difluoro-1-hydroxy-7-methylsulfonyl-indan-4-yl]oxy-5-fluoro-benzonitrile, and 3-[[(1S,2S,3R)-2,3-difluoro-2,3-dihydro-1-hydroxy-7-(methylsulfonyl)-1H-inden-4-yl]oxy]-5-fluorobenzonitrile and has the following chemical structure:

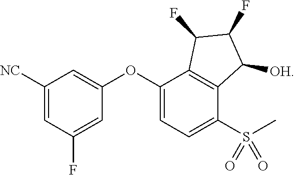 [SMILES: CS(=O)(=O)c1ccc(Oc2cc(F)cc(C#N)c2)c2c1[C@H](O)[C@H](F)[C@@H]2F]

Belzutifan and its synthesis are described in U.S. Pat. No. 9,969,689, which is hereby incorporated by reference in its entirety. Belzutifan as a potential treatment for clear cell renal cell carcinoma is described in Rui Xu et al., J. Med. Chem. 2019, 62, 6876-6893, which is hereby incorporated by reference in its entirety. U.S. Publication 2018-0042884 describes the treatment of glioblastoma with a HIF-2α inhibitor, and is hereby incorporated by reference in its entirety. Oral formulations of belzutifan are described in International Application No. PCT/US2019/57725, which was filed on Oct. 23, 2019, and is hereby incorporated by reference in its entirety.

### Methods of Treating Cancer or Von-Hippel Lindau Disease Using a Combination of a PD-1 Antagonist, a TIGIT Antagonist, and an HIF-2α Inhibitor

In another aspect, provided herein are methods of treating cancer (e.g., RCC) using a combination of a TIGIT antagonist, a PD-1 antagonist, and an HIF-2α inhibitor.

In certain embodiments, the TIGIT antagonist is an anti-TIGIT antibody or antigen binding fragment thereof. In some embodiments, the PD-1 antagonist is an anti-PD-1 antibody or antigen binding fragment thereof.

In certain embodiments, the method of treating cancer comprises administering to a human patient in need thereof:
(a) a TIGIT antagonist;
(b) a PD-1 antagonist; and
(c) belzutifan or a pharmaceutically acceptable salt thereof.

In some embodiments, the cancer is selected from the group consisting of: sarcoma (angiosarcoma, fibrosarcoma, rhabdomyosarcoma, liposarcoma), myxoma, rhabdomyoma, fibroma, lipoma and teratoma; Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma; Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma) colorectal; Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia [acute and chronic], acute lymphoblastic leukemia, chronic lymphocytic leukemia, myeloproliferative diseases, multiple myeloma, myelodysplastic syndrome); hematopoietic tumors of the lymphoid lineage, include leukemia, acute lymphocytic leukemia, chronic lymphocytic leukemia, acute lymphoblastic leukemia, B-cell lymphoma, T-cell lymphoma, Hodgkins lymphoma, non-Hodgkins lymphoma, hairy cell lymphoma, mantle cell lymphoma, myeloma, and Burkett's lymphoma; hematopoetic tumors of myeloid lineage, including acute and chronic myelogenous leukemias, myelodysplastic syndrome and promyelocytic leukemia; tumors of mesenchymal origin, including fibrosarcoma and rhabdomyosarcoma; tumors of the central and peripheral nervous system, including astrocytoma, neuroblastoma, glioma, and schwannomas; and other tumors, including melanoma, skin (non-melanomal) cancer, mesothelioma (cells), seminoma, teratocarcinoma, osteosarcoma, xenoderoma pigmentosum, keratoctanthoma, thyroid follicular cancer and Kaposi's sarcoma.

In some embodiments, the cancer is selected from the group consisting of anal cancer, biliary tract cancer, bladder cancer, breast cancer, cervical cancer, colon cancer, colorectal cancer (CRC), esophageal cancer, gastrointestinal cancer, glioblastoma, glioma, head and neck cancer (HNSCC), hepatocellular carcinoma (HCC), lung cancer, liver cancer, lymphoma, melanoma, mesothelioma, multiple myeloma, non-small cell lung cancer (NSCLC), ovarian cancer, pancreatic cancer, prostate cancer (e.g. hormone refractory prostate adenocarcinoma), renal cell carcinoma (RCC), salivary cancer, thyroid cancer, and other neoplastic malignancies.

In some embodiments, the cancer is selected from the group consisting of bladder cancer, breast cancer, colorectal cancer (CRC), esophageal cancer, gastrointestinal cancer, hepatocellular carcinoma (HCC), melanoma, non-small cell lung cancer (NSCLC), ovarian cancer, prostate cancer, and renal cell carcinoma (RCC).

In one embodiment, the cancer is advanced RCC. In another embodiment, the cancer is advanced RCC with clear cell component. In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In some embodiments, a combination of a TIGIT antagonist, a PD-1 antagonist, and belzutifan or a pharmaceutically acceptable salt thereof provides enhanced efficacy as compared with existing treatments. For example, TIGIT and PD-L1 are co-expressed in tumors. A combination of a TIGIT antagonist, a PD-1 antagonist, and an HIF-2α inhibitor may provide added benefit over single- or dual-agent checkpoint blockade. In certain embodiments, the tolerability of vibostolimab is a potential added benefit for combination with belzutifan or a pharmaceutically acceptable salt thereof or a pharmaceutically acceptable salt thereof and a PD-1 antagonist.

In certain embodiments, the cancer is metastatic. In some embodiments, the cancer is relapsed. In other embodiments, the cancer is refractory. In yet other embodiments, the cancer is relapsed and refractory.

In certain embodiments, provided herein is a method of treating RCC, comprising administering to a human patient in need thereof.
(a) a TIGIT antagonist;
(b) a PD-1 antagonist; and
(c) belzutifan or a pharmaceutically acceptable salt thereof.

In certain embodiments, the method of treating cancer comprises administering to a human patient in need thereof.
(a) a TIGIT antagonist as disclosed in Section titled TIGIT Antagonists;
(b) a PD-1 antagonist as disclosed in Section titled PD-1 Antagonists; and
(c) an HIF-2α inhibitor as disclosed in Section titled HIF-2α Inhibitors.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

In certain embodiments, the HIF-2α inhibitor is belzutifan or a pharmaceutically acceptable salt thereof.

Thus, in certain embodiments, provided herein is a method for treating cancer, comprising administering to a human patient in need thereof:
(a) a human or humanized anti-human TIGIT monoclonal antibody or antigen binding fragment thereof;
(b) a human or humanized anti-human PD-1 monoclonal antibody or antigen binding fragment thereof; and
(c) an HIF-2α inhibitor (e.g., belzutifan or a pharmaceutically acceptable salt thereof).

In some embodiments, provided herein is a method for treating cancer, comprising administering to a human patient in need thereof:
(a) a human anti-human TIGIT monoclonal antibody or antigen binding fragment thereof;
(b) a human anti-human PD-1 monoclonal antibody or antigen binding fragment thereof; and
(c) an HIF-2α inhibitor (e.g., belzutifan or a pharmaceutically acceptable salt thereof).

In other embodiments, provided herein is a method for treating cancer, comprising administering to a human patient in need thereof:
(a) a humanized anti-human TIGIT monoclonal antibody or antigen binding fragment thereof;
(b) a humanized anti-human PD-1 monoclonal antibody or antigen binding fragment thereof; and
(c) an HIF-2α inhibitor (e.g., belzutifan or a pharmaceutically acceptable salt thereof).

In one embodiment of the methods provided herein, the anti-human PD-1 monoclonal antibody or antigen binding fragment thereof is pembrolizumab.

In another embodiment of the methods provided herein, the anti-human PD-1 monoclonal antibody or antigen binding fragment thereof is nivolumab.

In another embodiment of the methods provided herein, the anti-human PD-1 monoclonal antibody or antigen binding fragment thereof is cemiplimab.

In certain embodiments of the methods provided herein, the anti-TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of the methods provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In certain embodiments, the anti-TIGIT antibody or antigen binding fragment thereof comprises a light chain comprising or consisting of an amino acid sequence as set forth in SEQ ID NO:294 and a heavy chain comprising or consisting of an amino acid sequence as set forth in SEQ ID NO:295.

In one specific embodiment of the methods, kits and uses provided herein, the method for treating cancer comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In one specific embodiment of the methods, kits and uses provided herein, the method for treating cancer comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In one specific embodiment of the methods, kits and uses provided herein, the method for treating cancer comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof.

In one specific embodiment of the methods, kits and uses provided herein, the method for treating RCC comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In one specific embodiment of the methods, kits and uses provided herein, the method for treating RCC comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In one specific embodiment of the methods, kits and uses provided herein, the method for treating RCC comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof.

In another aspect, provided herein are methods of treating von-Hippel Lindau disease using a combination of a TIGIT antagonist, a PD-1 antagonist, and an HIF-2α inhibitor.

In certain embodiments, the TIGIT antagonist is an anti-TIGIT antibody or antigen binding fragment thereof. In some embodiments, the PD-1 antagonist is an anti-PD-1 antibody or antigen binding fragment thereof.

In certain embodiments, the method of treating von-Hippel Lindau disease comprises administering to a human patient in need thereof:
(a) a TIGIT antagonist;
(b) a PD-1 antagonist; and
(c) belzutifan or a pharmaceutically acceptable salt thereof.

In some embodiments, a combination of a TIGIT antagonist, a PD-1 antagonist, and belzutifan or a pharmaceutically acceptable salt thereof provides enhanced efficacy as compared with existing treatments. In certain embodiments, the tolerability of vibostolimab is a potential added benefit for combination with belzutifan or a pharmaceutically acceptable salt thereof or a pharmaceutically acceptable salt thereof and a PD-1 antagonist.

In certain embodiments, provided herein is a method of treating von-Hippel Lindau disease, comprising administering to a human patient in need thereof:
(a) a TIGIT antagonist;
(b) a PD-1 antagonist; and
(c) belzutifan or a pharmaceutically acceptable salt thereof.

In certain embodiments, the method of treating von-Hippel Lindau disease comprises administering to a human patient in need thereof:
(a) a TIGIT antagonist as disclosed in Section titled TIGIT Antagonists;
(b) a PD-1 antagonist as disclosed in Section titled PD-1 Antagonists; and
(c) an HIF-2α inhibitor as disclosed in Section titled HIF-2α Inhibitors.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

In certain embodiments, the PD-1 antagonist is an anti-human PD-L1 monoclonal antibody or antigen binding fragment thereof. In some embodiments, the anti-human PD-L1 monoclonal antibody is a human antibody. In other embodiments, the anti-human PD-L1 monoclonal antibody is a humanized antibody.

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

In certain embodiments, the HIF-2α inhibitor is belzutifan or a pharmaceutically acceptable salt thereof.

Thus, in certain embodiments, provided herein is a method for treating von-Hippel Lindau disease, comprising administering to a human patient in need thereof:
(a) a human or humanized anti-human TIGIT monoclonal antibody or antigen binding fragment thereof;
(b) a human or humanized anti-human PD-1 monoclonal antibody or antigen binding fragment thereof; and
(c) an HIF-2α inhibitor (e.g., belzutifan or a pharmaceutically acceptable salt thereof).

In some embodiments, provided herein is a method for treating von-Hippel Lindau disease, comprising administering to a human patient in need thereof:
(a) a human anti-human TIGIT monoclonal antibody or antigen binding fragment thereof;
(b) a human anti-human PD-1 monoclonal antibody or antigen binding fragment thereof; and
(c) an HIF-2α inhibitor (e.g., belzutifan or a pharmaceutically acceptable salt thereof).

In other embodiments, provided herein is a method for treating von-Hippel Lindau disease, comprising administering to a human patient in need thereof:
(a) a humanized anti-human TIGIT monoclonal antibody or antigen binding fragment thereof;
(b) a humanized anti-human PD-1 monoclonal antibody or antigen binding fragment thereof; and
(c) an HIF-2α inhibitor (e.g., belzutifan or a pharmaceutically acceptable salt thereof).

In one embodiment of the methods provided herein, the anti-human PD-1 monoclonal antibody or antigen binding fragment thereof is pembrolizumab.

In another embodiment of the methods provided herein, the anti-human PD-1 monoclonal antibody or antigen binding fragment thereof is nivolumab.

In another embodiment of the methods provided herein, the anti-human PD-1 monoclonal antibody or antigen binding fragment thereof is cemiplimab.

In certain embodiments of the methods provided herein, the anti-TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of the methods provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In certain embodiments, the anti-TIGIT antibody or antigen binding fragment thereof comprises a light chain comprising or consisting of an amino acid sequence as set forth in SEQ ID NO:294 and a heavy chain comprising or consisting of an amino acid sequence as set forth in SEQ ID NO:295.

In one specific embodiment of the methods, kits and uses provided herein, the method for treating von-Hippel Lindau disease comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In one specific embodiment of the methods, kits and uses provided herein, the method for treating von-Hippel Lindau disease comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In one specific embodiment of the methods, kits and uses provided herein, the method for treating von-Hippel Lindau disease comprises administering to a human patient in need thereof:
(a) an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof

### Dosing and Administration

Further provided herein are dosing regimens and routes of administration for treating cancer (e.g., RCC) or von-Hippel Lindau disease using a combination of a TIGIT antagonist (e.g., an anti-TIGIT monoclonal antibody or antigen binding fragment thereof), a PD-1 antagonist (e.g., an anti-PD-1 monoclonal antibody or antigen binding fragment thereof), and an HIF-2α inhibitor (e.g., belzutifan or a pharmaceutically acceptable salt thereof).

The anti-TIGIT antibody (e.g., anti-TIGIT monoclonal antibody) or antigen binding fragment thereof, the anti-PD-1 antibody (e.g., anti-PD-1 monoclonal antibody) or antigen binding fragment thereof, or the HIF-2α inhibitor (e.g., belzutifan or a pharmaceutically acceptable salt thereof) disclosed herein may be administered by doses administered, e.g., daily, 1-7 times per week, weekly, bi-weekly, tri-weekly, every four weeks, every five weeks, every 6 weeks, monthly, bimonthly, quarterly, semiannually, annually, etc. Doses may be administered, e.g., intravenously, subcutaneously, topically, orally, nasally, rectally, intramuscular, intracerebrally, intraspinally, or by inhalation. In certain embodiments, the doses are administered intravenously. In certain embodiments, the doses are administered subcutaneously. In certain embodiments, the doses are administered orally.

In some embodiments, the anti-TIGIT antibody (e.g., anti-TIGIT monoclonal antibody) thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at about 10, about 20, about 50, about 80, about 100, about 200, about 300, about 400, about 500, about 1000 or about 2500 mg/subject.

In some specific methods, the dose of the anti-TIGIT antibody (e.g., anti-TIGIT monoclonal antibody) or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 monoclonal antibody or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 monoclonal antibody or antigen binding fragment thereof is about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg.

In some embodiments, the anti-PD-1 antibody (e.g., anti-PD-1 monoclonal antibody) or antigen binding fragment thereof is administered subcutaneously or intravenously, on a weekly, biweekly, triweekly, every 4 weeks, every 5 weeks, every 6 weeks, every 8 weeks, every 9 weeks, every 10 weeks, every 12 weeks, monthly, bimonthly, or quarterly basis at about 10, about 20, about 50, about 80, about 100, about 200, about-300, about 400, about 500, about 1000 or about 2500 mg/subject.

In some specific methods, the dose of the anti-PD-1 antibody (e.g., anti-PD-1 monoclonal antibody) or antigen binding fragment thereof is from about 0.01 mg/kg to about 50 mg/kg, from about 0.05 mg/kg to about 25 mg/kg, from about 0.1 mg/kg to about 10 mg/kg, from about 0.2 mg/kg to about 9 mg/kg, from about 0.3 mg/kg to about 8 mg/kg, from about 0.4 mg/kg to about 7 mg/kg, from about 0.5 mg/kg to about 6 mg/kg, from about 0.6 mg/kg to about 5 mg/kg, from about 0.7 mg/kg to about 4 mg/kg, from about 0.8 mg/kg to about 3 mg/kg, from about 0.9 mg/kg to about 2 mg/kg, from about 1.0 mg/kg to about 1.5 mg/kg, from about 1.0 mg/kg to about 2.0 mg/kg, from about 1.0 mg/kg to about 3.0 mg/kg, or from about 2.0 mg/kg to about 4.0 mg/kg. In some specific methods, the dose of the anti-PD-1 antibody (e.g., anti-PD-1 monoclonal antibody) or antigen binding fragment thereof is from about 10 mg to about 500 mg, from about 25 mg to about 500 mg, from about 50 mg to about 500 mg, from about 100 mg to about 500 mg, from about 200 mg to about 500 mg, from about 150 mg to about 250 mg, from about 175 mg to about 250 mg, from about 200 mg to about 250 mg, from about 150 mg to about 240 mg, from about 175 mg to about 240 mg, or from about 200 mg to about 240 mg. In some embodiments, the dose of the anti-PD-1 antibody (e.g., anti-PD-1 monoclonal antibody) or antigen binding fragment thereof is about 50 mg, about 75 mg, about 100 mg, about 125 mg, about 150 mg, about 175 mg, about 200 mg, about 225 mg, about 240 mg, about 250 mg, about 300 mg, about 400 mg, or about 500 mg.

In some embodiments of the methods, compositions, kits and uses described herein, the anti-TIGIT antibody (e.g., anti-TIGIT monoclonal antibody) comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered about 100 mg, about 150 mg, about 200 mg, about 240 mg, about 400 mg, about 480 mg, or about 720 mg, or about 2 mg/kg anti-TIGIT antibody (e.g., anti-TIGIT monoclonal antibody), and the anti-TIGIT antibody (e.g., anti-TIGIT monoclonal antibody) is administered once every three or six weeks. In one embodiment, the human patient is administered about 200 mg anti-TIGIT antibody (e.g., anti-TIGIT monoclonal antibody) once every three weeks. In one embodiment, the human patient is administered 240 mg anti-TIGIT antibody (e.g., anti-TIGIT monoclonal antibody) once every three weeks. In one embodiment, the human patient is administered 2 mg/kg anti-TIGIT antibody (e.g., anti-TIGIT monoclonal antibody) once every three weeks. In one embodiment, the human patient is administered 400 mg anti-TIGIT antibody (e.g., anti-TIGIT monoclonal antibody) once every three weeks.

In certain embodiments of the methods described herein, the anti-TIGIT antibody (e.g., anti-TIGIT monoclonal antibody) comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 400 mg anti-TIGIT antibody (e.g., anti-TIGIT monoclonal antibody), and the anti-TIGIT antibody (e.g., anti-TIGIT monoclonal antibody) is administered once every six weeks.

In some embodiments of the methods described herein, the anti-TIGIT antibody (e.g., anti-TIGIT monoclonal antibody) comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 200 mg, 240 mg, 400 mg, 480 mg, 720 mg, or 2 mg/kg anti-TIGIT antibody (e.g., anti-TIGIT monoclonal antibody), and the anti-TIGIT monoclonal antibody is administered once every six weeks. In one embodiment, the human patient is administered 200 mg anti-TIGIT antibody (e.g., anti-TIGIT monoclonal antibody) once every six weeks. In one embodiment, the human patient is administered 240 mg anti-TIGIT antibody (e.g., anti-TIGIT monoclonal antibody) once every six weeks. In one embodiment, the human patient is administered 400 mg anti-TIGIT antibody (e.g., anti-TIGIT monoclonal antibody) once every six weeks. In one embodiment, the human patient is administered about 480 mg anti-TIGIT antibody (e.g., anti-TIGIT monoclonal antibody) once every six weeks. In one embodiment, the human patient is administered 720 mg anti-TIGIT antibody (e.g., anti-TIGIT monoclonal antibody) once every six weeks. In one embodiment, the human patient is administered 2 mg/kg anti-TIGIT antibody (e.g., anti-TIGIT monoclonal antibody) once every six weeks.

In some embodiments of the methods, compositions, kits and uses described herein, the anti-human PD-1 antibody (e.g., anti-PD-1 monoclonal antibody) or antigen binding fragment thereof is pembrolizumab, the human patient is administered about 200 mg, about 240 mg, about 400 mg, about 480 mg, about 720 mg, or about 2 mg/kg pembrolizumab, and pembrolizumab is administered once every three or six weeks. In one embodiment, the human patient is administered about 200 mg pembrolizumab once every three weeks. In one embodiment, the human patient is administered about 240 mg pembrolizumab once every three weeks. In one embodiment, the human patient is administered 2 mg/kg pembrolizumab once every three weeks. In one embodiment, the human patient is administered 400 mg pembrolizumab once every three weeks.

In certain embodiments of the methods, compositions, kits and uses described herein, the anti-human PD-1 monoclonal antibody or antigen binding fragment thereof is pembrolizumab, the human patient is administered 400 mg pembrolizumab, and pembrolizumab is administered once every six weeks.

In some embodiments of the methods, compositions, kits and uses described herein, the anti-human PD-1 monoclonal antibody or antigen binding fragment thereof is pembrolizumab, the human patient is administered about 200 mg, about 240 mg, about 400 mg, about 480 mg, about 720 mg, or about 2 mg/kg pembrolizumab, and pembrolizumab is administered once every six weeks. In one embodiment, the human patient is administered about 200 mg pembrolizumab once every six weeks. In one embodiment, the human patient is administered about 240 mg pembrolizumab once every six weeks. In one embodiment, the human patient is administered about 400 mg pembrolizumab once every six weeks. In one embodiment, the human patient is administered 480 mg pembrolizumab once every six weeks. In one embodiment, the human patient is administered 720 mg pembrolizumab once every six weeks. In one embodiment, the human patient is administered 2 mg/kg pembrolizumab once every six weeks.

In some embodiments of the invention, the anti-TIGIT antibody, or antigen binding fragment thereof, and the anti-PD-1 antibody, or antigen binding fragment thereof, are administered to the patient once every approximately six weeks for 12 weeks or more. In other embodiments, the anti-TIGIT antibody, or antigen binding fragment and the anti-PD-1 antibody, or antigen binding fragment thereof, are administered to the patient once every six weeks for 18 weeks or more, 24 weeks or more, 30 weeks or more, 36 weeks or more, 42 weeks or more, 48 weeks or more, 54 weeks or more, 60 weeks or more, 66 weeks or more, 72 weeks or more, 78 weeks or more, 84 weeks or more, or 90 weeks or more. In one embodiment, the administration occurs on the same day.

In a sub-embodiment, the anti-TIGIT antibody, or antigen binding fragment thereof, and the anti-PD-1 antibody, or antigen binding fragment thereof, are administered on the same day simultaneously (e.g., in a single formulation, a co-formulation or concurrently as separate formulations). In an alternative embodiment, the anti-TIGIT antibody or antigen binding fragment thereof and the anti-PD-1 antibody or antigen binding fragment thereof are administered sequentially on the same day (e.g., as separate formulations), in either order. In one embodiment of same day sequential administration, the anti-TIGIT antibody or antigen binding fragment thereof is administered first. In another embodiment of same day sequential administration, the anti-PD-1 antibody or antigen binding fragment thereof is administered first.

In certain embodiments of the methods, compositions, kits and uses described herein, the anti-human TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered 200 mg anti-human TIGIT antibody, and the anti-human TIGIT antibody is administered once every three weeks. In certain embodiments of the methods, compositions, kits and uses described herein, the anti-human TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110, the human patient is administered about 400 mg anti-human TIGIT antibody, and the anti-human TIGIT antibody is administered once every six weeks.

In certain embodiments of the methods, compositions, kits and uses described herein, the anti-human PD-1 monoclonal antibody or antigen binding fragment thereof is pembrolizumab, the human patient is administered about 200 mg pembrolizumab, and pembrolizumab is administered once every three weeks. In certain embodiments of the methods, compositions, kits and uses described herein, the anti-human PD-1 monoclonal antibody or antigen binding fragment thereof is pembrolizumab, the human patient is administered about 400 mg pembrolizumab, and pembrolizumab is administered once every six weeks.

In other embodiments of the methods, compositions, kits and uses described herein, the anti-human PD-1 monoclonal antibody or antigen binding fragment thereof is nivolumab, the human patient is administered about 240 mg or about 3 mg/kg nivolumab, and nivolumab is administered once every two weeks. In one specific embodiment, the human patient is administered about 240 mg nivolumab once every two weeks. In one specific embodiment, the human patient is administered about 3 mg/kg nivolumab once every two weeks. In other embodiments of the methods, compositions, kits and uses described herein, the anti-human PD-1 monoclonal antibody or antigen binding fragment thereof is nivolumab, the human patient is administered about 480 mg nivolumab, and nivolumab is administered once every four weeks.

In yet other embodiments of the methods, compositions, kits and uses described herein, the anti-human PD-1 monoclonal antibody or antigen binding fragment thereof is cemiplimab, the human patient is administered about 350 mg cemiplimab, and cemiplimab is administered once every three weeks.

In some embodiments, an anti-TIGIT antibody and anti-PD-1 antibody are co-formulated. In one embodiment, a co-formulated product with about 200 mg pembrolizumab or a pembrolizumab variant and 200 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In one embodiment, a co-formulated product with 200 mg pembrolizumab or a pembrolizumab variant and 300 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In one embodiment, a co-formulated product with about 200 mg pembrolizumab or a pembrolizumab variant and about 400 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with about 200 mg of pembrolizumab or a pembrolizumab variant and about 500 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with of about 200 mg pembrolizumab or a pembrolizumab variant and about 600 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion. In another embodiment, a co-formulated product with about 200 mg of pembrolizumab or a pembrolizumab variant and about 700 mg of antibody comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 is used for intravenous infusion.

In some embodiments of the methods, compositions, kits and uses described herein, the HIF-2α inhibitor is belzutifan or a pharmaceutically acceptable salt thereof, the human patient is administered about 10 mg, about 20 mg, about 30 mg, about 40 mg, about 50 mg, about 60 mg, about 70 mg, about 80 mg, about 90 mg, about 100 mg, about 110 mg, about 120 mg, about 130 mg, about 140 mg, or about 150 mg belzutifan or a pharmaceutically acceptable salt thereof once a day.

Thus, in some embodiments of the methods, compositions, kits and uses provided herein, the human patient is administered:
(a) about 100 mg, about 150 mg, about 200 mg, about 250 mg, or about 400 mg, or about 2 mg/kg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 100 mg, about 150 mg, about 200 mg, about 250 mg, or about 400 mg, or about 2 mg/kg pembrolizumab; and
(c) about 40 mg, about 80 mg, or about 120 mg belzutifan or a pharmaceutically acceptable salt thereof,
wherein (a) and (b) are administered once every three or six weeks, and (c) is administered once a day.

In certain embodiments of the methods, compositions, kits and uses provided herein, the human patient is administered:
(a) about 200 mg, about 240 mg, about 400 mg, about 2 mg/kg or about 22 mg/mL of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110; and
(b) about 200 mg, about 240 mg, about 400 mg, about 2 mg/kg or about 22 mg/mL pembrolizumab; and
(c) about 40 mg, about 80 mg, or about 120 mg belzutifan or a pharmaceutically acceptable salt thereof,
wherein (a) and (b) are administered once every three weeks, and (c) is administered once a day.

In certain embodiments of the methods, compositions, kits and uses provided herein, the human patient is administered:
(a) about 200 mg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 120 mg belzutifan or a pharmaceutically acceptable salt thereof,
wherein (a) and (b) are administered once every three weeks and (c) is administered once a day.

In certain embodiments of the methods provided herein, the human patient is administered:
(a) about 200 mg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 200 mg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 200 mg of an anti-TIGIT antibody or antigen binding fragment thereof comprising a heavy chain variable region comprising the amino acid sequence as set forth in SEQ ID NO: 148 and a light chain variable region comprising the amino acid sequence as set forth in SEQ ID NO: 152;
(b) about 200 mg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 200 mg of an anti-TIGIT antibody or antigen binding fragment thereof comprising a light chain comprising or consisting of an amino acid sequence as set forth in SEQ ID NO: 294 and a heavy chain comprising or consisting of an amino acid sequence as set forth in SEQ ID NO: 295;
(b) about 200 mg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 240 mg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 240 mg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 2 mg/kg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 2 mg/kg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 400 mg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) about 400 mg pembrolizumab; and
(c) about 120 mg belzutifan or a pharmaceutically acceptable salt thereof,
wherein (a) and (b) are administered once every three weeks and (c) is administered once a day.

In certain embodiments of the methods, compositions, kits and uses provided herein, the human patient is administered:
(a) about 200 mg of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 once every three weeks;
(b) about 400 mg pembrolizumab once every six weeks; and
(c) about 120 mg belzutifan or a pharmaceutically acceptable salt thereof once a day
wherein (a) and (b) are co-formulated or co-administered.

In certain embodiments of the methods, compositions, kits and uses described herein, a co-formulation of pembrolizumab and of an anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110 are administered by IV infusion. In some embodiments, the co-formulation is administered for about 30 minutes every three weeks. In some embodiments, the co-formulation is administered for from about 25 to about 40 minutes every three weeks.

In some embodiments, at least one of the therapeutic agents (e.g., the anti-TIGIT monoclonal antibody or binding fragment thereof, the anti-PD-1 monoclonal antibody or binding fragment thereof, or belzutifan or a pharmaceutically acceptable salt thereof) in the combination therapy is administered using the same dosage regimen (dose, frequency, and duration of treatment) that is typically employed when the agent is used as monotherapy for treating the same condition. In other embodiments, the patient receives a lower total amount of at least one of the therapeutic agents (e.g., the anti-TIGIT monoclonal antibody or binding fragment thereof, the anti-PD-1 monoclonal antibody or binding fragment thereof, or belzutifan or a pharmaceutically acceptable salt thereof) in the combination therapy than when the agent is used as monotherapy, e.g., smaller doses, less frequent doses, and/or shorter treatment duration.

A combination therapy disclosed herein may be used prior to or following surgery to remove a tumor and may be used prior to, during, or after radiation treatment.

In some embodiments, a combination therapy disclosed herein is administered to a patient who has not previously been treated with a biotherapeutic or chemotherapeutic agent, i.e., is treatment-naïve. In other embodiments, the combination therapy is administered to a patient who failed to achieve a sustained response after prior therapy with the biotherapeutic or chemotherapeutic agent, i.e., is treatment-experienced.

The therapeutic combination disclosed herein may be used in combination with one or more other active agents, including but not limited to, other anti-cancer agents that are used in the prevention, treatment, control, amelioration, or reduction of risk of a particular disease or condition (e.g., cancer). Such other active agents may be administered, by a route and in an amount commonly used therefor, contemporaneously or sequentially with one or more of the therapeutic agents in the combinations disclosed herein.

The one or more additional active agents may be co-administered with the anti-TIGIT monoclonal antibody or antigen binding fragment thereof, the anti-PD-1 monoclonal antibody or antigen binding fragment thereof, or belzutifan or a pharmaceutically acceptable salt thereof. The additional active agent(s) can be administered in a single dosage form with one or more co-administered agent selected from the anti-TIGIT monoclonal antibody or antigen binding fragment thereof, the anti-PD-1 monoclonal antibody or antigen binding fragment thereof, and belzutifan or a pharmaceutically acceptable salt thereof. The additional active agent(s) can also be administered in separate dosage form(s) from the dosage forms containing the anti-TIGIT monoclonal antibody or antigen binding fragment thereof, the anti-PD-1 monoclonal antibody or antigen binding fragment thereof, or belzutifan or a pharmaceutically acceptable salt thereof.

### Pharmaceutical Compositions

In yet another aspect, provided herein are pharmaceutical compositions comprising the therapeutic agents disclosed herein (e.g., a TIGIT antagonist, a PD-1 antagonist, or belzutifan or a pharmaceutically acceptable salt thereof).

In certain embodiments, the pharmaceutical composition further comprises a pharmaceutically acceptable carrier.

The pharmaceutical compositions comprising an anti-human TIGIT antibody (e.g., anti-TIGIT monoclonal antibody) or antigen binding fragment thereof, an anti-human PD-1 antibody (e.g., anti-PD-1 monoclonal antibody) or antigen binding fragment thereof, and belzutifan or a pharmaceutically acceptable salt thereof can be prepared for storage by mixing the antibodies or compounds having the desired degree of purity with optionally physiologically acceptable carriers, excipients, or stabilizers (see, e.g., Remington, Remington's Pharmaceutical Sciences (18^{th} ed. 1980)) in the form of aqueous solutions or lyophilized or other dried forms.

The pharmaceutically acceptable carriers, excipients, or stabilizers are non-toxic to the cell or mammalian being exposed thereto at the dosage and concentrations employed. Often the pharmaceutically acceptable carrier is an aqueous pH buffered solution. Examples of pharmaceutically acceptable carriers include buffers, such as phosphate, citrate, acetate, and other organic acids; antioxidants, such as ascorbic acid; low molecular weight (e.g., fewer than about 10 amino acid residues) polypeptide; proteins, such as serum albumin, gelatin, or immunoglobulin; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. The pharmaceutically acceptable carriers can also refer to a diluent, adjuvate (e.g., Freund's adjuvate (complete or incomplete)), excipient, or vehicle. Such carriers can be sterile liquids, such as water and oils, including those of petroleum, animal, vegetable, or synthetic origin, such as peanut oil, soybean oil, mineral oil, sesame oil, and the like. Water is an exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsions, tablets, pills, capsules, powders, sustained-release formulations, and the like.

### Kits

In still another aspect, provided herein are kits comprising the therapeutic agents disclosed herein (e.g., a TIGIT antagonist, a PD-1 antagonist, and an HIF-2α inhibitor, optionally, chemotherapeutic agents) or pharmaceutical compositions thereof, packaged into suitable packaging material. A kit optionally includes a label or packaging insert that include a description of the components or instructions for use in vitro, in vivo, or ex vivo, of the components therein.

In some embodiments, the kit comprises
(a) a TIGIT antagonist;
(b) a PD-1 antagonist; and
(c) belzutifan or a pharmaceutically acceptable salt thereof.

In certain embodiments, the kit further comprises instructions for administering to a human patient the TIGIT antagonist, the PD-1 antagonist, and belzutifan or a pharmaceutically acceptable salt thereof.

In some embodiments, the TIGIT antagonist is an anti-TIGIT monoclonal antibody or antigen-binding fragment thereof. In some embodiments, the PD-1 antagonist is an anti-PD-1 monoclonal antibody or antigen-binding fragment thereof. In some embodiments, the PD-1 antagonist is an anti-PD-L1 monoclonal antibody or antigen-binding fragment thereof.

In one embodiment, the kit comprises: (a) one or more dosages of an anti-TIGIT antibody (e.g., anti-TIGIT monoclonal antibody) or antigen binding fragment thereof, (b) one or more dosages of an anti-PD-1 antibody (e.g., anti-PD-1 monoclonal antibody or antigen binding fragment thereof; (c) one or more dosages of belzutifan or a pharmaceutically acceptable salt thereof; and (d) instructions for administering to a human patient the anti-human TIGIT antibody (e.g., anti-TIGIT monoclonal antibody) or antigen binding fragment thereof, the anti-human PD-1 antibody (e.g., anti-PD-1 monoclonal antibody) or antigen binding fragment thereof, and belzutifan or a pharmaceutically acceptable salt thereof.

In some embodiments, the anti-PD-1 monoclonal antibody or antigen binding fragment thereof is pembrolizumab. In some embodiments, the anti-PD-1 monoclonal antibody or antigen binding fragment thereof is nivolumab. In some embodiments, the anti-PD-1 monoclonal antibody or antigen binding fragment thereof is cemiplimab.

The dosages for the anti-TIGIT antibody (e.g., anti-TIGIT monoclonal antibody), the anti-PD-1 antibody (e.g., anti-PD-1 monoclonal antibody), or belzutifan or a pharmaceutically acceptable salt thereof described herein can be used in the kits herein. In some embodiments, a kit comprises dosages of each component sufficient for a certain period of treatment (e.g., 3, 6, 12, or 24 weeks, etc.). For example, a kit can comprise one dosage of about 200 mg pembrolizumab, one dosage of about 200 mg anti-TIGIT antibody, and one dosage of about 120 mg belzutifan or a pharmaceutically acceptable salt thereof, which are sufficient for a 3-week treatment. Or, a kit can also comprise one dosage of about 400 mg pembrolizumab, one dosage of about 400 mg anti-TIGIT antibody, and two dosages of about 120 mg belzutifan or a pharmaceutically acceptable salt thereof, which are sufficient for a 6-week treatment.

In some embodiments, the kit comprises means for separately retaining the components, such as a container, divided bottle, or divided foil packet. A kit of this disclosure can be used for administration of different dosage forms, for example, oral and parenteral, for administration of the separate compositions at different dosage intervals, or for titration of the separate compositions against one another.

### Uses of a Therapeutic Combination for Treating Cancer or Von-Hippel Lindau Disease

In still another aspect, provided herein are uses of a therapeutic combination for treating cancer (e.g., RCC) in a human patient, wherein the therapeutic combination comprises:
(a) a TIGIT antagonist;
(b) a PD-1 antagonist; and
(c) an HIF-2α inhibitor (e.g., belzutifan or a pharmaceutically acceptable salt thereof).

In some embodiments, the cancer is selected from the group consisting of bladder cancer, breast cancer, colorectal cancer (CRC), esophageal cancer, gastrointestinal cancer, hepatocellular carcinoma (HCC), melanoma, non-small cell lung cancer (NSCLC), ovarian cancer, prostate cancer, and renal cell carcinoma (RCC).

In certain embodiments, the cancer is metastatic. In some embodiments, the cancer is relapsed. In other embodiments, the cancer is refractory. In yet other embodiments, the cancer is relapsed and refractory.

In some embodiments, the cancer is bladder cancer. In some embodiments, the cancer is breast cancer, or colorectal cancer (CRC). In some embodiments, the cancer is esophageal cancer. In some embodiments, the cancer is gastrointestinal cancer. In some embodiments, the cancer is hepatocellular carcinoma (HCC). In some embodiments, the cancer is melanoma. In some embodiments, the cancer is non-small cell lung cancer (NSCLC). In some embodiments, the cancer is ovarian cancer. In some embodiments, the cancer is prostate cancer. In some embodiments, the cancer is renal cell carcinoma (RCC).

In one embodiment, the cancer is advanced RCC. In another embodiment, the cancer is advanced RCC with clear cell component. In yet another embodiment, the cancer is metastatic RCC. In yet another embodiment, the cancer is relapsed RCC. In still another embodiment, the cancer is refractory RCC. In yet still another embodiment, the cancer is relapsed and refractory RCC.

In one embodiment, provided herein is use of a therapeutic combination for treating RCC cancer in a human patient, wherein the therapeutic combination comprises:
(a) a TIGIT antagonist;
(b) a PD-1 antagonist; and
(c) belzutifan or a pharmaceutically acceptable salt thereof.

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

Thus, in certain embodiments, provided herein is use of a therapeutic combination for treating cancer, wherein the therapeutic combination comprises:
(a) a human or humanized anti-human TIGIT monoclonal antibody or antigen binding fragment thereof,
(b) a human or humanized anti-human PD-1 monoclonal antibody or antigen binding fragment thereof, and
(c) belzutifan or a pharmaceutically acceptable salt thereof.

In some embodiments, provided herein is use of a therapeutic combination for treating cancer, wherein the therapeutic combination comprises:
(a) a human anti-human TIGIT monoclonal antibody or antigen binding fragment thereof,
(b) a human anti-human PD-1 monoclonal antibody or antigen binding fragment thereof, and
(c) belzutifan or a pharmaceutically acceptable salt thereof.

In some embodiments of the uses provided herein, the anti-PD-1 monoclonal antibody or antigen binding fragment thereof is pembrolizumab. In some embodiments of the uses provided herein, the anti-PD-1 monoclonal antibody or antigen binding fragment thereof is nivolumab. In some embodiments of the uses provided herein, the anti-PD-1 monoclonal antibody or antigen binding fragment thereof is cemiplimab.

In certain embodiments of various uses provided herein, the anti-TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of various uses provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In other embodiments of various uses provided herein, the anti-human TIGIT monoclonal antibody or antigen binding fragment thereof comprises a light chain comprising or consisting of an amino acid sequence as set forth in SEQ ID NO:294 and a heavy chain comprising or consisting of an amino acid sequence as set forth in SEQ ID NO:295.

Thus, in one specific embodiment, provided herein is use of a therapeutic combination for treating cancer, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In some embodiments, provided herein is use of a therapeutic combination for treating cancer, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In some embodiments, provided herein is use of a therapeutic combination for treating cancer, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof.

In some embodiments, provided herein is use of a therapeutic combination for treating RCC, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In some embodiments, provided herein is use of a therapeutic combination for treating RCC, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In some embodiments, provided herein is use of a therapeutic combination for treating RCC, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof.

In still another aspect, provided herein are uses of a therapeutic combination for treating von-Hippel Lindau disease in a human patient, wherein the therapeutic combination comprises:
(d) a TIGIT antagonist;
(e) a PD-1 antagonist; and
(f) an HIF-2α inhibitor (e.g., belzutifan or a pharmaceutically acceptable salt thereof).

In certain embodiments, the TIGIT antagonist is an anti-human TIGIT monoclonal antibody or antigen binding fragment thereof. In some embodiments, the anti-human TIGIT monoclonal antibody is a human antibody. In other embodiments, the anti-human TIGIT monoclonal antibody is a humanized antibody.

In certain embodiments, the PD-1 antagonist is an anti-human PD-1 monoclonal antibody or antigen binding fragment thereof. In some embodiments, the anti-human PD-1 monoclonal antibody is a human antibody. In other embodiments, the anti-human PD-1 monoclonal antibody is a humanized antibody.

Thus, in certain embodiments, provided herein is use of a therapeutic combination for treating von-Hippel Lindau disease, wherein the therapeutic combination comprises:
(a) a human or humanized anti-human TIGIT monoclonal antibody or antigen binding fragment thereof;
(b) a human or humanized anti-human PD-1 monoclonal antibody or antigen binding fragment thereof; and
(c) belzutifan or a pharmaceutically acceptable salt thereof.

In some embodiments, provided herein is use of a therapeutic combination for treating von-Hippel Lindau disease, wherein the therapeutic combination comprises:
(a) a human anti-human TIGIT monoclonal antibody or antigen binding fragment thereof;
(b) a human anti-human PD-1 monoclonal antibody or antigen binding fragment thereof; and
(c) belzutifan or a pharmaceutically acceptable salt thereof.

In some embodiments of the uses provided herein, the anti-PD-1 monoclonal antibody or antigen binding fragment thereof is pembrolizumab. In some embodiments of the uses provided herein, the anti-PD-1 monoclonal antibody or antigen binding fragment thereof is nivolumab. In some embodiments of the uses provided herein, the anti-PD-1 monoclonal antibody or antigen binding fragment thereof is cemiplimab.

In certain embodiments of various uses provided herein, the anti-TIGIT antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

In some embodiments of various uses provided herein, the anti-TIGIT antibody or antigen binding fragment thereof comprises a heavy chain variable region comprising the amino acid sequence as set forth in SEQ ID NO: 148 and a light chain variable region comprising the amino acid sequence as set forth in SEQ ID NO: 152.

In other embodiments of various uses provided herein, the anti-human TIGIT monoclonal antibody or antigen binding fragment thereof comprises a light chain comprising or consisting of an amino acid sequence as set forth in SEQ ID NO:294 and a heavy chain comprising or consisting of an amino acid sequence as set forth in SEQ ID NO:295.

Thus, in one specific embodiment, provided herein is use of a therapeutic combination for treating von-Hippel Lindau disease, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) pembrolizumab; and
(c) belzutifan or a pharmaceutically acceptable salt thereof.

In some embodiments, provided herein is use of a therapeutic combination for treating von-Hippel Lindau disease, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) nivolumab; and
(c) belzutifan or a pharmaceutically acceptable salt thereof.

In some embodiments, provided herein is use of a therapeutic combination for treating cancer, wherein the therapeutic combination comprises:
(a) anti-TIGIT antibody or antigen binding fragment thereof comprising three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(b) cemiplimab; and
(c) belzutifan or a pharmaceutically acceptable salt thereof.

A number of embodiments of the invention have been described. It will be understood that various modifications may be made without departing from the spirit and scope of the invention. It will be further understood that each embodiment may be combined with one or more other embodiments, to the extent that such a combination is consistent with the description of the embodiments.

### General Methods

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, NY; Sambrook and Russell (2001) Molecular Cloning, 3^{rd} ed., Cold Spring Harbor Laboratory Press, Cold Spring Harbor, NY; Wu (1993) Recombinant DNA, Vol. 217, Academic Press, San Diego, CA). Standard methods also appear in Ausbel, et al. (2001) Current Protocols in Molecular Biology, Vols. 1-4, John Wiley and Sons, Inc. New York, NY, which describes cloning in bacterial cells and DNA mutagenesis (Vol. 1), cloning in mammalian cells and yeast (Vol. 2), glycoconjugates and protein expression (Vol. 3), and bioinformatics (Vol. 4).

Methods for protein purification including immunoprecipitation, chromatography, electrophoresis, centrifugation, and crystallization are described (Coligan, et al. (2000) Current Protocols in Protein Science, Vol. 1, John Wiley and Sons, Inc., New York). Chemical analysis, chemical modification, post-translational modification, production of fusion proteins, glycosylation of proteins are described (see, e.g., Coligan, et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, NY, pp. 16.0.5-16.22.17; Sigma-Aldrich, Co. (2001) Products for Life Science Research, St. Louis, MO; pp. 45-89; Amersham Pharmacia Biotech (2001) BioDirectory, Piscataway, N.J., pp. 384-391). Production, purification, and fragmentation of polyclonal and monoclonal antibodies are described (Coligan, et al. (2001) Current Protocols in Immunology, Vol. 1, John Wiley and Sons, Inc., New York; Harlow and Lane (1999) Using Antibodies, Cold Spring Harbor Laboratory Press, Cold Spring Harbor, NY; Harlow and Lane, supra). Standard techniques for characterizing ligand/receptor interactions are available (see, e.g., Coligan, et al. (2001) Current Protocols in Immunology, Vol. 4, John Wiley, Inc., New York).

Monoclonal, polyclonal, and humanized antibodies can be prepared (see, e.g., Sheperd and Dean (eds.) (2000) Monoclonal Antibodies, Oxford Univ. Press, New York, NY; Kontermann and Dubel (eds.) (2001) Antibody Engineering, Springer-Verlag, New York; Harlow and Lane (1988) Antibodies A Laboratory Manual, Cold Spring Harbor Laboratory Press, Cold Spring Harbor, NY, pp. 139-243; Carpenter, et al. (2000) J. Immunol. 165:6205; He, et al. (1998) J. Immunol. 160:1029; Tang et al. (1999) J. Biol. Chem. 274:27371-27378; Baca et al. (1997) J. Biol. Chem. 272:10678-10684; Chothia et al. (1989) Nature 342:877-883; Foote and Winter (1992) J. Mol. Biol. 224:487-499; U.S. Pat. No. 6,329,511).

An alternative to humanization is to use human antibody libraries displayed on phage or human antibody libraries in transgenic mice (Vaughan et al. (1996) Nature Biotechnol. 14:309-314; Barbas (1995) Nature Medicine 1:837-839; Mendez et al. (1997) Nature Genetics 15:146-156; Hoogenboom and Chames (2000) Immunol. Today 21:371-377; Barbas et al. (2001) Phage Display: A Laboratory Manual, Cold Spring Harbor Laboratory Press, Cold Spring Harbor, New York; Kay et al. (1996) Phage Display of Peptides and Proteins: A Laboratory Manual, Academic Press, San Diego, CA; de Bruin et al. (1999) Nature Biotechnol. 17:397-399).

Purification of antigen is not necessary for the generation of antibodies. Animals can be immunized with cells bearing the antigen of interest. Splenocytes can then be isolated from the immunized animals, and the splenocytes can fused with a myeloma cell line to produce a hybridoma (see, e.g., Meyaard et al. (1997) Immunity 7:283-290; Wright et al. (2000) Immunity 13:233-242; Preston et al., supra; Kaithamana et al. (1999) J. Immunol. 163:5157-5164).

Antibodies can be conjugated, e.g., to small drug molecules, enzymes, liposomes, polyethylene glycol (PEG). Antibodies are useful for therapeutic, diagnostic, kit or other purposes, and include antibodies coupled, e.g., to dyes, radioisotopes, enzymes, or metals, e.g., colloidal gold (see, e.g., Le Doussal et al. (1991) J. Immunol. 146:169-175; Gibellini et al. (1998) J. Immunol. 160:3891-3898; Hsing and Bishop (1999) J. Immunol. 162:2804-2811; Everts et al. (2002) J. Immunol. 168:883-889).

Methods for flow cytometry, including fluorescence activated cell sorting (FACS), are available (see, e.g., Owens, et al. (1994) Flow Cytometry Principles for Clinical Laboratory Practice, John Wiley and Sons, Hoboken, NJ; Givan (2001) Flow Cytometry, 2^{nd} ed.; Wiley-Liss, Hoboken, NJ; Shapiro (2003) Practical Flow Cytometry, John Wiley and Sons, Hoboken, NJ). Fluorescent reagents suitable for modifying nucleic acids, including nucleic acid primers and probes, polypeptides, and antibodies, for use, e.g., as diagnostic reagents, are available (Molecular Probesy (2003) Catalogue, Molecular Probes, Inc., Eugene, OR; Sigma-Aldrich (2003) Catalogue, St. Louis, MO).

Standard methods of histology of the immune system are described (see, e.g., Muller-Harmelink (ed.) (1986) Human Thymus: Histopathology and Pathology, Springer Verlag, New York, NY; Hiatt, et al. (2000) Color Atlas of Histology, Lippincott, Williams, and Wilkins, Phila, PA; Louis, et al. (2002) Basic Histology: Text and Atlas, McGraw-Hill, New York, NY).

Software packages and databases for determining, e.g., antigenic fragments, leader sequences, protein folding, functional domains, glycosylation sites, and sequence alignments, are available (see, e.g., GenBank, Vector NTI® Suite (Informax, Inc, Bethesda, MD); GCG Wisconsin Package (Accelrys, Inc., San Diego, CA); DeCypher® (TimeLogic Corp., Crystal Bay, Nevada); Menne, et al. (2000) Bioinformatics 16: 741-742; Menne, et al. (2000) Bioinformatics Applications Note 16:741-742; Wren, et al. (2002) Comput. Methods Programs Biomed. 68:177-181; von Heijne (1983) Eur. J. Biochem. 133:17-21; von Heijne (1986) Nucleic Acids Res. 14:4683-4690).

### Analytical Methods

Analytical methods suitable for evaluating the product stability include size exclusion chromatography (SEC), dynamic light scattering test (DLS), differential scanning calorimetery (DSC), iso-asp quantification, potency, UV at 340 nm, UV spectroscopy, and Fourier-transform infrared spectroscopy (FTIR). SEC (J. Pharm. Scien., 83:1645-1650, (1994); Pharm. Res., 11:485 (1994); J. Pharm. Bio. Anal., 15:1928 (1997); J. Pharm. Bio. Anal., 14:1133-1140 (1986)) measures percent monomer in the product and gives information of the amount of soluble aggregates. DSC (Pharm. Res., 15:200 (1998); Pharm. Res., 9:109 (1982)) gives information of protein denaturation temperature and glass transition temperature. DLS (American Lab., November (1991)) measures mean diffusion coefficient, and gives information of the amount of soluble and insoluble aggregates. UV at 340 nm measures scattered light intensity at 340 nm and gives information about the amounts of soluble and insoluble aggregates. UV spectroscopy measures absorbance at 278 nm and gives information of protein concentration. FTIR (Eur. J. Pharm. Biopharm., 45:231 (1998); Pharm. Res., 12:1250 (1995); J. Pharm. Scien., 85:1290 (1996); J. Pharm. Scien., 87:1069 (1998)) measures IR spectrum in the amide one region, and gives information of protein secondary structure.

The iso-asp content in the samples is measured using the Isoquant Isoaspartate Detection System (Promega). The kit uses the enzyme Protein Isoaspartyl Methyltransferase (PIMT) to specifically detect the presence of isoaspartic acid residues in a target protein. PIMT catalyzes the transfer of a methyl group from S-adenosyl-L-methionine to isoaspartic acid at the .alpha.-carboxyl position, generating S-adenosyl-L-homocysteine (SAH) in the process. This is a relatively small molecule, and can usually be isolated and quantitated by reverse phase HPLC using the SAH HPLC standards provided in the kit.

The potency or bioidentity of an antibody can be measured by its ability to bind to its antigen. The specific binding of an antibody to its antigen can be quantitated by any method known to those skilled in the art, for example, an immunoassay, such as ELISA (enzyme-linked immunosorbant assay).

All publications mentioned herein are incorporated by reference for the purpose of describing and disclosing methodologies and materials that might be used in connection with the present invention.

Having described different embodiments of the invention herein with reference to the accompanying drawings, it is to be understood that the invention is not limited to those precise embodiments, and that various changes and modifications may be effected therein by one skilled in the art without departing from the scope or spirit of the invention as defined in the appended claims.

### EXAMPLES

The examples in this section are offered by way of illustration, and not by way of limitation.

### Example 1: Clinical Trial of Administering a Combination of an Anti-TIGIT Antibody and an Anti-PD-1 Antibody with Belzutifan or a Pharmaceutically Acceptable Salt Thereof

This study is a Phase 1b/2, rolling arm, multicenter, open-label adaptive design study an anti-TIGIT antagonist and an anti-PD-1 antagonist with belzutifan or a pharmaceutically acceptable salt thereof for the treatment of advanced RCC. Preliminary efficacy in this protocol will be evaluated using objective response rate (ORR) per Response Evaluation Criteria in Solid Tumors (RECIST) 1.1, by Blinded Independent Central Review (BICR). This Example will include participants with First-Line (1L) advanced Clear cell RCC (ccRCC). Among approximately 90 participants, 10 patients will be assigned for a Safety Lead-in Phase and 80 patients will be assigned to an Efficacy Phase. After the combination (i.e., (i) Composition A (i.e., co-formulation of pembrolizumab/vibostolimab; vibostolimab comprising heavy chain of SEQ ID NO: 295 and light chain of SEQ ID NO: 294) and (ii) belzutifan) has demonstrated a tolerable safety profile via the Safety Lead-in Phase, participants will be enrolled in the Efficacy Phase.

### Dosing

The maximum dose/exposure allowed in this study is 200 mg vibostolimab/200 mg pembrolizumab for up to 2 years (35 treatment cycles) of initial treatment/first course. The planned starting dose of belzutifan is 120 mg once daily. The actual starting dose of belzutifan may be less if a lower dose was selected during the Safety Lead-in Phase of this study (or if a lower dose is recommended based on other available safety data).

Dose reduction levels of belzutifan should be as follows:
If previous daily dose 120 mg, resume at 80 mg/day
If previous daily dose 80 mg, resume at 40 mg/day
If previous daily dose 40 mg, discontinue treatment

Guidelines for dose modification for belzutifan are listed in Table 9.

The following should be considered in decisions regarding dose modifications:
As a general approach, all Adverse Events (AEs) should be managed with supportive care at the earliest signs of toxicity considered related to belzutifan treatment. Should this be ineffective, dose reductions or interruptions should be considered to prevent worsening of toxicity.
Dose reductions and/or interruptions, at any time while on study, should be implemented for unacceptable toxicity.
Dose modifications or interruptions may also occur in the setting of lower grade toxicity than defined in Table 9, if the investigator feels it is in the interest of a participant's safety and will optimize drug tolerability.
Interruption of belzutifan treatment for AEs may occur at any time per investigator discretion. An interruption of study intervention for more than 28 days will require Sponsor approval before treatment can be resumed.
Re-escalation to the next higher dose level may be permitted after Sponsor consultation for a participant that has resumed belzutifan at a given dose level for at least 28 days and original toxicity has not reappeared. However, re-escalation of belzutifan is not permitted for events of Grade 3 symptomatic hypoxia.

The AE profile of belzutifan indicates that anemia and hypoxia have been associated with belzutifan treatment.

TABLE 9
Dose Modification Guidelines for Belzutifan-related Toxicities

| Belzutifan-Related Toxicity | | Action |
|---|---|---|
| Anemia | Grade 4 anemia | 1^{st} episode: Hold belzutifan. Once toxicity has |
| | | resolved to ≤Grade 2 or baseline, dose reduce |
| | | belzutifan by one level |
| | | 2^{nd} episode: Permanently discontinue belzutifan |
| Neutropenia/Febrile | Grade 3 febrile neutropenia | 1^{st} and 2^{nd} episodes: Hold belzutifan. Once |
| neutropenia | (ANC <1000/mm³ with a single | toxicity has resolved to ≤Grade 2 or baseline, |
| | temperature of >38.3° C[101° F.] | dose reduce belzutifan by one level |
| | or a sustained temperature of ≥38° | 3^{rd} episode: Permanently discontinue belzutifan |
| | C. [100.4° F.] for more than | |
| | one hour), or | |
| | Grade 4 neutropenia for >5 days | |
| | Grade 4 febrile neutropenia | Permanently discontinue belzutifan |
| Thrombocytopenia | Grade 3 thrombocytopenia with | 1^{st} and 2^{nd} episodes: Hold belzutifan. Once |
| | bleeding | toxicity has resolved to ≤Grade 2 or baseline, |
| | | dose reduce belzutifan by one level |
| | | 3^{rd} episode: Permanently discontinue belzutifan |
| | Grade 4 thrombocytopenia | 1^{st} episode: Hold belzutifan. Once toxicity has |
| | | resolved to ≤Grade 2 or baseline, dose reduce |
| | | belzutifan by one level |
| | | 2^{nd} episode: Permanently discontinue belzutifan |
| Hypoxia | Grade 3 dyspnea | 1^{st} and 2^{nd} episodes: Hold belzutifan. Once |
| Or | Grade 3 hypoxia (asymptomatic), | toxicity has resolved to ≤Grade 2 or baseline, |
| Dyspnea | belzutifan may be continued at | dose reduce belzutifan by one level |
| | the discretion of the Investigator | 3^{rd} episode: Permanently discontinue belzutifan |
| | Grade 3 hypoxia (symptomatic) | |
| | Grade 4 dyspnea | Permanently Discontinue belzutifan |
| | Grade 4 hypoxia | |
| Gastrointestinal | Grade 3 or 4 nausea, vomiting, or | 1^{st} and 2^{nd} episodes: Hold belzutifan. Once |
| | diarrhea if persistent for >48 | toxicity has resolved to ≤Grade 2 or baseline, |
| | hours despite optimal | dose reduce belzutifan by one level |
| | antiemetic or antidiarrheal | 3^{rd} episode: Permanently discontinue belzutifan |
| | therapy | |
| Hepatic | Grade 3 increase in AST and/or | 1^{st} episode: Hold belzutifan. Once toxicity has |
| | ALT levels, if confirmed on | resolved to ≤Grade 2 or baseline, dose reduce |
| | repeat testing within 48 hours | belzutifan by one level |
| | | 2^{nd} episode: Permanently discontinue belzutifan |
| | Grade 4 increase in AST and/or | Permanently discontinue belzutifan |
| | ALT levels, if confirmed on | |
| | repeat testing within 48 hours, or | |
| | Grade 3 or 4 increase in AST | |
| | and/or ALT levels if | |
| | accompanied by a Grade 2 | |
| | increase in bilirubin level and an | |
| | alkaline phosphatase level <2 × | |
| | ULN | |
| Cardiovascular, | Grade 3 or 4 cardiovascular, | Permanently discontinue belzutifan |
| vascular, or | vascular or thrombotic events | |
| thrombotic | | |
| Other Nonlaboratory | Any other nonlaboratory Grade 3 | 1^{st} and 2^{nd} episodes: Hold belzutifan. Once |
| Toxicities | toxicity | toxicity has resolved to ≤Grade 2 or baseline, |
| | | dose reduce belzutifan by one level |
| | | 3^{rd} episode: Permanently discontinue belzutifan |
| | Any other nonlaboratory Grade 4 | Permanently discontinue belzutifan |
| | toxicity | |
| Other Laboratory | Grade 3 or 4 laboratory toxicity | Permanently discontinue belzutifan |
| Toxicities | that does not resolve within 48 | |
| | hours and is considered clinically | |
| | significant by the investigator | |

Abbreviations: ALT = alanine aminotransferase; ANC = absolute neutrophil count; AST = aspartate aminotransferase; ULN = upper limit of normal.
Re-escalation to the next higher dose level may be permitted after Sponsor consultation for a participant that has resumed belzutifan at a given dose level for at least 28 days and original toxicity has not reappeared.
Re-escalation of belzutifan will not be permitted for events of Grade 3 symptomatic hypoxia.
If Action is “dose reduce by one dose level” and participant is already at dose level −2 when a toxicity occurred, belzutifan should be permanently discontinued.

AEs associated with Composition A may represent an immune-related response. These irAEs may occur shortly after the first dose or several months after the last dose and may affect more than one body system simultaneously. Therefore, early recognition and initiation of treatment is critical to reduce complications. Based on existing clinical study data, most immune-related AEs (irAEs) were reversible and could be managed with interruptions of Composition A and/or other supportive care. For suspected irAEs, ensure adequate evaluation to confirm etiology or exclude other causes. Additional procedures or tests such as bronchoscopy, endoscopy, skin biopsy may be included as part of the evaluation.

When study interventions are administered in combination, attribution of an adverse event to a single component is likely to be difficult. Therefore, while the investigator may attribute a toxicity event to Composition A must be held according to the criteria in the Dose Modification and Toxicity Management Guidelines for Immune-Related Adverse Events in Table 10.

When study interventions are administered in combination and if the AE is considered immune-related, Composition A should be held according to recommended Dose Modification criteria. If the toxicity does not resolve or the criteria for resuming treatment are not met, the participant must be discontinued from Composition A. If the toxicities do resolve and conditions are aligned with what is defined in the Dose Modification and Toxicity Management Guidelines for irAEs, Composition A may be restarted at the discretion of the investigator. Dose modifications in response to treatment-related AEs are permitted to keep the participant on study medication, when appropriate.

AEs associated with Composition A exposure may represent an immune-related response. These irAEs may occur shortly after the first dose or several months after the last dose of Composition A and may affect more than one body system simultaneously. Therefore, early recognition and initiation of treatment is critical to reduce complications. Based on existing clinical study data, most irAEs were reversible and could be managed with interruptions of immune-related AEs and/or other supportive care. For suspected irAEs, ensure adequate evaluation to confirm etiology or exclude other causes. Additional procedures or tests such as bronchoscopy, endoscopy, skin biopsy may be included as part of the evaluation.

TABLE 10
Dose Modification and Toxicity Management Guidelines for Immune-
related Adverse Events Associated with Composition A
General instructions:

| | |
|---|---|
| 1. | Severe and life-threatening irAEs should be treated with IV corticosteroids followed by oral |
| | steroids. Other immunosuppressive treatment should begin if the irAEs are not controlled by |
| | corticosteroids. |
| 2. | Pembrolizumab monotherapy, coformulations or IO combinations must be permanently discontinued |
| | if the irAE does not resolve or the corticosteroid dose is not ≤10 mg/day within 12 weeks of |
| | the last treatment. |
| 3. | The corticosteroid taper should begin when the irAE is ≤Grade 1 and continue at least 4 weeks. |
| 4. | If pembrolizumab monotherapy, coformulations or IO combinations have been withheld, treatment |
| | may resume after the irAE decreased to ≤Grade 1 after corticosteroid taper. |

| | Toxicity Grade | Action With | Corticosteroid and/or | |
|---|---|---|---|---|
| irAEs | (CTCAE v5.0) | Composition A | Other Therapies | Monitoring and Follow-up |
| Pneumonitis | Grade 2 | Withhold | Administer | Monitor participants for signs |
| | Recurrent | Permanently | corticosteroids (initial | and symptoms of pneumonitis |
| | Grade 2, | discontinue | dose of 1 to 2 mg/kg | Evaluate participants with |
| | Grade 3 or 4 | | prednisone or | suspected pneumonitis with |
| | | | equivalent) followed | radiographic imaging and initiate |
| | | | by taper | corticosteroid treatment |
| | | | Add prophylactic | |
| | | | antibiotics for | |
| | | | opportunistic | |
| | | | infections | |
| Diarrhea/Colitis | Grade 2 or 3 | Withhold | Administer | Monitor participants for signs |
| | Recurrent | Permanently | corticosteroids (initial | and symptoms of enterocolitis |
| | Grade 3 or | discontinue | dose of 1 to 2 mg/kg | (ie, diarrhea, abdominal pain, |
| | Grade 4 | | prednisone or | blood or mucus in stool with or |
| | | | equivalent) followed | without fever) and of bowel |
| | | | by taper | perforation (ie, peritoneal signs |
| | | | | and ileus) |
| | | | | Participants with ≥Grade 2 |
| | | | | diarrhea suspecting colitis |
| | | | | should consider GI consultation |
| | | | | and performing endoscopy to |
| | | | | rule out colitis |
| | | | | Participants with diarrhea/colitis |
| | | | | should be advised to drink |
| | | | | liberal quantities of clear fluids. |
| | | | | If sufficient oral fluid intake is |
| | | | | not feasible, fluid and |
| | | | | electrolytes should be substituted |
| | | | | via IV infusion |
| AST or ALT | Grade 2 ^{a} | Withhold | Administer | Monitor with liver function tests |
| Elevation or | | | corticosteroids (initial | (consider weekly or more |
| Increased | | | dose of 0.5 to 1 mg/kg | frequently until liver enzyme |
| Bilirubin | | | prednisone or | value returned to baseline or is |
| | | | equivalent) followed | stable) |
| | | | by taper | |
| | Grade 3 ^{b} or 4 ^{c} | Permanently | Administer | |
| | | discontinue | corticosteroids (initial | |
| | | | dose of 1 to 2 mg/kg | |
| | | | prednisone or | |
| | | | equivalent) followed | |
| | | | by taper | |
| T1DM or | New onset | Withhold ^{d} | Initiate insulin | Monitor participants for |
| Hyperglycemia | T1DM or | | replacement therapy | hyperglycemia or other signs |
| | Grade 3 or 4 | | for participants with | and symptoms of diabetes |
| | hyperglycemia | | T1DM | |
| | associated with | | Administer | |
| | evidence of | | antihyperglycemic in | |
| | b-cell failure | | participants with | |
| | | | hyperglycemia | |
| Hypophysitis | Grade 2 | Withhold | Administer | Monitor for signs and symptoms |
| | Grade 3 or 4 | Withhold or | corticosteroids and | of hypophysitis (including |
| | | permanently | initiate hormonal | hypopituitarism and adrenal |
| | | discontinue ^{d} | replacements as | insufficiency) |
| | | | clinically indicated | |
| Hyperthyroidism | Grade 2 | Continue | Treat with nonselective | Monitor for signs and symptoms |
| | Grade 3 or 4 | Withhold or | beta-blockers (e.g., | of thyroid disorders |
| | | permanently | propranolol) or | |
| | | discontinue ^{d} | thionamides as | |
| | | | appropriate | |
| Hypothyroidism | Grade 2, 3 or 4 | Continue | Initiate thyroid | Monitor for signs and symptoms |
| | | | replacement hormones | of thyroid disorders |
| | | | (e.g., levothyroxine or | |
| | | | liothyronine) per | |
| | | | standard of care | |
| Nephritis: | Grade 2 | Withhold | Administer | Monitor changes of renal |
| grading | Grade 3 or 4 | Permanently | corticosteroids | function |
| according to | | discontinue | (prednisone 1 to | |
| increased | | | 2 mg/kg or equivalent) | |
| creatinine or | | | followed by taper | |
| acute kidney | | | | |
| injury | | | | |
| Neurological | Grade 2 | Withhold | Based on severity of | Ensure adequate evaluation to |
| Toxicities | Grade 3 or 4 | Permanently | AE administer | confirm etiology and/or exclude |
| | | discontinue | corticosteroids | other causes |
| Myocarditis | Grade 1 | Withhold | Based on severity of | Ensure adequate evaluation to |
| | Grade 2, 3 or 4 | Permanently | AE administer | confirm etiology and/or exclude |
| | | discontinue | corticosteroids | other causes |
| Exfoliative | Suspected SJS, | Withhold | Based on severity of | Ensure adequate evaluation to |
| Dermatologic | TEN, or | | AE administer | confirm etiology or exclude |
| Conditions | DRESS | | corticosteroids | other causes |
| | Confirmed SJS, | Permanently | | |
| | TEN, or | discontinue | | |
| | DRESS | | | |
| All Other | Persistent | Withhold | Based on severity of | Ensure adequate evaluation to |
| irAEs | Grade 2 | | AE administer | confirm etiology or exclude |
| | Grade 3 | Withhold or | corticosteroids | other causes |
| | | discontinue | | |
| | | based on the | | |
| | | event ^{e} | | |
| | Recurrent | Permanently | | |
| | Grade 3 or | discontinue | | |
| | Grade 4 | | | |

AE(s) = adverse event(s); ALT = alanine aminotransferase; AST = aspartate aminotransferase; CTCAE = Common Terminology Criteria for Adverse Events; DRESS = Drug Rash with Eosinophilia and Systemic Symptom; GI = gastrointestinal; IO = immuno-oncology; ir = immune related; IV = intravenous; SJS = Stevens-Johnson Syndrome; T1DM = type 1 diabetes mellitus; TEN = Toxic Epidermal Necrolysis; ULN = upper limit of normal.
Note:
Non-irAE will be managed as appropriate, following clinical practice recommendations.
^{a} AST/ALT: >3.0 to5.0 × ULN if baseline normal; >3.0 to 5.0 × baseline, if baseline abnormal; bilirubin: >1.5 to 3.0 × ULN if baseline normal; >1.5 to 3.0 × baseline if baseline abnormal
^{b} AST/ALT: >5.0 to 20.0 × ULN, if baseline normal; >5.0 to 20.0 × baseline, if baseline abnormal; bilirubin: >3.0 to 10.0 × ULN if baseline normal; >3.0 to 10.0 × baseline if baseline abnormal
^{c} AST/ALT: >20.0 × ULN, if baseline normal; >20.0 × baseline, if baseline abnormal; bilirubin: >10.0 × ULN if baseline normal; >10.0 × baseline if baseline abnormal
^{d} The decision to withhold or permanently discontinue pembrolizumab monotherapy, coformulations or IO combinations is at the discretion of the investigator or treating physician. If control achieved or ≤Grade 2, pembrolizumab monotherapy, coformulations or IO combinations may be resumed.
^{e} Events that require discontinuation include, but are not limited to: encephalitis and other clinically important irAEs (e.g., vasculitis and sclerosing cholangitis).

Composition A may cause severe or life-threatening infusion reactions including severe hypersensitivity or anaphylaxis. Signs and symptoms usually develop during or shortly after drug infusion and generally resolve completely within 24 hours of completion of infusion. Dose modification and toxicity management guidelines on Composition A associated infusion reaction are provided in Table 11.

TABLE 11
Composition A Infusion Reaction Dose Modification and Treatment Guidelines

| NCI CTCAE Grade | Treatment | Premedication at Subsequent Dosing |
|---|---|---|
| Grade 1 | Increase monitoring of vital signs as | None |
| Mild reaction; infusion | medically indicated until the participant | |
| interruption not indicated; | is deemed medically stable in the opinion | |
| intervention not indicated | of the investigator | |
| Grade 2 | Stop Infusion | Participant may be premedicated 1.5 h |
| Requires therapy or | Additional appropriate medical therapy | (±30 minutes) prior to infusion of |
| infusion interruption but | may include but is not limited to: | study intervention with: |
| responds promptly to | IV fluids | Diphenhydramine 50 mg PO (or equivalent dose |
| symptomatic treatment | Antihistamines | of antihistamine). |
| (e.g., antihistamines, | NSAIDs | Acetaminophen 500-1000 mg PO (or equivalent |
| NSAIDs, narcotics, IV | Acetaminophen | dose of analgesic). |
| fluids); prophylactic | Narcotics | |
| medications indicated | Increase monitoring of vital signs as | |
| for ≤24 hrs | medically indicated until the participant | |
| | is deemed medically stable in the opinion | |
| | of the investigator. | |
| | If symptoms resolve within 1 hour of | |
| | stopping drug infusion, the infusion may | |
| | be restarted at 50% of the original | |
| | infusion rate (e.g., from 100 mL/hr to 50 | |
| | mL/hr). Otherwise dosing will be held | |
| | until symptoms resolve and the | |
| | participant should be premedicated for | |
| | the next scheduled dose. | |
| | Participants who develop Grade 2 | |
| | toxicity despite adequate premedication | |
| | should be permanently discontinued from | |
| | further study drug intervention. | |
| Grades 3 or 4 | Stop Infusion. | No subsequent dosing |
| Grade 3: | Additional appropriate medical therapy | |
| Prolonged (ie, not rapidly | may include but is not limited to: | |
| responsive to symptomatic | Epinephrine** | |
| medication and/or brief | IV fluids | |
| interruption of infusion); | Antihistamines | |
| recurrence of symptoms | NSAIDs | |
| following initial | Acetaminophen | |
| improvement; | Narcotics | |
| hospitalization indicated | Oxygen | |
| for other clinical sequelae | Pressors | |
| (e.g., renal impairment, | Corticosteroids | |
| pulmonary infiltrates) | Increase monitoring of vital signs as | |
| Grade 4: | medically indicated until the participant | |
| Life-threatening; pressor or | is deemed medically stable in the opinion | |
| ventilatory support | of the investigator. | |
| indicated | Hospitalization may be indicated. | |
| | **In cases of anaphylaxis, epinephrine | |
| | should be used immediately. Participant | |
| | is permanently discontinued from | |
| | further study drug intervention. | |

Appropriate resuscitation equipment should be available at the bedside and a physician readily available during the period of drug administration.
For further information, please refer to the Common Terminology Criteria for Adverse Events v5.0 (CTCAE) at http://ctep.cancer.gov

### Composition A Co-Formulation Administration

Composition A co-formulation will be administered using a 30-minute IV infusion every 3 weeks. Sites should make every effort to target infusion timing to be as close to 30 minutes as possible. However, given the variability of infusion pumps from site to site, a window of −5 minutes and +10 minutes is permitted (i.e., infusion time is 25 to 40 minutes).

### Efficacy Endpoint

This study will use objective response rate as the primary efficacy endpoint. Objective response rate is defined as the proportion of participants who have best response as complete response (CR) or partial response (PR). Responses are based on blinded independent central review (BICR) using RECIST 1.1.

This study will use duration of response (DOR), progression-free survival (PFS), overall survival (OS) and/or clinical benefit rate (CBR) as a secondary efficacy endpoint. For participants who demonstrate confirmed CR or PR per RECIST 1.1 as assessed by BICR, DOR is defined as the time from the first documented evidence of CR or PR until disease progression or death due to any cause, whichever occurs first.

PFS is defined as the time from the date of randomization to the first documented progressive disease (PD) per RECIST 1.1 by BICR, or death due to any cause, whichever occurs first.

OS is defined as the time from the date of randomization to the date of death from any cause.

CBR is defined as the percentage of participants who have achieved stable disease (SD) of ≥6 months or CR or PR based on assessments by BICR per RECIST 1.1.

Additionally, this study will use tumor size change as a tertiary/exploratory efficacy endpoint. Tumor size change is defined as the percent change in the sum of the diameters (longest for nonnodal lesions, short axis for nodal lesions) of the target lesion.

### Inclusion Criteria

A participant will be eligible for inclusion in the study if the participant:
1. Has a histologically confirmed diagnosis of locally advanced/metastatic ccRCC (with or without sarcomatoid features), i.e., Stage IV RCC per American Joint Committee on Cancer (AJCC).
2. Has received no prior systemic therapy for advanced RCC. Prior neoadjuvant/adjuvant therapy for RCC is acceptable if completed ≥12 months before randomization/allocation.
3. Has measurable disease per RECIST 1.1 as assessed by BICR. Lesions situated in a previously irradiated area are considered measurable if progression has been shown in such lesions.
4. Has a Karnofsky Performance Status (KPS) ≥70% assessed ≤10 days before randomization/allocation.
5. Is able to swallow oral medication.
6. Submits an archival tumor tissue sample or newly obtained core or excisional biopsy of a tumor lesion not previously irradiated. Formalin-fixed-paraffin-embedded (FFPE) tissue blocks are preferred to slides. Newly obtained biopsies are preferred to archived tissue.
7. Has adequate organ function as defined in Table 12. Specimens must be collected within 10 days before the start of study intervention.

TABLE 12
Adequate Organ Function Laboratory Values

| | |
|---|---|
| System | Laboratory Value |

Hematological

| | |
|---|---|
| Absolute neutrophil count (ANC) | ≥1500/μL |
| Platelets | ≥100 000/μL |
| Hemoglobin | ≥10 g/dL^{a} |

Renal

| | |
|---|---|
| Measured or calculated^{b} creatinine | ≥30 mL/min for participant with creatinine levels |
| clearance OR GFR | >1.5 × institutional ULN OR GFR criterion, e.g., 60 |
| | mL/min/1.73 m² |

Hepatic

| | |
|---|---|
| Total bilirubin | ≤1.5 × ULN OR direct bilirubin ≤ULN for participants |
| | with total bilirubin levels >1.5 × ULN |
| AST (SGOT) and ALT (SGPT) | ≤2.5 × ULN (≤5 × ULN for participants with |
| | liver metastases) |

Coagulation

| | |
|---|---|
| International normalized ratio (INR) OR | ≤1.5 × ULN unless participant is receiving anticoagulant |
| prothrombin time (PT) | therapy as long as PT or PTT is within therapeutic range of |
| Activated partial thromboplastin time (aPTT) | intended use of anticoagulants |

ALT (SGPT) = alanine aminotransferase (serum glutamic pyruvic transaminase); AST (SGOT) = aspartate aminotransferase (serum glutamic oxaloacetic transaminase); GFR = glomerular filtration rate; ULN = upper limit of normal.
^{a}Criteria must be met without erythropoietin dependency and without packed red blood cell (pRBC) transfusion within last 28 days.
^{b}Creatinine clearance (CrCl) should be calculated per institutional standard OR GRF criterion. Note that creatinine levels must be ≤1.5 × ULN.
Note:
This table includes eligibility-defining laboratory value requirements for treatment; laboratory value requirements should be adapted according to local regulations and guidelines for the administration of specific chemotherapies.

8. Participants receiving bone resorptive therapy (including, but not limited to bisphosphonate or receptor activator of nuclear factor-kB (RANK-L) inhibitor) must have therapy initiated at least 2 weeks before randomization/allocation.
9. Has adequately controlled blood pressure (BP) with or without antihypertensive medications, defined as BP ≤150/90 mm Hg with no change in antihypertensive medications within 1 week before randomization/allocation.
10. Is male or female, from 18 years to 120 years of age inclusive, at the time of signing the informed consent.
11. Male participants are eligible to participate if they agree to the following during the intervention period and for at least the time needed to eliminate each study intervention after the last dose of study intervention. The length of time required to continue contraception for each study intervention is as follows:
   No male contraception measures are required
   Be abstinent from heterosexual intercourse as their preferred and usual lifestyle (abstinent on a long-term and persistent basis) and agree to remain abstinent
OR
   Must agree to use contraception unless confirmed to be azoospermic (vasectomized or secondary to medical cause, documented from the site personnel's review of the participant's medical records, medical examination, or medical history interview) as detailed below:
      Agree to use a male condom plus partner use of an additional contraceptive method when having penile-vaginal intercourse with a woman/women of childbearing potential (WOCBP) who is not currently pregnant. Note: Men with a pregnant or breastfeeding partner must agree to remain abstinent from penile-vaginal intercourse or use a male condom during each episode of penile-vaginal penetration.
      Contraceptive use by men should be consistent with local regulations regarding the methods of contraception for those participating in clinical studies. If the contraception requirements in the local label for any of the study interventions is more stringent than the requirements above, the local label requirements are to be followed.
12. A female participant is eligible to participate if she is not pregnant or breastfeeding, and at least one of the following conditions applies:
   Is not a WOCBP
OR
   Is a WOCBP and:
      Uses a contraceptive method that is highly effective (with a failure rate of <1% per year), with low user dependency, or be abstinent from heterosexual intercourse as their preferred and usual lifestyle (abstinent on a long-term and persistent basis), during the intervention period and for at least the time needed to eliminate each study intervention after the last dose of study intervention. The length of time required to continue contraception is 120 days. The investigator should evaluate the potential for contraceptive method failure (i.e., noncompliance, recently initiated) in relationship to the first dose of study intervention. Contraceptive use by women should be consistent with local regulations regarding the methods of contraception for those participating in clinical studies. If the contraception requirements in the local label for any of the study interventions is more stringent than the requirements above, the local label requirements are to be followed.
      Has a negative highly sensitive pregnancy test (urine or serum as required by local regulations) within 24 hours before the first dose of study intervention. If a urine test cannot be confirmed as negative (e.g., an ambiguous result), a serum pregnancy test is required. In such cases, the participant must be excluded from participation if the serum pregnancy result is positive.
      Abstains from breastfeeding during the study intervention period and for at least 120 days after study intervention.
      Has had her medical history, menstrual history, and recent sexual activity reviewed by the investigator to decrease the risk for inclusion of a woman with an early undetected pregnancy.

### Exclusion Criteria

The participant must be excluded from the study if the participant:
1. Has clinically significant cardiovascular disease within 12 months from first dose of study intervention, including New York Heart Association Class III or IV congestive heart failure, unstable angina, myocardial infarction, cerebral vascular accident, or cardiac arrhythmia associated with hemodynamic instability. Medically controlled arrhythmia would be permitted.
2. Prolongation of QT interval corrected by Fridericia's formula (QTcF) interval to >480 ms.
3. Has a left ventricular ejection fraction (LVEF) below the institutional (or local laboratory) normal range as determined by multigated acquisition scan (MUGA) or echocardiogram (ECHO).
4. Has had major surgery within 3 weeks before first dose of study interventions. Note: Adequate wound healing after major surgery must be assessed clinically, independent of time elapsed for eligibility.
5. Has urine protein ≥1 g/24 hours.
   Note: Participants with proteinuria >1+ on urine dipstick testing/urinalysis will undergo 24-h urine collection for quantitative assessment of proteinuria.
6. Has a history of interstitial lung disease, history of (noninfectious) pneumonitis that required steroids, or has current pneumonitis.
7. Has symptomatic pleural effusion (for example cough, dyspnea, pleuritic chest pain). A participant who is clinically stable after treatment for these conditions (including therapeutic thoraco- or paracentesis) is eligible.
8. Has a history of inflammatory bowel disease.
9. Has preexisting ≥Grade 3 GI or non-GI fistula.
10. Has malabsorption due to prior gastrointestinal (GI) surgery or GI disease.
11. Active hemoptysis (bright red blood of at least 0.5 teaspoon) within 3 weeks prior to the first dose of study intervention.
12. Has received prior radiotherapy within 2 weeks of start of study intervention or radiation-related toxicities requiring corticosteroids. Two weeks or fewer of palliative radiotherapy for non-central nervous system (CNS) disease, with a 1-week washout, is permitted.
13. Has received a live or live attenuated vaccine within 30 days before the first dose of study intervention. Killed vaccines are allowed.
14. Has received an investigational agent or has used an investigational device within 4 weeks prior to study intervention administration.
15. Participants who have been previously allocated/randomized to study intervention in any sub study of protocol MK-3475-U03.
16. Has a diagnosis of immunodeficiency or is receiving chronic systemic steroid therapy (in dosing exceeding 10 mg daily of prednisone equivalent) or any other form of immunosuppressive therapy within 7 days prior the first dose of study intervention.
17. Has a known additional malignancy that is progressing or has required active treatment within the past 3 years. Participants with basal cell carcinoma of the skin, squamous cell carcinoma of the skin, or carcinoma in situ (e.g., breast carcinoma, cervical cancer in situ) that have undergone potentially curative therapy are not excluded.
18. Has known active CNS metastases and/or carcinomatous meningitis. Participants with previously treated brain metastases may participate provided they are radiologically stable, (i.e., without evidence of progression) for at least 4 weeks by repeat imaging (note that the repeat imaging should be performed during study screening), clinically stable and without requirement of steroid treatment for at least 14 days prior to first dose of study intervention.
19. Has radiographic evidence of encasement or invasion of a major blood vessel, or of intratumoral cavitation. The degree of tumor invasion/infiltration of major blood vessels should be considered because of the potential risk of severe hemorrhage associated with tumor shrinkage/necrosis after lenvatinib therapy.
20. Has a history of hypersensitivity reaction to any of Composition A and belzutifan. For example, but not limited to:
   Has a severe hypersensitivity (≥Grade 3) to pembrolizumab and/or any of its excipients.
21. Has an active autoimmune disease that has required systemic treatment in the past 2 years except replacement therapy (e.g., thyroxine, insulin, or physiologic corticosteroid). Participants with vitiligo, Sjøgren's syndrome, Type I diabetes, or resolved childhood asthma/atopy will not be excluded from the study. Participants requiring intermittent use of bronchodilators, inhaled steroids, or local steroid injections would not be excluded from the study. Participants with hypothyroidism or adrenal or pituitary insufficiency who are stable on hormone replacement will not be excluded from the study.
22. Has an active infection requiring systemic therapy.
23. Has a known history of human immunodeficiency virus (HIV) infection. No HIV testing is required unless mandated by local health authority.
24. Has a known history of Hepatitis B (defined as HBsAg reactive) or known active Hepatitis C virus (defined as HCV RNA is detected) infection. No testing for Hepatitis B and Hepatitis C is required unless mandated by local health authority.
25. Has any of the following:
   A pulse oximeter reading <92% at rest, or
   Requires intermittent supplemental oxygen, or
   Requires chronic supplemental oxygen.
26. Has a history or current evidence of any condition, therapy, or laboratory abnormality that might confound the results of the study, interfere with the participant's participation for the full duration of the study, or is not in the best interest of the participant to participate, in the opinion of the treating investigator.
27. Has a known psychiatric or substance abuse disorder that would interfere with the participant's ability to cooperate with the requirements of the study.
28. Has had an allogenic tissue/solid organ transplant.

### Assessment and Decision at RECIST 1.1 Progression

RECIST 1.1 will be used as the primary measure for assessment of tumor response, date of disease progression, and as a basis for all protocol guidelines related to disease status (e.g., discontinuation of study intervention). Although RECIST 1.1 references a maximum of 5 target lesions in total and 2 per organ, this protocol allows a maximum of 10 target lesions in total and 5 per organ, if clinically relevant to enable a broader sampling of tumor burden.

If disease progression is established by the investigator, the process continues as follows:
investigator judgement will determine action
if the participant is clinically stable and study intervention is to continue, communication with the sponsor is required and a reconsent addendum must be signed. In addition, the following are to occur:
   continue scans per protocol schedule (the next scheduled scan should be ≥4 weeks from most recent scan acquired)
   send scans to iCRO
      For the purpose of this decision process, lack of clinical stability is defined as:
unacceptable toxicity
clinical signs or symptoms indicating clinically significant disease progression
decline in performance status
rapid disease progression or threat to vital organs or critical anatomical sites (e.g., CNS metastasis, respiratory failure due to tumor compression, spinal cord compression) requiring urgent alternative medical intervention.

### Assessment at the Confirmatory Imaging

In addition to survival, efficacy will be assessed based on imaging evaluation of changes in tumor burden over time, until the participant is discontinued from the study or goes into Survival Follow-up. The process for image collection and transmission to the iCRO can be found in the SIM. Tumor imaging is strongly preferred to be acquired by CT. For the abdomen and pelvis, contrast-enhanced MRI may be used when CT with iodinated contrast is contraindicated, or when mandated by local practice. The same imaging technique should be used in a participant throughout the study to optimize the reproducibility of the assessment of existing and new tumor burden and improve the accuracy of the response assessment based on imaging. Note: for the purposes of assessing tumor imaging, the term “investigator” refers to the local investigator at the site and/or the radiological reviewer at the site or at an offsite facility.

If brain imaging is performed, magnetic resonance imaging is preferred; however, CT imaging will be acceptable, if MRI is medically contraindicated.

Bone scans may be performed to evaluate bone metastases. Any supplemental imaging performed to support a positive or negative bone scan, such as plain X-rays acquired for correlation, should be submitted to the iCRO.

Other imaging modalities that may be collected, submitted to the iCRO, and included in the response assessment include FDG-PET if acquired. Other types of medical imaging (such as ultrasound) should not be submitted to the iCRO and will not be included in response assessment.

### Discontinuation

Participants who discontinue study intervention before completion of the treatment period should be encouraged to continue to be followed for all remaining study visits.

Participants who withdraw from the study should be encouraged to complete all applicable activities scheduled for the final study visit at the time of withdrawal. Any AEs that are present at the time of withdrawal should be followed in accordance with the safety requirements.

### Safety Endpoints

The safety endpoints include AEs, DLTs, SAEs, and study intervention discontinuation due to AEs. In addition, safety and tolerability will be assessed by clinical review of all relevant parameters including AEs, laboratory tests, vital signs, and ECGs.

### Adverse Event (AE)

An AE is any untoward medical occurrence in a clinical study participant, temporally associated with the use of study intervention, whether or not considered related to the study intervention. An AE can therefore be any unfavorable and unintended sign (including an abnormal laboratory finding), symptom, or disease (new or exacerbated) temporally associated with the use of a study intervention. For purposes of AE definition, study intervention (also referred to as Sponsor's product) includes any pharmaceutical product, biological product, vaccine, diagnostic agent, medical device, combination product, or protocol specified procedure whether investigational or marketed (including placebo, active comparator product, or run-in intervention), manufactured by, licensed by, provided by, or distributed by the Sponsor for human use in this study.

The following are included as AEs:
Any abnormal laboratory test results (hematology, clinical chemistry, or urinalysis) or other safety assessments (e.g., ECG, radiological scans, vital signs measurements), including those that worsen from baseline, considered clinically significant in the medical and scientific judgment of the investigator.
Exacerbation of a chronic or intermittent preexisting condition including either an increase in frequency and/or intensity of the condition.
New conditions detected or diagnosed after study intervention administration even though it may have been present before the start of the study.
Signs, symptoms, or the clinical sequelae of a suspected drug-drug interaction.
Signs, symptoms, or the clinical sequelae of a suspected overdose of either study intervention or a concomitant medication.
For all reports of overdose (whether accidental or intentional) with an associated AE, the AE term should reflect the clinical symptoms or abnormal test result. An overdose without any associated clinical symptoms or abnormal laboratory results is reported using the terminology “accidental or intentional overdose without adverse effect.”
Any new cancer (that is not a condition of the study). Progression of the cancer under study is not a reportable event.
   The following events do not meet the AE definition for the purpose of this study:
Medical or surgical procedure (e.g., endoscopy, appendectomy): the condition that leads to the procedure is the AE.
Situations in which an untoward medical occurrence did not occur (social and/or convenience admission to a hospital).
Anticipated day-to-day fluctuations of preexisting disease(s) or condition(s) present or detected at the start of the study that do not worsen.
Surgical procedure(s) planned prior to informed consent to treat a preexisting condition that has not worsened.

### Serious Adverse Event (SAE)

If an event is not an AE per the above, then it cannot be an SAE even if serious conditions are met. An SAE is defined as any untoward medical occurrence that, at any dose:
Results in death.
Is life-threatening. The term “life-threatening” in the definition of “serious” refers to an event in which the participant was at risk of death at the time of the event. It does not refer to an event, which hypothetically might have caused death, if it were more severe.
Requires inpatient hospitalization or prolongation of existing hospitalization. Hospitalization is defined as an inpatient admission, regardless of length of stay, even if the hospitalization is a precautionary measure for continued observation. (Note: Hospitalization for an elective procedure to treat a pre-existing condition that has not worsened is not an SAE. A pre-existing condition is a clinical condition that is diagnosed prior to the use of an product and is documented in the participant's medical history.
Results in persistent or significant disability/incapacity. The term disability means a substantial disruption of a person's ability to conduct normal life functions. This definition is not intended to include experiences of relatively minor medical significance such as uncomplicated headache, nausea, vomiting, diarrhea, influenza, and accidental trauma (e.g., sprained ankle) that may interfere with or prevent everyday life functions but do not constitute a substantial disruption.
Is a congenital anomaly/birth defect. In offspring of participant taking the product regardless of time to diagnosis.
   Other important medical events. Medical or scientific judgment should be exercised in deciding whether SAE reporting is appropriate in other situations such as important medical events that may not be immediately life-threatening or result in death or hospitalization but may jeopardize the participant or may require medical or surgical intervention to prevent 1 of the other outcomes listed in the above definition. These events should usually be considered serious. Examples of such events include invasive or malignant cancers, intensive treatment in an emergency room or at home for allergic bronchospasm, blood dyscrasias or convulsions that do not result in hospitalization, or development of drug dependency or drug abuse.

Table 13 outlines procedures and assessments to be performed. Specific procedures and assessments can be found in Table 14.

TABLE 13
Schedule of Activities

| | | | | | | | | | | | Posttreatment | | | |
|---|---|---|---|---|---|---|---|---|---|---|---|---|---|---|
| Trial Period | | Treatment Cycles | | | | | | | | | Follow-up | | | |
| Treatment Cycle/ | | (Cycle = 42 days) | | | | | | | | | Safety | | Survival | |
| Visit Title | | | | | | | | | | End of | Follow-up | Efficacy | Follow-up | |
| Cycle Day | Screening | 1 | | | | 2 | | 3+ | | Treatment | 30 days from | Follow-up | Every 12 | |
| Scheduling | Screening | 1 | 8 | 15 | 22 | 1 | 22 | 1 | 22 | Discon | last dose | Per Imaging | weeks | |
| Window (Days) | −28 to −1 | +3 | ±3 | ±3 | ±3 | ±3 | ±3 | ±3 | ±3 | EOT | (±7 days) | Schedule | (±7 days) | Notes |

Administrative Procedures

| Informed | X | | | | | | | | | | | Informed consent |
|---|---|---|---|---|---|---|---|---|---|---|---|---|
| Consent | | | | | | | | | | | | must be obtained |
| | | | | | | | | | | | | and documented |
| | | | | | | | | | | | | before any |
| | | | | | | | | | | | | protocol-specific |
| | | | | | | | | | | | | procedures. |
| | | | | | | | | | | | | If the investigator |
| | | | | | | | | | | | | plans to treat |
| | | | | | | | | | | | | beyond disease |
| | | | | | | | | | | | | progression, |
| | | | | | | | | | | | | additional |
| | | | | | | | | | | | | consent is required. |
| Inclusion | X | | | | | | | | | | | Verification |
| Exclusion | | | | | | | | | | | | documented by |
| Criteria | | | | | | | | | | | | investigator. |
| Participant | X | X | | | | | | | | | | At Visit 1, site |
| Identification | | | | | | | | | | | | personnel should |
| Card | | | | | | | | | | | | add the |
| | | | | | | | | | | | | randomization |
| | | | | | | | | | | | | or allocation |
| | | | | | | | | | | | | number to the |
| | | | | | | | | | | | | participant ID |
| | | | | | | | | | | | | card. |
| Demographics | X | | | | | | | | | | | Medical History |
| and Medical | | | | | | | | | | | | includes tobacco |
| History | | | | | | | | | | | | use and family |
| | | | | | | | | | | | | of history premature |
| | | | | | | | | | | | | cardiovascular |
| | | | | | | | | | | | | disease. |
| RCC Disease | X | | | | | | | | | | | |
| History | | | | | | | | | | | | |
| Prior/ | X | X | X | X | X | X | X | X | X | | | |
| Concomitant | | | | | | | | | | | | |
| Medication | | | | | | | | | | | | |
| Review | | | | | | | | | | | | |
| Intervention | | X | | | | | | | | | | |
| Randomization/ | | | | | | | | | | | | |
| Allocation | | | | | | | | | | | | |
| Posttreatment | | | | | | | | X | X | X | X | |
| Anticancer | | | | | | | | | | | | |
| Therapy Status | | | | | | | | | | | | |
| Vital Status | | ←------------------------------→ | | | | | | | | | X | After investigator |
| | | | | | | | | | | | | determined PD or |
| | | | | | | | | | | | | start of new |
| | | | | | | | | | | | | anticancer |
| | | | | | | | | | | | | treatment. |
| | | | | | | | | | | | | In addition, |
| | | | | | | | | | | | | on Sponsor request, |
| | | | | | | | | | | | | participants may be |
| | | | | | | | | | | | | contacted for vital |
| | | | | | | | | | | | | status at any time |
| | | | | | | | | | | | | during the course |
| | | | | | | | | | | | | of the study. |
| Tumor Imaging | | | | | | | | | | | | Imaging should |
| | | | | | | | | | | | | follow calendar |
| | | | | | | | | | | | | days from |
| | | | | | | | | | | | | randomization/ |
| | | | | | | | | | | | | allocation |
| | | | | | | | | | | | | and should not be |
| | | | | | | | | | | | | adjusted for dose |
| | | | | | | | | | | | | delays or cycle |
| | | | | | | | | | | | | starts. |
| | | | | | | | | | | | | Unscheduled |
| | | | | | | | | | | | | imaging can |
| | | | | | | | | | | | | be performed as |
| | | | | | | | | | | | | clinically indicated. |
| CT/MRI of | X | X | | | | | | | | X | | Use the same |
| chest, abdomen, | | | | | | | | | | | | imaging modality |
| and pelvis | | | | | | | | | | | | for all evaluations. |
| | | | | | | | | | | | | Perform at Week |
| | | | | | | | | | | | | 12 (84 days ± |
| | | | | | | | | | | | | 7 days). Repeat |
| | | | | | | | | | | | | Q6W (42 days ± |
| | | | | | | | | | | | | 7 days) for the |
| | | | | | | | | | | | | first 54 weeks |
| | | | | | | | | | | | | and Q12W (84 |
| | | | | | | | | | | | | days ± 7 |
| | | | | | | | | | | | | days) thereafter. |
| Bone Scan | X | X | | | | | | | | X | | Perform screening |
| | | | | | | | | | | | | bone scan within |
| | | | | | | | | | | | | 28 days before |
| | | | | | | | | | | | | randomization/ |
| | | | | | | | | | | | | allocation. |
| | | | | | | | | | | | | Screening bone |
| | | | | | | | | | | | | scans do not need |
| | | | | | | | | | | | | to be repeated |
| | | | | | | | | | | | | if performed |
| | | | | | | | | | | | | for standard of |
| | | | | | | | | | | | | care within 42 |
| | | | | | | | | | | | | days before |
| | | | | | | | | | | | | randomization/ |
| | | | | | | | | | | | | allocation. |
| | | | | | | | | | | | | If positive at |
| | | | | | | | | | | | | baseline, continue |
| | | | | | | | | | | | | with bone scans |
| | | | | | | | | | | | | on study starting |
| | | | | | | | | | | | | at Week 18 (±7 |
| | | | | | | | | | | | | days), then Q12W |
| | | | | | | | | | | | | (±7 days) |
| | | | | | | | | | | | | through Week 78 |
| | | | | | | | | | | | | (Weeks 30, 42, 54, |
| | | | | | | | | | | | | 66, 78), then Q24W |
| | | | | | | | | | | | | (±7 days) thereafter. |
| | | | | | | | | | | | | If imaging was |
| | | | | | | | | | | | | obtained within 28 |
| | | | | | | | | | | | | days before discon, |
| | | | | | | | | | | | | scan at discon |
| | | | | | | | | | | | | is not mandatory. |
| | | | | | | | | | | | | Perform for |
| | | | | | | | | | | | | participants at time |
| | | | | | | | | | | | | of CR if positive |
| | | | | | | | | | | | | at baseline. |
| | | | | | | | | | | | | Schedule should be |
| | | | | | | | | | | | | followed regardless |
| | | | | | | | | | | | | of treatment delays. |
| Brain Scan | X | | | | | | | | | | | Only perform brain |
| | | | | | | | | | | | | scan at screening |
| | | | | | | | | | | | | for participants |
| | | | | | | | | | | | | with previously |
| | | | | | | | | | | | | documented brain |
| | | | | | | | | | | | | metastases to |
| | | | | | | | | | | | | ensure they |
| | | | | | | | | | | | | are stable. |
| | | | | | | | | | | | | On-study brain |
| | | | | | | | | | | | | imaging should be |
| | | | | | | | | | | | | performed if |
| | | | | | | | | | | | | there are clinical |
| | | | | | | | | | | | | symptoms leading to |
| | | | | | | | | | | | | suspicion of disease |
| | | | | | | | | | | | | progression |
| | | | | | | | | | | | | associated |
| | | | | | | | | | | | | with brain lesions |
| | | | | | | | | | | | | (new brain lesions |
| | | | | | | | | | | | | or enlargement of |
| | | | | | | | | | | | | existing brain |
| | | | | | | | | | | | | lesions) or to |
| | | | | | | | | | | | | confirm a CR (if |
| | | | | | | | | | | | | other lesions |
| | | | | | | | | | | | | indicate CR and |
| | | | | | | | | | | | | brain lesion existed |
| | | | | | | | | | | | | at baseline). |

Safety Procedures

| | | | | | | | | | | | | |
|---|---|---|---|---|---|---|---|---|---|---|---|---|
| AE/SAE | X | X | | X | X | X | X | X | X | X | X | Record all AEs |
| Review | | | | | | | | | | | | occurring within 30 |
| | | | | | | | | | | | | days after last dose |
| | | | | | | | | | | | | of study treatment. |
| | | | | | | | | | | | | After 30 days, |
| | | | | | | | | | | | | record all |
| | | | | | | | | | | | | SAEs occurring |
| | | | | | | | | | | | | up to 90 days after |
| | | | | | | | | | | | | last dose of study |
| | | | | | | | | | | | | treatment or 30 |
| | | | | | | | | | | | | days following |
| | | | | | | | | | | | | last dose if the |
| | | | | | | | | | | | | participant initiates |
| | | | | | | | | | | | | new anticancer |
| | | | | | | | | | | | | therapy, whichever |
| | | | | | | | | | | | | comes first. |
| | | | | | | | | | | | | Any treatment- |
| | | | | | | | | | | | | related SAEs |
| | | | | | | | | | | | | must be reported |
| | | | | | | | | | | | | regardless of time |
| | | | | | | | | | | | | they occur. |
| Full Physical | X | | | | | | | | | X | | |
| Examination | | | | | | | | | | | | |
| Directed | | X | | | X | X | X | X | X | | X | |
| Physical | | | | | | | | | | | | |
| Examination | | | | | | | | | | | | |
| Height | X | | | | | | | | | | | |
| Weight | X | X | | | X | X | X | X | X | | | |
| Vital | X | X | | X | X | X | X | X | X | X | X | Heart rate, RR, |
| Signs | | | | | | | | | | | | temperature, pulse |
| | | | | | | | | | | | | oximetry |
| BP | X | | | | | | | | | | | DBP, SBP |
| 12-lead | X | | | | | | | | | | | |
| ECG (local) | | | | | | | | | | | | |
| Echocardiogram | X | | | | | | | | | | | MUGA scan (using |
| or MUGA | | | | | | | | | | | | technetium-based |
| Scan (local) | | | | | | | | | | | | tracer) orECHO |
| | | | | | | | | | | | | will be performed |
| | | | | | | | | | | | | to assess LVEF. |
| | | | | | | | | | | | | MUGA is preferred; |
| | | | | | | | | | | | | however, whichever |
| | | | | | | | | | | | | modality is used |
| | | | | | | | | | | | | for an individual |
| | | | | | | | | | | | | participant at |
| | | | | | | | | | | | | baseline should |
| | | | | | | | | | | | | be repeated for |
| | | | | | | | | | | | | all subsequent |
| | | | | | | | | | | | | LVEF assessments. |
| | | | | | | | | | | | | After randomization/ |
| | | | | | | | | | | | | allocation: |
| | | | | | | | | | | | | For participants |
| | | | | | | | | | | | | who are assigned |
| | | | | | | | | | | | | to an arm that |
| | | | | | | | | | | | | does not contain |
| | | | | | | | | | | | | lenvatinib, |
| | | | | | | | | | | | | ECHO may be |
| | | | | | | | | | | | | performed as |
| | | | | | | | | | | | | clinically indicated. |
| ECOG | X | X | | X | X | X | X | X | X | X | X | |
| Performance | | | | | | | | | | | | |
| Status | | | | | | | | | | | | |
| KPS | X | | | | | | | | | | | At screening, |
| | | | | | | | | | | | | perform ≤10 |
| | | | | | | | | | | | | days before |
| | | | | | | | | | | | | randomization/ |
| | | | | | | | | | | | | allocation |
| Urine dipstick | X | | | | | | | | | | | After |
| testing for | | | | | | | | | | | | randomization/ |
| protein | | | | | | | | | | | | allocation: |
| | | | | | | | | | | | | For participants who |
| | | | | | | | | | | | | are assigned to an |
| | | | | | | | | | | | | arm that does not |
| | | | | | | | | | | | | contain lenvatinib, |
| | | | | | | | | | | | | urine dipstick |
| | | | | | | | | | | | | testing for |
| | | | | | | | | | | | | protein may be |
| | | | | | | | | | | | | performed as |
| | | | | | | | | | | | | clinically |
| | | | | | | | | | | | | indicated. |
| Urine or Serum | X | X | | | X | X | X | X | X | X | X | Serum test is only |
| hCG (WOCBP | | | | | | | | | | | | required if urine |
| only, per | | | | | | | | | | | | test is positive |
| local SOP) | | | | | | | | | | | | or not evaluable. |
| | | | | | | | | | | | | WOCBP require |
| | | | | | | | | | | | | negative test before |
| | | | | | | | | | | | | randomization/ |
| | | | | | | | | | | | | allocation. |
| | | | | | | | | | | | | If more than 24 |
| | | | | | | | | | | | | hours have elapsed |
| | | | | | | | | | | | | before first dose of |
| | | | | | | | | | | | | study intervention, |
| | | | | | | | | | | | | another pregnancy |
| | | | | | | | | | | | | test is required |
| | | | | | | | | | | | | before starting |
| | | | | | | | | | | | | study intervention. |
| | | | | | | | | | | | | Thereafter, |
| | | | | | | | | | | | | pregnancy |
| | | | | | | | | | | | | tests will be |
| | | | | | | | | | | | | conducted Q3W. |
| HBV, HCV, | X | | | | | | | | | | | ONLY perform if |
| and HIV | | | | | | | | | | | | required by local |
| testing | | | | | | | | | | | | health authority. |
| Hematology | X | | X | | X | X | X | X | X | X | X | Screening samples |
| Erythropoietin | X | | | | | | | | | | | must be collected |
| Comprehensive | X | | X | | X | X | X | X | X | X | X | ≤10 days before |
| Chemistry Panel | | | | | | | | | | | | start of study |
| Urinalysis | X | | X | | X | X | X | X | X | X | X | intervention. |
| T3 or FT3, FT4 | X | | | | X | X | X | X | X | X | X | |
| and TSH | | | | | | | | | | | | |
| Coagulation tests | X | | | | | | | | | | | Screening samples |
| (PT/INR and | | | | | | | | | | | | must be collected |
| aPTT or PTT) | | | | | | | | | | | | ≤10 days before |
| | | | | | | | | | | | | start of study |
| | | | | | | | | | | | | intervention. |
| | | | | | | | | | | | | After |
| | | | | | | | | | | | | randomization/ |
| | | | | | | | | | | | | allocation: |
| | | | | | | | | | | | | For participants |
| | | | | | | | | | | | | who are assigned |
| | | | | | | | | | | | | to an arm that |
| | | | | | | | | | | | | does not contain |
| | | | | | | | | | | | | belzutifan, |
| | | | | | | | | | | | | coagulation |
| | | | | | | | | | | | | tests should be |
| | | | | | | | | | | | | performed as |
| | | | | | | | | | | | | clinically |
| | | | | | | | | | | | | indicated for |
| | | | | | | | | | | | | participants taking |
| | | | | | | | | | | | | anticoagulants. |

Biomarker Assessments

| | | | |
|---|---|---|---|
| Archival or | X | | |
| Newly Obtained | | | |
| Tissue Collection | | | |
| for Biomarkers | | | |
| Blood for | | X | Collect predose. |
| Genetic | | | |
| Analyses | | | |

Abbreviations: AE = adverse event; aPTT = activated partial thromboplastin time; BP = blood pressure; CR = complete response; CT = computerized tomography; DBP = diastolic blood pressure; Discon = discontinuation; ECG = electrocardiogram; ECHO = echocardiogram; ECOG = Eastern Cooperative Oncology Group; EOT = end of treatment; HBV = Hepatitis B virus; hCG = human chorionic gonadotropin; HCV = Hepatitis C virus; HIV = human immunodeficiency virus; HR = heart rate; ID = identification; INR = international normalized ratio; KPS = Karnofsy Performance Status; LVEF = left ventricular ejection fraction; MRI = magnetic resonance imaging; MUGA = multigated acquisition; PD = progressive disease; PT = prothrombin time; PTT = partial thromboplastin time; Q = every; RCC = renal cell carcinoma; RR = respiratory rate; SAE = serious adverse event; SBP = systolic blood pressure; SOP = standard operating procedure; T3 = triiodothyronine; FT3 = free triiodothyronine: FT4 = free thyroxine; TSH = thyroid-stimulating hormone; W = weeks; WOCBP = women of childbearing potential.

TABLE 14
Schedule of Activities

| | | | | | | | | | | Posttreatment | | |
|---|---|---|---|---|---|---|---|---|---|---|---|---|
| | Treatment Cycles | | | | | | | | | Follow-up | | |
| | (Cycle = 42 days) | | | | | | | | | Safety | Efficacy | |
| Cycle | | | | | | | | | End of | Follow-up | Follow-up | |
| Cycle Day | 1 | | | | 2 | | 3+ | | Treatment | 30 days from | Per | |
| Scheduling | 1 | 8 | 15 | 22 | 1 | 22 | 1 | 22 | Discon | last dose | Imaging | |
| Window | +3 | ±3 | ±3 | ±3 | ±3 | ±3 | ±3 | ±3 | EOT | (±7 days) | Schedule | Notes |
| Composition A | X | | | X | X | X | X | X | | | z | Maximum of 35 doses (2 years) |
| Administration | | | | | | | | | | | | Composition A will be |
| | | | | | | | | | | | | administered as an IV infusion |
| | | | | | | | | | | | | over 30 minutes. |
| Belzutifan | X | | | X | X | X | X | X | | | | The C1D1 dose of belzutifan |
| Dispensed | | | | | | | | | | | | will be taken fasted (at least 2 |
| | | | | | | | | | | | | hours after a meal). |
| | | | | | | | | | | | | Belzutifan will be administered |
| | | | | | | | | | | | | at least 30 minutes after |
| | | | | | | | | | | | | completion of Composition A |
| | | | | | | | | | | | | infusion. On clinic visit days, |
| | | | | | | | | | | | | participants should not take |
| | | | | | | | | | | | | belzutifan before their |
| | | | | | | | | | | | | appointment; participants should |
| | | | | | | | | | | | | be instructed to bring their |
| | | | | | | | | | | | | belzutifan to the clinic. |
| | | | | | | | | | | | | Belzutifan will be dosed until |
| | | | | | | | | | | | | PD is documented or a |
| | | | | | | | | | | | | discontinuation criterion is met. |
| Belzutifan | | | | X | X | X | X | X | X | | | |
| Container | | | | | | | | | | | | |
| Returned | | | | | | | | | | | | |
| Composition A | X | | | X | X | X | X | | X | X | | Collect predose samples at C1D1 |
| ADA/NAb | | | | | | | | | | | | and C1D22, C2D1, C2D22, |
| | | | | | | | | | | | | C3D1, C4D1 at EOT, and at the |
| | | | | | | | | | | | | Safety Follow-up Visit. |
| Belzutifan PK | X | X | | X | X | | | | | | | Dose on C1D1 should be taken |
| | | | | | | | | | | | | fasted (at least 2 hours after a |
| | | | | | | | | | | | | meal); food should be withheld |
| | | | | | | | | | | | | for 1-hour postdose. PK samples |
| | | | | | | | | | | | | should be collected on C1D1 at |
| | | | | | | | | | | | | predose, 1, 2, and 4 hours |
| | | | | | | | | | | | | postdose (within 30 minutes). |
| | | | | | | | | | | | | Predose samples should be |
| | | | | | | | | | | | | collected on C1D8, |
| | | | | | | | | | | | | C1D22, and C2D1. If treatment |
| | | | | | | | | | | | | is interrupted or discontinued |
| | | | | | | | | | | | | before C2D1, every effort should |
| | | | | | | | | | | | | be taken to collect the PK |
| | | | | | | | | | | | | sample for C2D1 time point at |
| | | | | | | | | | | | | the time of treatment interruption |
| | | | | | | | | | | | | or discontinuation. |
| Composition A | X | | | X | X | X | X | | X | X | | Collect predose samples at |
| PK | | | | | | | | | | | | C1D1, C1D22, C2D1, C2D22, |
| | | | | | | | | | | | | C3D1, C4D1, at EOT, and at the |
| | | | | | | | | | | | | Safety Follow-up Visit. |
| | | | | | | | | | | | | Additional postdose samples |
| | | | | | | | | | | | | should be taken within 30 |
| | | | | | | | | | | | | minutes after the end of |
| | | | | | | | | | | | | Composition A infusion at C1D1 |
| | | | | | | | | | | | | and C2D1. |
| Blood for RNA | X | | | X | | | X | | X | | | Collect blood at C1D1 predose, |
| Analyses | | | | | | | | | | | | C1D22 predose, C3D1 predose, |
| | | | | | | | | | | | | and EOT. |
| Blood for ctDNA | X | | | X | X | | X | | X | | | Collect predose. Collect at |
| Analyses | | | | | | | | | | | | C1D1, C1D22, C2D1, and |
| | | | | | | | | | | | | C3D1. After C3D1 collection, |
| | | | | | | | | | | | | the blood for ctDNA samples |
| | | | | | | | | | | | | should be collected Q6W |
| | | | | | | | | | | | | (C4D1, C5D1, C6D1, etc.) until |
| | | | | | | | | | | | | C10D1. After C10D1, blood for |
| | | | | | | | | | | | | ctDNA will be collected Q12W |
| | | | | | | | | | | | | (C12D1, C14D1, C16D1, etc.), |
| | | | | | | | | | | | | and at EOT. Collection of |
| | | | | | | | | | | | | ctDNA samples is not required |
| | | | | | | | | | | | | after EOT. |
| Blood for Plasma | X | | | X | | | | | X | | | Collect blood at C1D1 predose, |
| Biomarker | | | | | | | | | | | | C1D22 predose and EOT. |
| Analyses | | | | | | | | | | | | |
| Blood for Serum | X | X | | X | X | | | | X | | | Dose on C1D1 should be taken |
| Biomarker | | | | | | | | | | | | fasted (at least 2 hours after a |
| Analyses | | | | | | | | | | | | meal); food should be withheld |
| | | | | | | | | | | | | for 1-hour postdose. Blood for |
| | | | | | | | | | | | | Serum Biomarker Analyses |
| | | | | | | | | | | | | samples should be collected on |
| | | | | | | | | | | | | C1D1 at predose, 1, 2, and 4 |
| | | | | | | | | | | | | hours postdose. Predose samples |
| | | | | | | | | | | | | should be collected on C1D8, |
| | | | | | | | | | | | | C1D22, and C2D1, and at EOT. |
| | | | | | | | | | | | | If treatment is interrupted or |
| | | | | | | | | | | | | discontinued before C2D1, every |
| | | | | | | | | | | | | effort should be taken to collect |
| | | | | | | | | | | | | the Blood for Serum Biomarker |
| | | | | | | | | | | | | Analyses sample for C2D1 time |
| | | | | | | | | | | | | point at the time of treatment |
| | | | | | | | | | | | | interruption or discontinuation. |
| BP | X | | X | X | X | X | X | X | X | X | | DBP, SBP |
| 12-lead ECG | X | | | X | X | X | X | X | X | X | | Perform ECG on D1 and D22 for |
| (local) | | | | | | | | | | | | Cycles 1, 2, and 3. Then D1 of |
| | | | | | | | | | | | | every cycle, EOT, and Safety |
| | | | | | | | | | | | | Follow-up. |
| | | | | | | | | | | | | If belzutifan is discontinued, |
| | | | | | | | | | | | | ECGs are only required at the |
| | | | | | | | | | | | | EOT and Safety Follow-up |
| | | | | | | | | | | | | visits. |
| Coagulation tests | | | | X | X | X | X | X | X | X | | |
| (PT/INR and | | | | | | | | | | | | |
| aPTT or PTT) | | | | | | | | | | | | |

### Example 2

The present Example describes preclinical data using a TIGIT antagonist and a PD-1 antagonist in combination with belzutifan, using mouse syngeneic models.

Prior to treatment initiation, female BALB/cAnn mice aged 7 weeks weighing between 18 to 21 grams were anesthetized and injected subcutaneously into the rear flank with 0.3×10⁶ log-phase sub-confluent CT26 cells. Mice were pair-matched into treatment groups consisting of 10 mice per group when the mean tumor volume of inoculated CT26 tumors reached approximately 150 mm³.

Treatment groups consisted of:
1) Isotype mouse IgG1 (mIgG1) antibody+Isotype mouse IgG2a (mIgG2a) antibody+0.5% methylcellulose (vehicle);
2) anti-PD-1 mIgG1 antibody+Isotype mIgG2a antibody+vehicle;
3) anti-TIGIT mIgG2a antibody+Isotype mIgG1 antibody+vehicle;
4) Isotype mIgG1 antibody+Isotype mIgG2a antibody+belzutifan;
5) anti-PD-1 mIgG1 antibody+anti-TIGIT mIgG2a antibody+vehicle;
6) anti-PD-1 mIgG1 antibody+Isotype mIgG2a antibody+belzutifan;
7) Isotype mIgG1 antibody+anti-TIGIT mIgG2a antibody+belzutifan; and
8) anti-PD-1 mIgG1 antibody+anti-TIGIT mIgG2a antibody+belzutifan.

Isotype mIgG1 and anti-PD-1 antibodies were dosed intraperitoneally every 5 days at 5 mg/kg body weight. Isotype mIgG2a and anti-TIGIT antibodies were dosed intraperitoneally every 5 days at 10 mg/kg body weight. Belzutifan was dosed orally twice daily at 3 mg/kg body weight. Start of treatments was considered Day 0 and dosing based on schedules continued as described until Day 43. Caliper measurements of tumors and body weights were captured twice weekly. Statistical analyses were performed by 2-sided student t-test of final 2 groups at Day 43 when study ended.

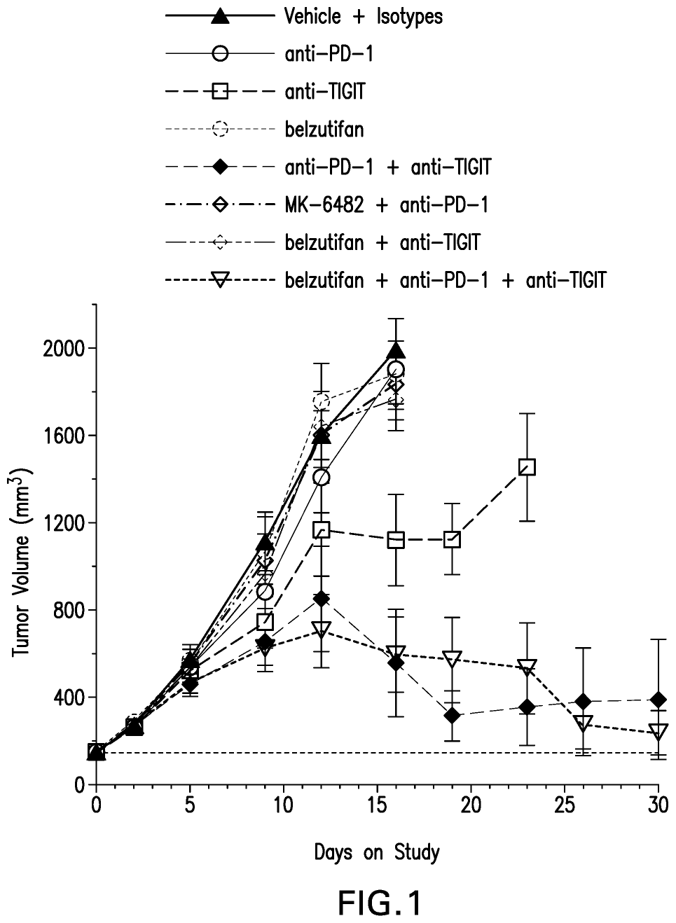

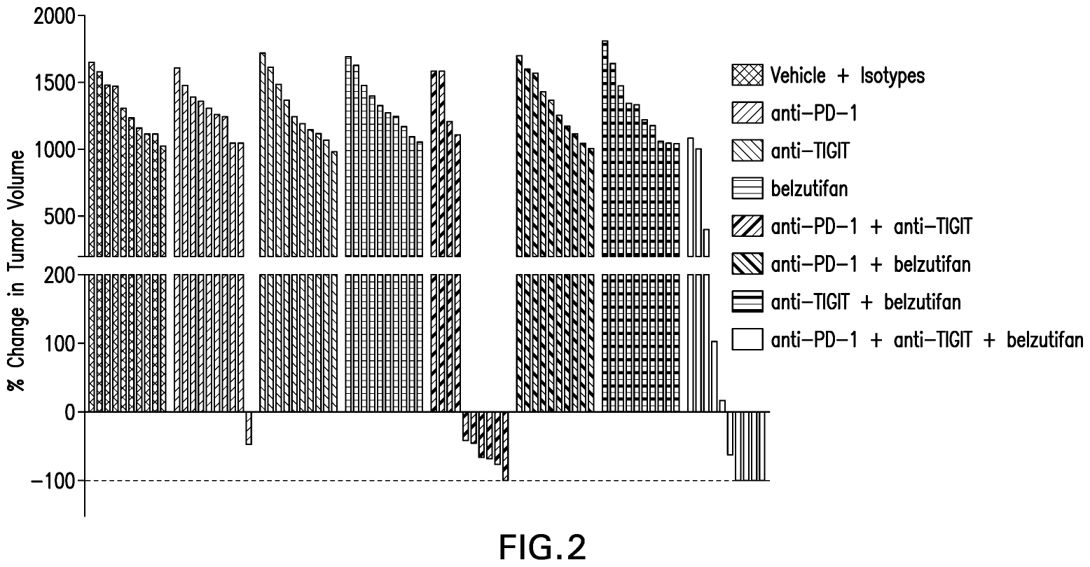

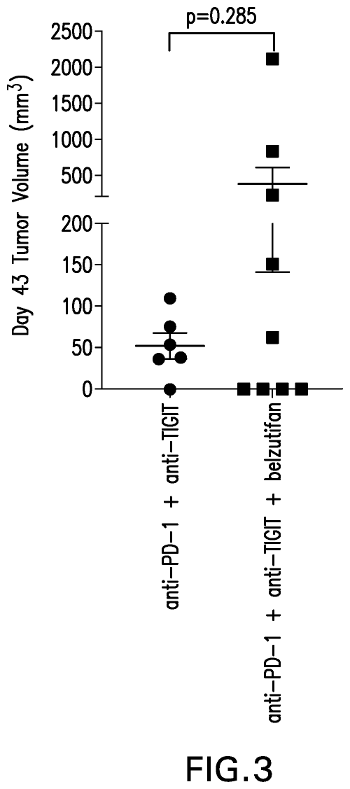

The results are shown in FIGS. 1-3 and Table 15. The combination of anti-PD-1 antibody and anti-TIGIT antibody; and combination of anti-PD-1 antibody, anti-TIGIT antibody belzutifan had similar tumor growth inhibition (TGI) at Day 16 when control Group 1 exited the study (see Table 15). The triple combination achieved 4 complete tumor regressions (CRs) and 1 partial tumor regression (PR) at the end of study while the group receiving anti-PD-1+TIGIT only achieved 1 CR and 5 PRs (see Table 15). There was no significant body weight loss or adverse events observed in any animals treated with the above therapies, indicating the treatments were well tolerated.

TABLE 15
Summary of tumor growth inhibition (TGI) % at Day 16 when
control animals exited study and the total number of partial
tumor regressions observed at end of study (Day 43).

| | | Number of complete tumor |
|---|---|---|
| | Tumor growth | regressions (CRs) and |
| | inhibition (TGI) | partial tumor regressions |
| Treatment | % at Day 16 | (PRs) at end of study |
| Anti-PD-1 | 4.8 | 0 CRs, 1 PR |
| Anti-TIGIT | 47.4 | 0 CRs, 0 PRs |
| belzutifan | 5.8 | 0 CRs, 0 PRs |
| Anti-PD-1 + anti-TIGIT | 77.9 | 1 CR, 5 PRs |
| Anti-PD-1 + belzutifan | 8.6 | 0 CRs, 0 PRs |
| anti-TIGIT + belzutifan | 12.4 | 0 CRs, 0 PRs |
| anti-PD-1 + anti-TIGIT + | 75.8 | 4 CRs, 1 PR |
| belzutifan | | |

Sequences of antibodies used in Example 2.

| | | SEQ |
|---|---|---|
| | | ID |
| | Sequences | NO. |
| Isotype | QVQLQQPGAELVRPGSSVKLSCKASGYTFN | 296 |
| mIgG1 | HYWIHWVKQGPLQGLEWIGSIDPSDSEVNY | |
| antibody- | NHKFRDKATLTVDKSSSTAYMHLSSLTSED | |
| heavy | SAVYYCAKGLRDSWGQGTTLTVSSAKTTPP | |
| chain | SVYPLAPGSAAQTNSMVTLGCLVKGYFPEP | |
| | VTVTWNSGSLSSGVHTFPAVLQSDLYTLSS | |
| | SVTVPSSTWPSETVTCNVAHPASSTKVDKK | |
| | IVPRDCGCKPCICTVPEVSSVFIFPPKPKD | |
| | VLTITLTPKVTCVVVAISKDDPEVQFSWFV | |
| | DDVEVHTAQTQPREEQFNSTFRSVSELPIM | |
| | HQDWLNGKEFKCRVNSAAFPAPIEKTISKT | |
| | KGRPKAPQVYTIPPPKEQMAKDKVSLTCMI | |
| | TDFFPEDITVEWQWNGQPAENYKNTQPIMD | |
| | TDGSYFVYSKLNVQKSNWEAGNTFTCSVLH | |
| | EGLHNHHTEKSLSHSPGK | |
| Isotype | QIVLTQSPAIMSASLGEEVTLTCSATSSVN | 297 |
| mIgG1 | FMHWYQQKSGTSPKLLIYSTSILASGVPSR | |
| antibody- | FSGSGSGTFYSLTISSVEAEDAADYYCHQW | |
| light | TSYPWTFGGGTKLEIKRADAAPTVSIFPPS | |
| chain | SEQLTSGGASVVCFLNNFYPKDINVKWKID | |
| | GSERQNGVLNSWTDQDSKDSTYSMSSTLTL | |
| | TKDEYERHNSYTCEATHKTSTSPIVKSFNR | |
| | NEC | |
| Isotype | QVQLQQPGAELVRPGSSVKLSCKASGYTFN | 298 |
| mIgG2a | HYWIHWVKQGPLQGLEWIGSIDPSDSEVNY | |
| antibody- | NHKFRDKATLTVDKSSSTAYMHLSSLTSED | |
| heavy | SAVYYCAKGLRDSWGQGTTLTVSSAKTTAP | |
| chain | SVYPLAPVCGDTTGSSVTLGCLVKGYFPEP | |
| | VTLTWNSGSLSSGVHTFPAVLQSDLYTLSS | |
| | SVTVTSSTWPSQSITCNVAHPASSTKVDKK | |
| | IEPRGPTIKPCPPCKCPAPNLLGGPSVFIF | |
| | PPKIKDVLMISLSPIVTCVVVDVSEDDPDV | |
| | QISWFVNNVEVHTAQTQTHREDYNSTLRVV | |
| | SALPIQHQDWMSGKEFKCKVNNKDLPAPIE | |
| | RTISKPKGSVRAPQVYVLPPPEEEMTKKQV | |
| | TLTCMVTDFMPEDIYVEWTNNGKTELNYKN | |
| | TEPVLDSDGSYFMYSKLRVEKKNWVERNSY | |
| | SCSVVHEGLHNHHTTKSFSRTPGK | |
| Isotype | QIVLTQSPAIMSASLGEEVTLTCSATSSVN | 299 |
| mIgG2a | FMHWYQQKSGTSPKLLIYSTSILASGVPSR | |
| antibody- | FSGSGSGTFYSLTISSVEAEDAADYYCHQW | |
| light | TSYPWTFGGGTKLEIKRADAAPTVSIFPPS | |
| chain | SEQLTSGGASVVCFLNNFYPKDINVKWKID | |
| | GSERQNGVLNSWTDQDSKDSTYSMSSTLTL | |
| | TKDEYERHNSYTCEATHKTSTSPIVKSFNR | |
| | NEC | |
| Anti-PD-1 | EVQLVESGGGLVQPGGSLKLSCAASGFTFS | 300 |
| mIgG1 | NSGLAWVRQAPEKGLEWVATITYNGTSTYY | |
| antibody- | RDSVKGRFTISRDNAKNTLYLQMSSLRSED | |
| heavy chain | TATYYCARWVPGSGNFDYWGQGTLVTVSSA | |
| | KTTPPSVYPLAPGSAAQTNSMVTLGCLVKG | |
| | YFPEPVTVTWNSGSLSSGVHTFPAVLQSDL | |
| | YTLSSSVTVPSSTWPSETVTCNVAHPASST | |
| | KVDKKIVPRDCGCKPCICTVPEVSSVFIFP | |
| | PKPKDVLTITLTPKVTCVVVAISKDDPEVQ | |
| | FSWFVDDVEVHTAQTQPREEQFNSTFRSVS | |
| | ELPIMHQDWLNGKEFKCRVNSAAFPAPIEK | |
| | TISKTKGRPKAPQVYTIPPPKEQMAKDKVS | |
| | LTCMITDFFPEDITVEWQWNGQPAENYKNT | |
| | QPIMDTDGSYFVYSKLNVQKSNWEAGNTFT | |
| | CSVLHEGLHNHHTEKSLSHSPGK | |
| Anti-PD-1 | DIVLTQSPASLAVSLGQRATISCRASQSVT | 301 |
| mIgG1 | ISRYTLMHWYQQKPGQPPKLLIYRASNLAS | |
| antibody- | GIPARFSGSGSGTDFTLNIHPVEEDDAATY | |
| light | YCQQSRESPWTFGGGTKLEIKRADAAPTVS | |
| chain | IFPPSSEQLTSGGASVVCFLNNFYPKDINV | |
| | KWKIDGSERQNGVLNSWTDQDSKDSTYSMS | |
| | STLTLTKDEYERHNSYTCEATHKTSTSPIV | |
| | KSFNRNEC | |
| Anti-TIGIT | QVQLMESGPGLVQPSQTLSLTCTVSGFPLT | 302 |
| mIgG2a | SYTVHWVRQPPGKGLEWIGAIWSSGSTDYN | |
| antibody- | SALKSRLNINRDSSKSQVFLKMNSLQTEDT | |
| heavy chain | AIYFCTKSGWAFFDYWGQGVMVTVSSAKTT | |
| | APSVYPLAPVCGDTTGSSVTLGCLVKGYFP | |
| | EPVTLTWNSGSLSSGVHTFPAVLQSDLYTL | |
| | SSSVTVTSSTWPSQSITCNVAHPASSTKVD | |
| | KKIEPRGPTIKPCPPCKCPAPNLLGGPSVF | |
| | IFPPKIKDVLMISLSPIVTCVVVDVSEDDP | |
| | DVQISWFVNNVEVHTAQTQTHREDYNSTLR | |
| | VVSALPIQHQDWMSGKEFKCKVNNKDLPAP | |
| | IERTISKPKGSVRAPQVYVLPPPEEEMTKK | |
| | QVTLTCMVTDFMPEDIYVEWTNNGKTELNY | |
| | KNTEPVLDSDGSYFMYSKLRVEKKNWVERN | |
| | SYSCSVVHEGLHNHHTTKSFSRTPGK | |
| Anti-TIGIT | DIQMTQSPSLLSASVGDRVTLNCIASQNIY | 303 |
| mIgG2a | KSLAWYQLKLGEAPKLLIYNANSLQAGIPS | |
| antibody- | RFSGSGSGTDFALTISGLQPEDVATYFCQQ | |
| light | YSGGYTFGAGTKLELKRADAAPTVSIFPPS | |
| chain | SEQLTSGGASVVCFLNNFYPKDINVKWKID | |
| | GSERQNGVLNSWTDQDSKDSTYSMSSTLTL | |
| | TKDEYERHNSYTCEATHKTSTSPIVKSFNR | |
| | NEC | |

1. A method of treating cancer, comprising administering to a human patient in need thereof a therapeutic combination comprising an effective amount of:
(a) a TIGIT antagonist;
(b) a PD-1 antagonist; and
(c) belzutifan.

2. The method of claim 1, wherein the cancer is selected from the group consisting of bladder cancer, breast cancer, colorectal cancer (CRC), esophageal cancer, gastrointestinal cancer, hepatocellular carcinoma (HCC), melanoma, non-small cell lung cancer (NSCLC), ovarian cancer, prostate cancer, and renal cell carcinoma (RCC).

3. The method of claim 2, wherein the cancer is RCC, optionally wherein the RCC is advanced RCC, optionally wherein the RCC is advanced RCC with clear cell component (ccRCC).

4-5. (canceled)

6. The method of claim 3, wherein the human patient has not received prior systemic treatment for advanced disease and/or the RCC is metastatic RCC.

7. (canceled)

8. A kit comprising:
(a) a TIGIT antagonist;
(b) a PD-1 antagonist; and
(c) belzutifan.

9-16. (canceled)

17. The method of claim 1, wherein the PD-1 antagonist and the TIGIT antagonist are co-formulated, the PD-1 antagonist and the TIGIT antagonist are in a fixed dose combination, or the PD-1 antagonist and the TIGIT antagonist are formulated separately.

18-19. (canceled)

20. The method of claim 1,
(i) wherein the PD-1 antagonist is an anti-human PD-1 monoclonal antibody or antigen binding fragment thereof, optionally wherein the anti-human PD-1 monoclonal antibody is a humanized antibody or the anti-human PD-1 monoclonal antibody is a human antibody; and/or
(ii) wherein the TIGIT antagonist is an anti-human TIGIT monoclonal antibody or antigen binding fragment thereof, optionally wherein the anti-human TIGIT monoclonal antibody is a humanized antibody, or the anti-human TIGIT monoclonal antibody is a human antibody.

21-25. (canceled)

26. The method of claim 20, wherein the anti-human PD-1 monoclonal antibody is pembrolizumab.

27-28. (canceled)

29. The method of claim 23, wherein:
(i) the anti-human TIGIT monoclonal antibody or antigen binding fragment thereof comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110;
(ii) the anti-human TIGIT monoclonal antibody or antigen binding fragment thereof comprises a heavy chain variable region comprising an amino acid sequence as set forth in SEQ ID NO: 148 and a light chain variable region comprising an amino acid sequence as set forth in SEQ ID NO: 152; or
(iii) the anti-human TIGIT monoclonal antibody or antigen binding fragment thereof comprises a light chain comprising or consisting of an amino acid sequence as set forth in SEQ ID NO: 294 and a heavy chain comprising or consisting of an amino acid sequence as set forth in SEQ ID NO: 295.

30-31. (canceled)

32. The method of claim 1, wherein:
(a) the PD-1 antagonist is pembrolizumab; and
(b) the TIGIT antagonist comprises three light chain CDRs comprising CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113 and three heavy chain CDRs comprising CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110.

33-34. (canceled)

35. The method of claim 32, wherein the TIGIT antagonist comprises a heavy chain variable region comprising an amino acid sequence as set forth in SEQ ID NO: 148 and a light chain variable region comprising an amino acid sequence as set forth in SEQ ID NO: 152, or the TIGIT antagonist comprises a light chain comprising or consisting of an amino acid sequence as set forth in SEQ ID NO: 294 and a heavy chain comprising or consisting of an amino acid sequence as set forth in SEQ ID NO: 295.

36. (canceled)

37. The method of claim 32, wherein the human patient is administered about 200 mg, about 240 mg, or about 2 mg/kg pembrolizumab, and wherein pembrolizumab is administered once every three weeks.

38-41. (canceled)

42. The method of claim 32, wherein the human patient is administered about 50, about 75, about 100, about 125, about 150, about 175, about 200, about 225, about 250, about 275, or about 300 mg of the TIGIT antagonist, and wherein the TIGIT antagonist is administered once every three weeks.

43-44. (canceled)

45. The method of claim 1, wherein the human patient is administered about 40 mg, about 80 mg, or about 120 mg belzutifan, and wherein belzutifan is administered once a day.

46. (canceled)

47. A method of treating RCC, comprising administering to a human patient in need thereof a therapeutic combination comprising:
(a) about 200 mg pembrolizumab;
(b) about 200 mg of an anti-TIGIT antibody or antigen binding fragment thereof comprising three light chain CDRs: CDRL1 having the amino acid sequence as set forth in SEQ ID NO: 111, CDRL2 having the amino acid sequence as set forth in SEQ ID NO: 112, and CDRL3 having the amino acid sequence as set forth in SEQ ID NO: 113, and three heavy chain CDRs: CDRH1 having the amino acid sequence as set forth in SEQ ID NO: 108, CDRH2 having the amino acid sequence as set forth in SEQ ID NO: 154, and CDRH3 having the amino acid sequence as set forth in SEQ ID NO: 110; and
(c) about 120 mg belzutifan.

48. The method of claim 47, wherein (a) and (b) are administered once every three weeks and (c) is administered once a day.

49. The method of claim 47,
(i) wherein (a) and (b) are administered on the same day, and wherein (a) and (b) are administered sequentially or concurrently;
(ii) wherein (a) and (b) are co-formulated; or
(iii) wherein (a) and (b) are in a fixed dose combination.

50-51. (canceled)

52. The method of claim 47, wherein the RCC is advanced RCC, optionally wherein the RCC is advanced RCC with clear cell component (ccRCC).

53. (canceled)

54. The method of claim 52, wherein the human patient has not received prior systemic treatment for advanced disease.

55. A method of treating von-Hippel Lindau disease, comprising administering to a human patient in need thereof a therapeutic combination comprising an effective amount of:
(a) a TIGIT antagonist;
(b) a PD-1 antagonist; and
(c) belzutifan.

56-91. (canceled)


SEQUENCE LISTING (303 sequences)

SEQ ID NO: 1 - protein, 15 aa, synthetic construct
RASKGVSTSGYSYLH

SEQ ID NO: 2 - protein, 7 aa, synthetic construct
LASYLES

SEQ ID NO: 3 - protein, 9 aa, synthetic construct
QHSRDLPLT

SEQ ID NO: 4 - protein, 111 aa, synthetic construct [sequence omitted]

SEQ ID NO: 5 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 5 aa, synthetic construct
NYYMY

SEQ ID NO: 7 - protein, 17 aa, synthetic construct
GINPSNGGTNFNEKFKN

SEQ ID NO: 8 - protein, 11 aa, synthetic construct
RDYRFDMGFDY

SEQ ID NO: 9 - protein, 120 aa, synthetic construct [sequence omitted]

SEQ ID NO: 10 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 11 aa, synthetic construct
RASQSVSSYLA

SEQ ID NO: 12 - protein, 7 aa, synthetic construct
DASNRAT

SEQ ID NO: 13 - protein, 9 aa, synthetic construct
QQSSNWPRT

SEQ ID NO: 14 - protein, 107 aa, synthetic construct [sequence omitted]

SEQ ID NO: 15 - protein, 214 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 5 aa, synthetic construct
NSGMH

SEQ ID NO: 17 - protein, 17 aa, synthetic construct
VIWYDGSKRYYADSVKG

SEQ ID NO: 18 - protein, 4 aa, synthetic construct
NDDY

SEQ ID NO: 19 - protein, 113 aa, synthetic construct [sequence omitted]

SEQ ID NO: 20 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 15 aa, synthetic construct
RASKSVSTSGFSYLH

SEQ ID NO: 22 - protein, 7 aa, synthetic construct
LASNLES

SEQ ID NO: 23 - protein, 9 aa, synthetic construct
QHSWELPLT

SEQ ID NO: 24 - protein, 5 aa, synthetic construct
SYYLY

SEQ ID NO: 25 - protein, 17 aa, synthetic construct
GVNPSNGGTNFSEKFKS

SEQ ID NO: 26 - protein, 11 aa, synthetic construct
RDSNYDGGFDY

SEQ ID NO: 27 - protein, 120 aa, synthetic construct [sequence omitted]

SEQ ID NO: 28 - protein, 111 aa, synthetic construct [sequence omitted]

SEQ ID NO: 29 - protein, 111 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 30 - protein, 111 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 31 - protein, 447 aa, synthetic construct; identical to SEQ ID NO: 10

SEQ ID NO: 32 - protein, 218 aa, synthetic construct; identical to SEQ ID NO: 5

SEQ ID NO: 33 - protein, 218 aa, synthetic construct
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 34 - protein, 218 aa, synthetic construct
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 35 - protein, 5 aa, synthetic construct
SDYWG

SEQ ID NO: 36 - protein, 16 aa, synthetic construct
FITYSGSTSYNPSLKS

SEQ ID NO: 37 - protein, 19 aa, synthetic construct
MPSFITLASLSTWEGYFDF

SEQ ID NO: 38 - protein, 11 aa, synthetic construct
KASQSIHKNLA

SEQ ID NO: 39 - protein, 7 aa, synthetic construct
YANSLQT

SEQ ID NO: 40 - protein, 8 aa, synthetic construct
QQYYSGWT

SEQ ID NO: 41 - protein, 127 aa, synthetic construct
EVQLQESGPGLVKPSQSLSLTCSVTGSSIASDYWGWIRKFPGNKMEWMGFITYSGSTSYNPSLKSRISITRDTSKNQFFLQLHSVTTDDTATYSCARMPSFITLASLSTWEGYFDFWGPGTMVTVSS

SEQ ID NO: 42 - protein, 106 aa, synthetic construct
DIQMTQSPSLLSASVGDRVTLNCKASQSIHKNLAWYQQKLGEAPKFLIYYANSLQTGIPSRFSGSGSGTDFTLTISGLQPEDVATYFCQQYYSGWTFGGGTKVELK

SEQ ID NO: 43 - protein, 127 aa, synthetic construct
EVQLQESGPGLVKPSETLSLTCTVSGGSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 44 - protein, 127 aa, synthetic construct
EVQLQESGPGLVKPSETLSLTCTVSGGSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 45 - protein, 127 aa, synthetic construct
EVQLQESGPGLVKPSETLSLTCTVSGGSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 46 - protein, 127 aa, synthetic construct
EVQLQESGPGLVKPSETLSLTCTVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 47 - protein, 127 aa, synthetic construct
EVQLQESGPGLVKPSETLSLTCTVSGGSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 48 - protein, 127 aa, synthetic construct
EVQLQESGPGLVKPSETLSLTCTVSGGSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 49 - protein, 127 aa, synthetic construct
EVQLQESGPGLVKPSETLSLTCTVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 50 - protein, 127 aa, synthetic construct
EVQLQESGPGLVKPSETLSLTCTVSGSSISSDYWGWIRQPPGKGLEWMGFITYSGSTSYNPSLKSRITISVDTSKNQFSLKLHSVTAADTAVYYCARMPSFITLASLSTWEGYFDFWGQGTMVTVSS

SEQ ID NO: 51 - protein, 127 aa, synthetic construct
EVQLQESGPGLVKPSETLSLTCAVSGYSISSDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 52 - protein, 127 aa, synthetic construct
EVQLQESGPGLVKPSETLSLTCAVSGYSISSDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 53 - protein, 127 aa, synthetic construct
EVQLQESGPGLVKPSETLSLTCAVSGYSISSDYWGWIRQPPGKGLEWIGFITYSGSTSYNPSLKSRITISRDTSKNQFSLKLSSVTAADTAVYYCARMPSFITLASLSTWEGYFDFWGQGTMVTVSS

SEQ ID NO: 54 - protein, 127 aa, synthetic construct
EVQLQESGPGLVKPSETLSLTCAVSGSSISSDYWGWIRQPPGKGLEWMGFITYSGSTSYNPSLKSRITISRDTSKNQFSLKLSSVTAADTAVYYCARMPSFITLASLSTWEGYFDFWGQGTMVTVSS

SEQ ID NO: 55 - protein, 127 aa, synthetic construct
EVQLQESGPGLVKPSETLSLTCAVSGYSISSDYWGWIRQPPGKGLEWIGFITYSGSTSYNPSLKSRVTISRDTSKNQFSLKLHSVTAADTAVYYCARMPSFITLASLSTWEGYFDFWGQGTMVTVSS

SEQ ID NO: 56 - protein, 127 aa, synthetic construct
EVQLQESGPGLVKPSETLSLTCAVSGYSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 57 - protein, 127 aa, synthetic construct
EVQLQESGPGLVKPSETLSLTCAVSGSSISSDYWGWIRQPPGKGLEWIGFITYSGSTSYNPSLKSRITISRDTSKNQFSLKLHSVTAADTAVYYCARMPSFITLASLSTWEGYFDFWGQGTMVTVSS

SEQ ID NO: 58 - protein, 127 aa, synthetic construct
EVQLQESGPGLVKPSETLSLTCAVSGSSISSDYWGWIRQPPGKGLEWMGFITYSGSTSYNPSLKSRITISRDTSKNQFSLKLHSVTAADTAVYYCARMPSFITLASLSTWEGYFDFWGQGTMVTVSS

SEQ ID NO: 59 - protein, 106 aa, synthetic construct
DIQMTQSPSSLSASVGDRVTITCKASQSIHKNLAWYQQKPGKAPKLLIYYANSLQTGVPSRFSGSGSGTDFTLTISSLQPEDFATYYCQQYYSGWTFGGGTKVEIK

SEQ ID NO: 60 - protein, 106 aa, synthetic construct
DIQMTQSPSSLSASVGDRVTITCKASQSIHKNLAWYQQKPGKAPKFLIYYANSLQTGVPSRFSGSGSGTDFTLTISSLQPEDFATYYCQQYYSGWTFGGGTKVEIK

SEQ ID NO: 61 - protein, 106 aa, synthetic construct
DIQMTQSPSSLSASVGDRVTITCKASQSIHKNLAWYQQKPGKAPKFLIYYANSLQTGIPSRFSGSGSGTDFTLTISSLQPEDFATYYCQQYYSGWTFGGGTKVEIK

SEQ ID NO: 62 - protein, 106 aa, synthetic construct
DIQMTQSPSSLSASVGDRVTITCKASQSIHKNLAWYQQKPGKVPKLLIYYANSLQTGVPSRFSGSGSGTDFTLTISSLQPEDVATYYCQQYYSGWTFGGGTKVEIK

SEQ ID NO: 63 - protein, 106 aa, synthetic construct
DIQMTQSPSSLSASVGDRVTITCKASQSIHKNLAWYQQKPGKVPKFLIYYANSLQTGVPSRFSGSGSGTDFTLTISSLQPEDVATYYCQQYYSGWTFGGGTKVEIK

SEQ ID NO: 64 - protein, 106 aa, synthetic construct
DIQMTQSPSSLSASVGDRVTITCKASQSIHKNLAWYQQKPGKVPKFLIYYANSLQTGIPSRFSGSGSGTDFTLTISSLQPEDVATYYCQQYYSGWTFGGGTKVEIK

SEQ ID NO: 65 - protein, 129 aa, synthetic construct
EVQLQESGPGLVKPSETLSLTCTVSGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSSAS

SEQ ID NO: 66 - protein, 127 aa, synthetic construct
EVQLQESGPGLVKPSETLSLTCTVSGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 67 - protein, 127 aa, synthetic construct
EVQLQESGPGLVKPSETLSLTCTVSGSSIASDYWGWIRKPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 68 - protein, 127 aa, synthetic construct
EVQLQESGPGLVKPSETLSLTCTVSGSSIASDYWGWIRQPPGKKLEWIGFITYSGSTSYNPSLKSRVTISRDTSKNQFSLKLSSVTAADTAVYYCARMPSFITLASLSTWEGYFDFWGQGTMVTVSS

SEQ ID NO: 69 - protein, 127 aa, synthetic construct
EVQLQESGPGLVKPSETLSLTCTVSGSSIASDYWGWIRQPPGKGMEWIGFITYSGSTSYNPSLKSRVTISRDTSKNQFSLKLSSVTAADTAVYYCARMPSFITLASLSTWEGYFDFWGQGTMVTVSS

SEQ ID NO: 70 - protein, 127 aa, synthetic construct
EVQLQESGPGLVKPSETLSLTCTVSGSSIASDYWGWIRKPPGKKMEWIGFITYSGSTSYNPSLKSRVTISRDTSKNQFSLKLSSVTAADTAVYYCARMPSFITLASLSTWEGYFDFWGQGTMVTVSS

SEQ ID NO: 71 - protein, 127 aa, synthetic construct
EVQLQESGPGLVKPSETLSLTCTVSGSSIASDYWGWIRQFPGKGLEWIGFITYSGSTSYNPSLKSRVTISRDTSKNQFSLKLSSVTADDTAVYYCARMPSFITLASLSTWEGYFDFWGQGTMVTVSS

SEQ ID NO: 72 - protein, 127 aa, synthetic construct
EVQLQESGPGLVKPSETLSLTCTVSGSSIASDYWGWIRKPPGKKMEWIGFITYSGSTSYNPSLKSRVTISVDTSKNQFSLKLSSVTAADTAVYYCARMPSFITLASLSTWEGYFDFWGQGTMVTVSS

SEQ ID NO: 73 - protein, 127 aa, synthetic construct
EVQLQESGPGLVKPSETLSLTCSVTGSSIASDYWGWIRQPPGKGLEWIGFITYSGSTSYNPSLKSRVTISRDTSKNQFSLKLSSVTAADTAVYYCARMPSFITLASLSTWEGYFDFWGQGTMVTVSS

SEQ ID NO: 74 - protein, 127 aa, synthetic construct
EVQLQQSGAGLLKPSETLSLTCSVTGSSIASDYWGWIRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 75 - protein, 127 aa, synthetic construct
EVQLQESGPGLVKPPGTLSLTCSVTGSSIASDYWGWVRQPPGKGLEWIGFITYSGSTSYNPSLKSRVTISVDTSKNQFSLKLSSVTAADTAVYYCARMPSFITLASLSTWEGYFDFWGQGTMVTVSS

SEQ ID NO: 76 - protein, 106 aa, synthetic construct; identical to SEQ ID NO: 59

SEQ ID NO: 77 - protein, 106 aa, synthetic construct; identical to SEQ ID NO: 60

SEQ ID NO: 78 - protein, 106 aa, synthetic construct
DIQMTQSPSSLSASVGDRVTITCKASQSIHKNLAWYQQKPGKAPKLLIYYANSLQTGVPSRFSGSGSGTDFTLTISSLQPEDFATYFCQQYYSGWTFGGGTKVEIK

SEQ ID NO: 79 - protein, 106 aa, synthetic construct
DIQMTQSPSSLSASVGDRVTITCKASQSIHKNLAWYQQKPGKAPKFLIYYANSLQTGVPSRFSGSGSGTDFTLTISSLQPEDFATYFCQQYYSGWTFGGGTKVEIK

SEQ ID NO: 80 - protein, 106 aa, synthetic construct
DIQMTQSPSSLSASVGDRVTITCKASQSIHKNLAWYQQKPGKAPKLLIYYANSLQTGIPSRFSGSGSGTDFTLTISSLQPEDFATYYCQQYYSGWTFGGGTKVEIK

SEQ ID NO: 81 - protein, 11 aa, synthetic construct
GYSITSDYAWN

SEQ ID NO: 82 - protein, 16 aa, synthetic construct
YISNSGSASYNPSLKS

SEQ ID NO: 83 - protein, 12 aa, synthetic construct
LIYYDYGGAMNF

SEQ ID NO: 84 - protein, 11 aa, synthetic construct
KASQGVSTTVA

SEQ ID NO: 85 - protein, 7 aa, synthetic construct
SASYRYT

SEQ ID NO: 86 - protein, 9 aa, synthetic construct
QHYYSTPWT

SEQ ID NO: 87 - protein, 121 aa, synthetic construct
DVQLQESGPGLVKPSQSLSLTCTVTGYSITSDYAWNWIRQFPGNKLEWMGYISNSGSASYNPSLKSRISITRDTSKNQFFLQLNSVTTEDTATYYCATLIYYDYGGAMNFWGQGTSVTVSS

SEQ ID NO: 88 - protein, 107 aa, synthetic construct
DIVMTQSHKFMSTSVGDRVSITCKASQGVSTTVAWYQQKPGQSPKLLIYSASYRYTGVPDRFTGSGSGTDFTFTISSVQSEDLAVYYCQHYYSTPWTFGGGTKLEIK

SEQ ID NO: 89 - protein, 7 aa, synthetic construct
YASNLQT

SEQ ID NO: 90 - protein, 7 aa, synthetic construct
YASSLQT

SEQ ID NO: 91 - protein, 7 aa, synthetic construct
YASTLQT

SEQ ID NO: 92 - protein, 7 aa, synthetic construct
YATTLQT

SEQ ID NO: 93 - protein, 7 aa, synthetic construct
YASYLQT

SEQ ID NO: 94 - protein, 7 aa, synthetic construct
YANQLQT

SEQ ID NO: 95 - protein, 7 aa, synthetic construct
YAGSLQT

SEQ ID NO: 96 - protein, 7 aa, synthetic construct
YASQLQT

SEQ ID NO: 97 - protein, 7 aa, synthetic construct
YADSLQT

SEQ ID NO: 98 - protein, 8 aa, synthetic construct
QQYYSGFT

SEQ ID NO: 99 - protein, 8 aa, synthetic construct
QQYYSGYT

SEQ ID NO: 100 - protein, 8 aa, synthetic construct
QQYYSGIT

SEQ ID NO: 101 - protein, 8 aa, synthetic construct
QQYYSGVT

SEQ ID NO: 102 - protein, 8 aa, synthetic construct
QQYYSGLT

SEQ ID NO: 103 - protein, 19 aa, synthetic construct
MPSFITLASLSTFEGYFDF

SEQ ID NO: 104 - protein, 19 aa, synthetic construct
MPSFITLASLSTYEGYFDF

SEQ ID NO: 105 - protein, 19 aa, synthetic construct
MPSFITLASLSTIEGYFDF

SEQ ID NO: 106 - protein, 19 aa, synthetic construct
MPSFITLASLSTVEGYFDF

SEQ ID NO: 107 - protein, 19 aa, synthetic construct
MPSFITLASLSTLEGYFDF

SEQ ID NO: 108 - protein, 5 aa, synthetic construct
SYVMH

SEQ ID NO: 109 - protein, 17 aa, synthetic construct
YIDPYNDGAKYNEKFKG

SEQ ID NO: 110 - protein, 10 aa, synthetic construct
GGPYGWYFDV

SEQ ID NO: 111 - protein, 11 aa, synthetic construct
RASEHIYSYLS

SEQ ID NO: 112 - protein, 7 aa, synthetic construct
NAKTLAE

SEQ ID NO: 113 - protein, 9 aa, synthetic construct
QHHFGSPLT

SEQ ID NO: 114 - protein, 119 aa, synthetic construct
EVQLQQSGPELVKPGSSVKMSCKASGYTFSSYVMHWVKQKPGQGLEWIGYIDPYNDGAKYNEKFKGKATLTSDKSSSTAYMELSSLTSEDSAVYYCARGGPYGWYFDVWGAGTTVTVSS

SEQ ID NO: 115 - protein, 107 aa, synthetic construct
DIQMTQSPASLSASVGETVTITCRASEHIYSYLSWYQQKQGKSPQLLVYNAKTLAEGVPSRFSGSGSGTQFSLKINSLQPEDFGTYYCQHHFGSPLTFGAGTTLELK

SEQ ID NO: 116 - protein, 16 aa, synthetic construct
YIDPYNRGAKYNEKFG

SEQ ID NO: 117 - protein, 16 aa, synthetic construct
YIDPYNLGAKYNEKGF

SEQ ID NO: 118 - protein, 16 aa, synthetic construct
YIDPYNKGAKYNEKFG

SEQ ID NO: 119 - protein, 16 aa, synthetic construct
YIDPYNFGAKYNEKFG

SEQ ID NO: 120 - protein, 16 aa, synthetic construct
YIDPYNSGAKYNEKFG

SEQ ID NO: 121 - protein, 16 aa, synthetic construct
YIDPYNYGAKYNEKFG

SEQ ID NO: 122 - protein, 16 aa, synthetic construct
YIDPYNVGAKYNEKFG

SEQ ID NO: 123 - protein, 17 aa, synthetic construct
YIDPYNDRAKYNEKFKG

SEQ ID NO: 124 - protein, 17 aa, synthetic construct
YIDPYNDNAKYNEKFKG

SEQ ID NO: 125 - protein, 17 aa, synthetic construct
YIDPYNDQAKYNEKFKG

SEQ ID NO: 126 - protein, 17 aa, synthetic construct
YIDPYNDEAKYNEKFKG

SEQ ID NO: 127 - protein, 17 aa, synthetic construct
YIDPYNDLAKYNEKFKG

SEQ ID NO: 128 - protein, 17 aa, synthetic construct
YIDPYNDKAKYNEKFKG

SEQ ID NO: 129 - protein, 17 aa, synthetic construct
YIDPYNDSAKYNEKFKG

SEQ ID NO: 130 - protein, 17 aa, synthetic construct
YIDPYNDYAKYNEKFKG

SEQ ID NO: 131 - protein, 17 aa, synthetic construct
YIDPYNDVAKYNEKFKG

SEQ ID NO: 132 - protein, 7 aa, synthetic construct
AAKTLAE

SEQ ID NO: 133 - protein, 7 aa, synthetic construct
YAKTLAE

SEQ ID NO: 134 - protein, 7 aa, synthetic construct
WAKTLAE

SEQ ID NO: 135 - protein, 7 aa, synthetic construct
SAKTLAE

SEQ ID NO: 136 - protein, 7 aa, synthetic construct
TAKTLAE

SEQ ID NO: 137 - protein, 7 aa, synthetic construct
IAKTLAE

SEQ ID NO: 138 - protein, 7 aa, synthetic construct
VAKTLAE

SEQ ID NO: 139 - protein, 7 aa, synthetic construct
NNKTLAE

SEQ ID NO: 140 - protein, 7 aa, synthetic construct
NIKTLAE

SEQ ID NO: 141 - protein, 7 aa, synthetic construct
NLLTLAE

SEQ ID NO: 142 - protein, 7 aa, synthetic construct
NTKTLAE

SEQ ID NO: 143 - protein, 7 aa, synthetic construct
NVKTLAE

SEQ ID NO: 144 - protein, 119 aa, synthetic construct
EVQLVQSGAEVKKPGASVKVSCKASGYTFSSYVMHWVRQAPGQRLEWIGYIDPYNDGAKYSQKFQGRVTLTRDTSASTAYMELSSLRSEDTAVYYCARGGPYGWYFDVWGQGTTVTVSS

SEQ ID NO: 145 - protein, 119 aa, synthetic construct
EVQLVQSGAEVKKPGASVKVSCKASGYTFSSYVMHWVRQAPGQRLEWIGYIDPYNDGAKYSQKFQGRVTLTSDKSASTAYMELSSLRSEDTAVYYCARGGPYGWYFDVWGQGTTVTVSS

SEQ ID NO: 146 - protein, 119 aa, synthetic construct
EVQLVQSGAEVKKPGASVKVSCKASGYTFSSYVMHWVRQAPGQGLEWIGYIDPYNDGAKYAQKFQGRVTLTRDTSTSTVYMELSSLRSEDTAVYYCARGGPYGWYFDVWGQGTTVTVSS

SEQ ID NO: 147 - protein, 119 aa, synthetic construct
EVQLVQSGAEVKKPGASVKVSCKASGYTFSSYVMHWVRQAPGQGLEWIGYIDPYNDGAKYAQKFQGRVTLTSDKSTSTVYMELSSLRSEDTAVYYCARGGPYGWYFDVWGQGTTVTVSS

SEQ ID NO: 148 - protein, 119 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSS

SEQ ID NO: 149 - protein, 119 aa, synthetic construct
EVQLVQSGAEVKKPGASVKVSCKASGYTFSSYVMHWVRQAPGQGLEWIGYIDPYNDGAKYAQKFQGRVTLTSDKSISTAYMELSRLRSDDTVVYYCARGGPYGWYFDVWGQGTTVTVSS

SEQ ID NO: 150 - protein, 107 aa, synthetic construct
DIQMTQSPSSLSASVGDRVTITCRASEHIYSYLSWYQQKPGKAPKLLIYNAKTLAEGVPSRFSGSGSGTDFTLTISSLQPEDFATYYCQHHFGSPLTFGQGTRLEIK

SEQ ID NO: 151 - protein, 107 aa, synthetic construct
DIQMTQSPSSLSASVGDRVTITCRASEHIYSYLSWYQQKPGKAPKLLIYNAKTLAEGVPSRFSGSGSGTQFTLTISSLQPEDFATYYCQHHFGSPLTFGQGTRLEIK

SEQ ID NO: 152 - protein, 107 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIK

SEQ ID NO: 153 - protein, 107 aa, synthetic construct
DIQMTQSPSSLSASVGDRVTITCRASEHIYSYLSWYQQKPGKVPKLLIYNAKTLAEGVPSRFSGSGSGTQFTLTISSLQPEDVATYYCQHHFGSPLTFGQGTRLEIK

SEQ ID NO: 154 - protein, 17 aa, synthetic construct
YIDPYNDGAKYAQKFQG

SEQ ID NO: 155 - protein, 17 aa, synthetic construct
YIDPYNDGAKYSQKFQG

SEQ ID NO: 156 - protein, 116 aa, synthetic construct
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSS

SEQ ID NO: 157 - protein, 106 aa, synthetic construct
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELK

SEQ ID NO: 158 - protein, 119 aa, synthetic construct
EVQLVESGGDLVQPGRSLKISCVASGFTFSDYYMAWVRLAPQKGLEWVASISYEGSRTHYGDSVRGRFIISRDNPKNILYLQMNSLGSEDTATYFCARHTGTLDWLVYWGQGTLVIVSS

SEQ ID NO: 159 - protein, 106 aa, synthetic construct
NIVMAQSPKSMSISAGDRVTMNCKASQNVDNNIAWYQQKPGQSPKLLIFYASNRYSGVPDRFTGGGYGTDFTLTIKSVQAEDAAFYYCQRIYNFPTFGSGTKLEIK

SEQ ID NO: 160 - protein, 19 aa, synthetic construct
  VARIANT 13: note = W, F, Y, I, V, or L
MPSFITLASLSTXEGYFDF

SEQ ID NO: 161 - protein, 7 aa, synthetic construct
  VARIANT 3: note = N, S, T, G, or D
  VARIANT 4: note = S, N, S, T, Y, or Q
YAXXLQT

SEQ ID NO: 162 - protein, 8 aa, synthetic construct
  VARIANT 7: note = W, F, Y, I, V, or L
QQYYSGXT

SEQ ID NO: 163 - protein, 127 aa, synthetic construct
  VARIANT 6: note = E or Q
  VARIANT 9: note = P or A
  VARIANT 12: note = V or L
  VARIANT 15: note = S or P
  VARIANT 16: note = Q, E, or G
  VARIANT 17: note = S or T
  VARIANT 23: note = S, T, or A
  VARIANT 25: note = T or S
  VARIANT 27: note = S, G, or Y
  VARIANT 30: note = A or S
  VARIANT 37: note = I or V
  VARIANT 39: note = K or Q
  VARIANT 40: note = F or P
  VARIANT 43: note = N or K
  VARIANT 44: note = K or G
  VARIANT 45: note = M or L
  VARIANT 48: note = M or I
  VARIANT 67: note = I or V
  VARIANT 68: note = S or T
  VARIANT 70: note = T or S
  VARIANT 71: note = R or V
  VARIANT 79: note = F or S
  VARIANT 81: note = Q or K
  VARIANT 83: note = H or S
  VARIANT 87: note = T or A
  VARIANT 88: note = D or A
  VARIANT 92: note = T or V
  VARIANT 94: note = S or Y
  VARIANT 110: note = W, F, Y, I, V or L
  VARIANT 119: note = P or Q
  VARIANT 122: note = M, V, L, A, R, N, P, Q, E, G, I, H, K, F, S, T, W or Y
  VARIANT 123: note = V, T or L
EVQLQXSGXGLXKPXXXLSLTCXVXGXSIXSDYWGWXRXXPGXXXEWXGFITYSGSTSYNPSLKSRXXIXXDTSKNQFXLXLXSVTXXDTAXYXCARMPSFITLASLSTXEGYFDFWGXGTXXTVSS

SEQ ID NO: 164 - protein, 127 aa, synthetic construct
  VARIANT 6: note = E or Q
  VARIANT 9: note = P or A
  VARIANT 12: note = V or L
  VARIANT 15: note = S or P
  VARIANT 16: note = E or G
  VARIANT 23: note = T, A, or S
  VARIANT 25: note = S or T
  VARIANT 27: note = G, S, or Y
  VARIANT 30: note = S or A
  VARIANT 37: note = I or V
  VARIANT 39: note = Q or K
  VARIANT 40: note = P or F
  VARIANT 44: note = G or K
  VARIANT 45: note = L or M
  VARIANT 48: note = I or M
  VARIANT 67: note = V or I
  VARIANT 71: note = V or R
  VARIANT 83: note = S or H
  VARIANT 88: note = A or D
  VARIANT 110: note = W, F, Y, I, V, or L
  VARIANT 122: note = M, V, L, A, R, N, P, Q, E, G, I, H, K, F, S, T, W or Y
  VARIANT 123: note = V, T or L
EVQLQXSGXGLXKPXXTLSLTCXVXGXSIXSDYWGWXRXXPGKXXEWXGFITYSGSTSYNPSLKSRXTISXDTSKNQFSLKLXSVTAXDTAVYYCARMPSFITLASLSTXEGYFDFWGQGTXXTVSS

SEQ ID NO: 165 - protein, 106 aa, synthetic construct
  VARIANT 10: note = L or S
  VARIANT 21: note = L or I
  VARIANT 22: note = N or T
  VARIANT 40: note = L or P
  VARIANT 42: note = E or K
  VARIANT 43: note = A or V
  VARIANT 46: note = F or L
  VARIANT 52: note = N, S, T, G or D
  VARIANT 53: note = S, N, T, Y or Q
  VARIANT 58: note = I or V
  VARIANT 77: note = G or S
  VARIANT 83: note = V or F
  VARIANT 87: note = F or Y
  VARIANT 95: note = W, F, Y, I, V or L
  VARIANT 105: note = L or I
DIQMTQSPSXLSASVGDRVTXXCKASQSIHKNLAWYQQKXGXXPKXLIYYAXXLQTGXPSRFSGSGSGTDFTLTISXLQPEDXATYXCQQYYSGXTFGGGTKVEXK

SEQ ID NO: 166 - protein, 106 aa, synthetic construct
  VARIANT 43: note = A or V
  VARIANT 46: note = L or F
  VARIANT 52: note = N, S, T, G or D
  VARIANT 53: note = S, N, T, Y or Q
  VARIANT 58: note = V or I
  VARIANT 83: note = F or V
  VARIANT 95: note = W, F, Y, I, V or L
DIQMTQSPSSLSASVGDRVTITCKASQSIHKNLAWYQQKPGKXPKXLIYYAXXLQTGXPSRFSGSGSGTDFTLTISSLQPEDXATYYCQQYYSGXTFGGGTKVEIK

SEQ ID NO: 167 - protein, 17 aa, synthetic construct
  VARIANT 7: note = D, R, L, K, F, S, Y or V
  VARIANT 8: note = G, R, N, Q, E, L, K, S, Y, or V
  VARIANT 12: note = N, A or S
  VARIANT 13: note = E or Q
  VARIANT 16: note = K or Q
YIDPYNXXAKYXXKFXG

SEQ ID NO: 168 - protein, 7 aa, synthetic construct
  VARIANT 1: note = N, A, V, W, S, T, R, H, G, I, or V
  VARIANT 2: note = A, N, I, L, T or V
XXKTLAE

SEQ ID NO: 169 - protein, 119 aa, synthetic construct
  VARIANT 5: note = Q or V
  VARIANT 9: note = P or A
  VARIANT 11: note = V or L
  VARIANT 12: note = V or K
  VARIANT 16: note = S or A
  VARIANT 20: note = M or V
  VARIANT 38: note = K or R
  VARIANT 40: note = K or A
  VARIANT 44: note = G or R
  VARIANT 56: note = D, R, L, K, F, S, Y or V
  VARIANT 57: note = G, R, N, Q, E, L, K, S, Y, or V
  VARIANT 61: note = N, A or S
  VARIANT 62: note = E or Q
  VARIANT 65: note = K or Q
  VARIANT 67: note = R or K
  VARIANT 68: note = A or V
  VARIANT 72: note = S or R
  VARIANT 74: note = K or T
  VARIANT 76: note = S, I , A or T
  VARIANT 79: note = A or V
  VARIANT 85: note = R or S
  VARIANT 87: note = T or R
  VARIANT 89: note = D or E
  VARIANT 91: note = S or T
  VARIANT 92: note = A or V
  VARIANT 104: note = W, A, D, E, F, G, I, K, N, Q, R, S, T, V or Y
  VARIANT 111: note = A or Q
EVQLXQSGXEXXKPGXSVKXSCKASGYTFSSYVMHWVXQXPGQXLEWIGYIDPYNXXAKYXXKFXGXXTLTXDXSXSTXYMELSXLXSXDXXVYYCARGGPYGXYFDVWGXGTTVTVSS

SEQ ID NO: 170 - protein, 119 aa, synthetic construct
  VARIANT 16: note = A or S
  VARIANT 44: note = R or G
  VARIANT 56: note = D, R, L, K, F, S, Y or V
  VARIANT 57: note = G, R, N, Q, E, L, K, S, Y, or V
  VARIANT 61: note = N, A or S
  VARIANT 62: note = E or Q
  VARIANT 65: note = K or Q
  VARIANT 72: note = R or S
  VARIANT 74: note = T or K
  VARIANT 76: note = A, T or I
  VARIANT 79: note = A or V
  VARIANT 85: note = S or R
  VARIANT 89: note = E or D
  VARIANT 92: note = A or V
  VARIANT 104: note = W, A, D, E, F, G, I, K, N, Q, R, S, T, V or Y
EVQLVQSGAEVKKPGXSVKVSCKASGYTFSSYVMHWVRQAPGQXLEWIGYIDPYNXXAKYXXKFXGRVTLTXDXSXSTXYMELSXLRSXDTXVYYCARGGPYGXYFDVWGQGTTVTVSS

SEQ ID NO: 171 - protein, 107 aa, synthetic construct
  VARIANT 9: note = A or S
  VARIANT 17: note = E or D
  VARIANT 18: note = T or R
  VARIANT 40: note = Q or P
  VARIANT 43: note = S, A or V
  VARIANT 45: note = Q or K
  VARIANT 48: note = V or I
  VARIANT 50: note = N, A, Y, W, S, T, I or V
  VARIANT 51: note = A, N, I, L, T or V
  VARIANT 70: note = Q or D
  VARIANT 72: note = S or T
  VARIANT 74: note = K or T
  VARIANT 76: note = N or S
  VARIANT 83: note = F or V
  VARIANT 84: note = G or A
  VARIANT 100: note = A or Q
  VARIANT 103: note = T or R
  VARIANT 106: note = L or I
DIQMTQSPXSLSASVGXXVTITCRASEHIYSYLSWYQQKXGKXPXLLXYXXKTLAEGVPSRFSGSGSGTXFXLXIXSLQPEDXXTYYCQHHFGSPLTFGXGTXLEXK

SEQ ID NO: 172 - protein, 107 aa, synthetic construct
  VARIANT 43: note = A or V
  VARIANT 50: note = N, A, Y, W, S, T, I or V
  VARIANT 51: note = A, N, I, L, T or V
  VARIANT 70: note = D or Q
  VARIANT 83: note = F or V
DIQMTQSPSSLSASVGDRVTITCRASEHIYSYLSWYQQKPGKXPKLLIYXXKTLAEGVPSRFSGSGSGTXFTLTISSLQPEDXATYYCQHHFGSPLTFGQGTRLEIK

SEQ ID NO: 173 - protein, 10 aa, synthetic construct
  VARIANT 6: note = W, A, D, E, F, G, I, K, N, Q, R, S, T, V or Y
GGPYGXYFDV

SEQ ID NO: 174 - protein, 10 aa, synthetic construct
GGPYGAYFDV

SEQ ID NO: 175 - protein, 10 aa, synthetic construct
GGPYGDYFDV

SEQ ID NO: 176 - protein, 10 aa, synthetic construct
GGPYGEYFDV

SEQ ID NO: 177 - protein, 10 aa, synthetic construct
GGPYGFYFDV

SEQ ID NO: 178 - protein, 10 aa, synthetic construct
GGPYGGYFDV

SEQ ID NO: 179 - protein, 10 aa, synthetic construct
GGPYGIYFDV

SEQ ID NO: 180 - protein, 10 aa, synthetic construct
GGPYGKYFDV

SEQ ID NO: 181 - protein, 10 aa, synthetic construct
GGPYGNYFDV

SEQ ID NO: 182 - protein, 10 aa, synthetic construct
GGPYGQYFDV

SEQ ID NO: 183 - protein, 10 aa, synthetic construct
GGPYGRYFDV

SEQ ID NO: 184 - protein, 10 aa, synthetic construct
GGPYGSYFDV

SEQ ID NO: 185 - protein, 10 aa, synthetic construct
GGPYGTYFDV

SEQ ID NO: 186 - protein, 10 aa, synthetic construct
GGPYGVYFDV

SEQ ID NO: 187 - protein, 10 aa, synthetic construct
GGPYGYYFDV

SEQ ID NO: 188 - protein, 5 aa, synthetic construct
GAWMD

SEQ ID NO: 189 - protein, 19 aa, synthetic construct
EIRTKVNNHATNYGESVKG

SEQ ID NO: 190 - protein, 10 aa, synthetic construct
ALYDGFYFDY

SEQ ID NO: 191 - protein, 12 aa, synthetic construct
SASSSVSSGYLY

SEQ ID NO: 192 - protein, 7 aa, synthetic construct
GTSTLAS

SEQ ID NO: 193 - protein, 9 aa, synthetic construct
HQWSSFPYT

SEQ ID NO: 194 - protein, 121 aa, synthetic construct
EVKLEESGGGLVQPGGSMKLSCVASGFTFSGAWMDWVRQSPEKGLEWVAEIRTKVNNHATNYGESVKGRFTISRDDSKSSVYLQMNNLRAEDSGIYYCRGALYDGFYFDYWGQGTTLTVSS

SEQ ID NO: 195 - protein, 108 aa, synthetic construct
QIVLTQSPAIMSASPGEKVNLTCSASSSVSSGYLYWYQQKPGSSPKLWIYGTSTLASGVPARFSGSGSGTSYSLTISNMEAEDAASYFCHQWSSFPYTFGGGTKLEMK

SEQ ID NO: 196 - protein, 121 aa, synthetic construct
  VARIANT 33: note = W, A, R, N, D, Q, E, G, H, I, L, K, F, P, S, T, Y, or V
  VARIANT 34: note = M, V, L, I, G, A, S, or T
  VARIANT 76: note = D, A, R, N, Q, E, G, H, I, L, K, F, S, T, Y, or V
  VARIANT 79: note = S or N
  VARIANT 80: note = T or S
  VARIANT 85: note = M or L
  VARIANT 86: note = N or S
  VARIANT 87: note = S or N
  VARIANT 93: note = T or S
  VARIANT 104: note = D, A, R, N, Q, E, G, H, I, L, K, F, P, S, T, W, Y, or V
  VARIANT 105: note = G, A, R, N, D, Q, E, H, I, L, K, F, P, S, T, W, Y, or V
EVQLVESGGGLVQPGGSLKLSCAASGFTFSGAXXDWVRQAPGKGLEWVAEIRTKVNNHATNYGESVKGRFTISRDXSKXXVYLQXXXLRAEDXAVYYCRGALYXXFYFDYWGQGTLVTVSS

SEQ ID NO: 197 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDGFYFDYWGQGTLVTVSS

SEQ ID NO: 198 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDGFYFDYWGQGTLVTVSS

SEQ ID NO: 199 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDGFYFDYWGQGTLVTVSS

SEQ ID NO: 200 - protein, 108 aa, synthetic construct
  VARIANT 20: note = T or S
  VARIANT 46: note = K or R
  VARIANT 48: note = W, A, R, N, D, Q, E, G, H, I, L, K, F, P, S, T, Y, or V
  VARIANT 59: note = V or I
  VARIANT 79: note = L, V, or I
  VARIANT 84: note = F, V, L, I, or T
  VARIANT 92: note = W, A, R, N, D, Q, E, G, H, I, L, K, F, P, S, T, Y, or V
  VARIANT 107: note = I or L
EIVLTQSPATLSLSPGERAXLSCSASSSVSSGYLYWYQQKPGQAPXLXIYGTSTLASGXPARFSGSGSGTDYTLTISSXEPEDXAVYYCHQXSSFPYTFGQGTKLEXK

SEQ ID NO: 201 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQWSSFPYTFGQGTKLEIK

SEQ ID NO: 202 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQWSSFPYTFGQGTKLEIK

SEQ ID NO: 203 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQWSSFPYTFGQGTKLEIK

SEQ ID NO: 204 - protein, 5 aa, synthetic construct
EFTMH

SEQ ID NO: 205 - protein, 17 aa, synthetic construct
GLKPDNGGISYNQKFKG

SEQ ID NO: 206 - protein, 10 aa, synthetic construct
GAYYRYDADY

SEQ ID NO: 207 - protein, 11 aa, synthetic construct
KASQDVKTAVA

SEQ ID NO: 208 - protein, 7 aa, synthetic construct
SASYRNT

SEQ ID NO: 209 - protein, 9 aa, synthetic construct
QQHYSTPFT

SEQ ID NO: 210 - protein, 119 aa, synthetic construct
EVQLQQSGPELVKPGASVKISCKTSGYTFTEFTMHWVKQSHGKSLEWIGGLKPDNGGISYNQKFKGRATLAVDKSSNTAYMELRSLTSEDSAVYYCARGAYYRYDADYWGQGTTLTVSS

SEQ ID NO: 211 - protein, 107 aa, synthetic construct
DIVLTQSHKFMSTSVGDRVSITCKASQDVKTAVAWYQQKSGQSPKLLIYSASYRNTGVPDRFTGSGSGTDFTFTIDSVQAEDLAVYFCQQHYSTPFTFGTGTKLELK

SEQ ID NO: 212 - protein, 119 aa, synthetic construct
  VARIANT 24: note = T or V
  VARIANT 34: note = M, V, L, I, G, A, S, or T
  VARIANT 38: note = K or R
  VARIANT 55: note = N, A, R, D, Q, E, G, H, I, L, K, F, P, S, T, W, Y, or V
  VARIANT 56: note = G, A, R, N, D, Q, E, H, I, L, K, F, P, S, T, W, Y, or V
  VARIANT 74: note = K, T, D, or S
  VARIANT 77: note = N or S
  VARIANT 81: note = M, V, L, I, G, A, S, or T
  VARIANT 91: note = T or S
  VARIANT 105: note = D, A, R, N, Q, E, G, H, I, L, K, F, P, S, T, W, Y, or V
  VARIANT 106: note = A, R, N, D, Q, E, G, H, I, L, K, F, P, S, T, W, Y, V, or M
EVQLVQSGAEVKKPGASVKISCKXSGYTFTEFTXHWVXQAPGKGLEWIGGLKPDXXGISYNQKFKGRATLTVDXSTXTAYXELSSLRSEDXAVYYCARGAYYRYXXDYWGQGTLVTVSS

SEQ ID NO: 213 - protein, 119 aa, synthetic construct
EVQLVQSGAEVKKPGASVKISCKTSGYTFTEFTMHWVKQAPGKGLEWIGGLKPDNGGISYNQKFKGRATLTVDKSTNTAYMELSSLRSEDTAVYYCARGAYYRYDADYWGQGTLVTVSS

SEQ ID NO: 214 - protein, 119 aa, synthetic construct
EVQLVQSGAEVKKPGASVKISCKTSGYTFTEFTMHWVRQAPGKGLEWIGGLKPDNGGISYNQKFKGRATLTVDKSTSTAYMELSSLRSEDTAVYYCARGAYYRYDADYWGQGTLVTVSS

SEQ ID NO: 215 - protein, 119 aa, synthetic construct
EVQLVQSGAEVKKPGASVKISCKVSGYTFTEFTMHWVRQAPGKGLEWIGGLKPDNGGISYNQKFKGRATLTVDTSTSTAYMELSSLRSEDTAVYYCARGAYYRYDADYWGQGTLVTVSS

SEQ ID NO: 216 - protein, 107 aa, synthetic construct
  VARIANT 55: note = N, Q, D, or E
  VARIANT 56: note = T, S, or A
  VARIANT 60: note = D or S
  VARIANT 73: note = F or L
DIQLTQSPSSLSASVGDRVTITCKASQDVKTAVAWYQQKPGKAPKLLIYSASYRXXGVPXRFSGSGSGTDFTXTISSLQPEDFATYYCQQHYSTPFTFGQGTKLEIK

SEQ ID NO: 217 - protein, 107 aa, synthetic construct
DIQLTQSPSSLSASVGDRVTITCKASQDVKTAVAWYQQKPGKAPKLLIYSASYRNTGVPDRFSGSGSGTDFTFTISSLQPEDFATYYCQQHYSTPFTFGQGTKLEIK

SEQ ID NO: 218 - protein, 107 aa, synthetic construct
DIQLTQSPSSLSASVGDRVTITCKASQDVKTAVAWYQQKPGKAPKLLIYSASYRNTGVPSRFSGSGSGTDFTFTISSLQPEDFATYYCQQHYSTPFTFGQGTKLEIK

SEQ ID NO: 219 - protein, 107 aa, synthetic construct
DIQLTQSPSSLSASVGDRVTITCKASQDVKTAVAWYQQKPGKAPKLLIYSASYRNTGVPSRFSGSGSGTDFTLTISSLQPEDFATYYCQQHYSTPFTFGQGTKLEIK

SEQ ID NO: 220 - protein, 10 aa, synthetic construct
ALYEGFYFDY

SEQ ID NO: 221 - protein, 10 aa, synthetic construct
ALYDAFYFDY

SEQ ID NO: 222 - protein, 10 aa, synthetic construct
ALYDSFYFDY

SEQ ID NO: 223 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYEGFYFDYWGQGTLVTVSS

SEQ ID NO: 224 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDAFYFDYWGQGTLVTVSS

SEQ ID NO: 225 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSTVYLQMNSLRAEDTAVYYCRGALYDSFYFDYWGQGTLVTVSS

SEQ ID NO: 226 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDEFYFDYWGQGTLVTVSS

SEQ ID NO: 227 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDAFYFDYWGQGTLVTVSS

SEQ ID NO: 228 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKSSVYLQMNSLRAEDTAVYYCRGALYDSFYFDYWGQGTLVTVSS

SEQ ID NO: 229 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYEGFYFDYWGQGTLVTVSS

SEQ ID NO: 230 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDAFYFDYWGQGTLVTVSS

SEQ ID NO: 231 - protein, 121 aa, synthetic construct
EVQLVESGGGLVQPGGSLKLSCAASGFTFSGAWMDWVRQAPGKGLEWVAEIRTKVNNHATNYGESVKGRFTISRDDSKNTVYLQMNSLRAEDTAVYYCRGALYDSFYFDYWGQGTLVTVSS

SEQ ID NO: 232 - protein, 9 aa, synthetic construct
HQASSFPYT

SEQ ID NO: 233 - protein, 9 aa, synthetic construct
HQDSSFPYT

SEQ ID NO: 234 - protein, 9 aa, synthetic construct
HQESSFPYT

SEQ ID NO: 235 - protein, 9 aa, synthetic construct
HQFSSFPYT

SEQ ID NO: 236 - protein, 9 aa, synthetic construct
HQGSSFPYT

SEQ ID NO: 237 - protein, 9 aa, synthetic construct
HQHSSFPYT

SEQ ID NO: 238 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQASSFPYTFGQGTKLEIK

SEQ ID NO: 239 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQDSSFPYTFGQGTKLEIK

SEQ ID NO: 240 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQESSFPYTFGQGTKLEIK

SEQ ID NO: 241 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQFSSFPYTFGQGTKLEIK

SEQ ID NO: 242 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQGSSFPYTFGQGTKLEIK

SEQ ID NO: 243 - protein, 108 aa, synthetic construct
EIVLTQSPATLSLSPGERATLSCSASSSVSSGYLYWYQQKPGQAPKLWIYGTSTLASGVPARFSGSGSGTDYTLTISSLEPEDFAVYYCHQHSSFPYTFGQGTKLEIK

SEQ ID NO: 244 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQASSFPYTFGQGTKLEIK

SEQ ID NO: 245 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQDSSFPYTFGQGTKLEIK

SEQ ID NO: 246 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQESSFPYTFGQGTKLEIK

SEQ ID NO: 247 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQFSSFPYTFGQGTKLEIK

SEQ ID NO: 248 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQGSSFPYTFGQGTKLEIK

SEQ ID NO: 249 - protein, 108 aa, synthetic construct
EIVLTQSPATLSLSPGERATLSCSASSSVSSGYLYWYQQKPGQAPRLWIYGTSTLASGVPARFSGSGSGTDYTLTISSLEPEDFAVYYCHQHSSFPYTFGQGTKLEIK

SEQ ID NO: 250 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQASSFPYTFGQGTKLEIK

SEQ ID NO: 251 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQDSSFPYTFGQGTKLEIK

SEQ ID NO: 252 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQESSFPYTFGQGTKLEIK

SEQ ID NO: 253 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQFSSFPYTFGQGTKLEIK

SEQ ID NO: 254 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQGSSFPYTFGQGTKLEIK

SEQ ID NO: 255 - protein, 108 aa, synthetic construct
EIVLTQSPATLSLSPGERATLSCSASSSVSSGYLYWYQQKPGQAPRLWIYGTSTLASGIPARFSGSGSGTDYTLTISSLEPEDFAVYYCHQHSSFPYTFGQGTKLEIK

SEQ ID NO: 256 - protein, 17 aa, synthetic construct
GLKPDQGGISYNQKFKG

SEQ ID NO: 257 - protein, 17 aa, synthetic construct
GLKPDDGGISYNQKFKG

SEQ ID NO: 258 - protein, 17 aa, synthetic construct
GLKPDNAGISYNQKFKG

SEQ ID NO: 259 - protein, 17 aa, synthetic construct
GLKPDTGGISYNQKFKG

SEQ ID NO: 260 - protein, 17 aa, synthetic construct
GLKPDSGGISYNQKFKG

SEQ ID NO: 261 - protein, 17 aa, synthetic construct
GLKPDGGGISYNQKFKG

SEQ ID NO: 262 - protein, 17 aa, synthetic construct
GLKPDNSGISYNQKFKG

SEQ ID NO: 263 - protein, 17 aa, synthetic construct
GLKPDNTGISYNQKFKG

SEQ ID NO: 264 - protein, 119 aa, synthetic construct
EVQLVQSGAEVKKPGASVKISCKTSGYTFTEFTMHWVKQAPGKGLEWIGGLKPDQGGISYNQKFKGRATLTVDKSTNTAYMELSSLRSEDTAVYYCARGAYYRYDADYWGQGTLVTVSS

SEQ ID NO: 265 - protein, 119 aa, synthetic construct
EVQLVQSGAEVKKPGASVKISCKTSGYTFTEFTMHWVKQAPGKGLEWIGGLKPDDGGISYNQKFKGRATLTVDKSTNTAYMELSSLRSEDTAVYYCARGAYYRYDADYWGQGTLVTVSS

SEQ ID NO: 266 - protein, 119 aa, synthetic construct
EVQLVQSGAEVKKPGASVKISCKTSGYTFTEFTMHWVKQAPGKGLEWIGGLKPDNAGISYNQKFKGRATLTVDKSTNTAYMELSSLRSEDTAVYYCARGAYYRYDADYWGQGTLVTVSS

SEQ ID NO: 267 - protein, 119 aa, synthetic construct
EVQLVQSGAEVKKPGASVKISCKTSGYTFTEFTMHWVKQAPGKGLEWIGGLKPDTGGISYNQKFKGRATLTVDKSTNTAYMELSSLRSEDTAVYYCARGAYYRYDADYWGQGTLVTVSS

SEQ ID NO: 268 - protein, 119 aa, synthetic construct
EVQLVQSGAEVKKPGASVKISCKTSGYTFTEFTMHWVKQAPGKGLEWIGGLKPDSGGISYNQKFKGRATLTVDKSTNTAYMELSSLRSEDTAVYYCARGAYYRYDADYWGQGTLVTVSS

SEQ ID NO: 269 - protein, 119 aa, synthetic construct
EVQLVQSGAEVKKPGASVKISCKTSGYTFTEFTMHWVKQAPGKGLEWIGGLKPDGGGISYNQKFKGRATLTVDKSTNTAYMELSSLRSEDTAVYYCARGAYYRYDADYWGQGTLVTVSS

SEQ ID NO: 270 - protein, 119 aa, synthetic construct
EVQLVQSGAEVKKPGASVKISCKTSGYTFTEFTMHWVKQAPGKGLEWIGGLKPDNSGISYNQKFKGRATLTVDKSTNTAYMELSSLRSEDTAVYYCARGAYYRYDADYWGQGTLVTVSS

SEQ ID NO: 271 - protein, 119 aa, synthetic construct
EVQLVQSGAEVKKPGASVKISCKTSGYTFTEFTMHWVKQAPGKGLEWIGGLKPDNTGISYNQKFKGRATLTVDKSTNTAYMELSSLRSEDTAVYYCARGAYYRYDADYWGQGTLVTVSS

SEQ ID NO: 272 - protein, 119 aa, synthetic construct
EVQLVQSGAEVKKPGASVKISCKTSGYTFTEFTMHWVRQAPGKGLEWIGGLKPDQGGISYNQKFKGRATLTVDKSTSTAYMELSSLRSEDTAVYYCARGAYYRYDADYWGQGTLVTVSS

SEQ ID NO: 273 - protein, 119 aa, synthetic construct
EVQLVQSGAEVKKPGASVKISCKTSGYTFTEFTMHWVRQAPGKGLEWIGGLKPDDGGISYNQKFKGRATLTVDKSTSTAYMELSSLRSEDTAVYYCARGAYYRYDADYWGQGTLVTVSS

SEQ ID NO: 274 - protein, 119 aa, synthetic construct
EVQLVQSGAEVKKPGASVKISCKTSGYTFTEFTMHWVRQAPGKGLEWIGGLKPDNAGISYNQKFKGRATLTVDKSTSTAYMELSSLRSEDTAVYYCARGAYYRYDADYWGQGTLVTVSS

SEQ ID NO: 275 - protein, 119 aa, synthetic construct
EVQLVQSGAEVKKPGASVKISCKTSGYTFTEFTMHWVRQAPGKGLEWIGGLKPDTGGISYNQKFKGRATLTVDKSTSTAYMELSSLRSEDTAVYYCARGAYYRYDADYWGQGTLVTVSS

SEQ ID NO: 276 - protein, 119 aa, synthetic construct
EVQLVQSGAEVKKPGASVKISCKTSGYTFTEFTMHWVRQAPGKGLEWIGGLKPDSGGISYNQKFKGRATLTVDKSTSTAYMELSSLRSEDTAVYYCARGAYYRYDADYWGQGTLVTVSS

SEQ ID NO: 277 - protein, 119 aa, synthetic construct
EVQLVQSGAEVKKPGASVKISCKTSGYTFTEFTMHWVRQAPGKGLEWIGGLKPDGGGISYNQKFKGRATLTVDKSTSTAYMELSSLRSEDTAVYYCARGAYYRYDADYWGQGTLVTVSS

SEQ ID NO: 278 - protein, 119 aa, synthetic construct
EVQLVQSGAEVKKPGASVKISCKTSGYTFTEFTMHWVRQAPGKGLEWIGGLKPDNSGISYNQKFKGRATLTVDKSTSTAYMELSSLRSEDTAVYYCARGAYYRYDADYWGQGTLVTVSS

SEQ ID NO: 279 - protein, 119 aa, synthetic construct
EVQLVQSGAEVKKPGASVKISCKTSGYTFTEFTMHWVRQAPGKGLEWIGGLKPDNTGISYNQKFKGRATLTVDKSTSTAYMELSSLRSEDTAVYYCARGAYYRYDADYWGQGTLVTVSS

SEQ ID NO: 280 - protein, 119 aa, synthetic construct
EVQLVQSGAEVKKPGASVKISCKVSGYTFTEFTMHWVRQAPGKGLEWIGGLKPDQGGISYNQKFKGRATLTVDTSTSTAYMELSSLRSEDTAVYYCARGAYYRYDADYWGQGTLVTVSS

SEQ ID NO: 281 - protein, 119 aa, synthetic construct
EVQLVQSGAEVKKPGASVKISCKVSGYTFTEFTMHWVRQAPGKGLEWIGGLKPDDGGISYNQKFKGRATLTVDTSTSTAYMELSSLRSEDTAVYYCARGAYYRYDADYWGQGTLVTVSS

SEQ ID NO: 282 - protein, 119 aa, synthetic construct
EVQLVQSGAEVKKPGASVKISCKVSGYTFTEFTMHWVRQAPGKGLEWIGGLKPDNAGISYNQKFKGRATLTVDTSTSTAYMELSSLRSEDTAVYYCARGAYYRYDADYWGQGTLVTVSS

SEQ ID NO: 283 - protein, 119 aa, synthetic construct
EVQLVQSGAEVKKPGASVKISCKVSGYTFTEFTMHWVRQAPGKGLEWIGGLKPDTGGISYNQKFKGRATLTVDTSTSTAYMELSSLRSEDTAVYYCARGAYYRYDADYWGQGTLVTVSS

SEQ ID NO: 284 - protein, 119 aa, synthetic construct
EVQLVQSGAEVKKPGASVKISCKVSGYTFTEFTMHWVRQAPGKGLEWIGGLKPDSGGISYNQKFKGRATLTVDTSTSTAYMELSSLRSEDTAVYYCARGAYYRYDADYWGQGTLVTVSS

SEQ ID NO: 285 - protein, 119 aa, synthetic construct
EVQLVQSGAEVKKPGASVKISCKVSGYTFTEFTMHWVRQAPGKGLEWIGGLKPDGGGISYNQKFKGRATLTVDTSTSTAYMELSSLRSEDTAVYYCARGAYYRYDADYWGQGTLVTVSS

SEQ ID NO: 286 - protein, 119 aa, synthetic construct
EVQLVQSGAEVKKPGASVKISCKVSGYTFTEFTMHWVRQAPGKGLEWIGGLKPDNSGISYNQKFKGRATLTVDTSTSTAYMELSSLRSEDTAVYYCARGAYYRYDADYWGQGTLVTVSS

SEQ ID NO: 287 - protein, 119 aa, synthetic construct
EVQLVQSGAEVKKPGASVKISCKVSGYTFTEFTMHWVRQAPGKGLEWIGGLKPDNTGISYNQKFKGRATLTVDTSTSTAYMELSSLRSEDTAVYYCARGAYYRYDADYWGQGTLVTVSS

SEQ ID NO: 288 - protein, 15 aa, synthetic construct
SSTTAQVNWEQQDQL

SEQ ID NO: 289 - protein, 9 aa, synthetic construct
IYHTYPDGT

SEQ ID NO: 290 - protein, 5 aa, synthetic construct
GRIFL

SEQ ID NO: 291 - protein, 330 aa, synthetic construct [sequence omitted]

SEQ ID NO: 292 - protein, 325 aa, synthetic construct
TKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 293 - protein, 105 aa, synthetic construct [sequence omitted]

SEQ ID NO: 294 - protein, 214 aa, synthetic construct
DIQMTQSPSSLSASVGDRVTITCRASEHIYSYLSWYQQKPGKVPKLLIYNAKTLAEGVPSRFSGSGSGTDFTLTISSLQPEDVATYYCQHHFGSPLTFGQGTRLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 295 - protein, 449 aa, synthetic construct
EVQLVQSGAEVKKPGSSVKVSCKASGYTFSSYVMHWVRQAPGQGLEWIGYIDPYNDGAKYAQKFQGRVTLTSDKSTSTAYMELSSLRSEDTAVYYCARGGPYGWYFDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 296 - protein, 438 aa, synthetic construct
QVQLQQPGAELVRPGSSVKLSCKASGYTFNHYWIHWVKQGPLQGLEWIGSIDPSDSEVNYNHKFRDKATLTVDKSSSTAYMHLSSLTSEDSAVYYCAKGLRDSWGQGTTLTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 297 - protein, 213 aa, synthetic construct
QIVLTQSPAIMSASLGEEVTLTCSATSSVNFMHWYQQKSGTSPKLLIYSTSILASGVPSRFSGSGSGTFYSLTISSVEAEDAADYYCHQWTSYPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 298 - protein, 444 aa, synthetic construct
QVQLQQPGAELVRPGSSVKLSCKASGYTFNHYWIHWVKQGPLQGLEWIGSIDPSDSEVNYNHKFRDKATLTVDKSSSTAYMHLSSLTSEDSAVYYCAKGLRDSWGQGTTLTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 299 - protein, 213 aa, synthetic construct; identical to SEQ ID NO: 297

SEQ ID NO: 300 - protein, 443 aa, synthetic construct
EVQLVESGGGLVQPGGSLKLSCAASGFTFSNSGLAWVRQAPEKGLEWVATITYNGTSTYYRDSVKGRFTISRDNAKNTLYLQMSSLRSEDTATYYCARWVPGSGNFDYWGQGTLVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVAISKDDPEVQFSWFVDDVEVHTAQTQPREEQFNSTFRSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 301 - protein, 218 aa, synthetic construct
DIVLTQSPASLAVSLGQRATISCRASQSVTISRYTLMHWYQQKPGQPPKLLIYRASNLASGIPARFSGSGSGTDFTLNIHPVEEDDAATYYCQQSRESPWTFGGGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 302 - protein, 446 aa, synthetic construct
QVQLMESGPGLVQPSQTLSLTCTVSGFPLTSYTVHWVRQPPGKGLEWIGAIWSSGSTDYNSALKSRLNINRDSSKSQVFLKMNSLQTEDTAIYFCTKSGWAFFDYWGQGVMVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 303 - protein, 213 aa, synthetic construct
DIQMTQSPSLLSASVGDRVTLNCIASQNIYKSLAWYQLKLGEAPKLLIYNANSLQAGIPSRFSGSGSGTDFALTISGLQPEDVATYFCQQYSGGYTFGAGTKLELKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC